(19) United States
(12) Patent Application Publication
(10) Pub. No.: US 2023/0303305 A1
(43) Pub. Date: Sep. 28, 2023

(54) METHOD AND PACKAGE FOR REDUCING THE DEGRADATION OF A DRUG AND/OR EXCIPIENT, E.G. POLYSORBATE STABILIZER, IN A PHARMACEUTICAL PRODUCT

(71) Applicant: SiO2 Medical Products, Inc., Auburn, AL (US)

(72) Inventors: Robert S. Abrams, Albany, NY (US); Christopher Weikart, Auburn, AL (US)

(21) Appl. No.: 18/018,868

(22) PCT Filed: Jul. 28, 2021
(86) PCT No.: PCT/US2021/043516

§ 371 Date: Jan. 30, 2023

Related U.S. Application Data
(60) Provisional application No. 63/057,595, filed on Jul. 28, 2020.
(60) Provisional application No. 63/063,207, filed on Aug. 7, 2020.
(60) Provisional application No. 63/087,172, filed on Oct. 2, 2020.
(60) Provisional application No. 63/165,664, filed on Mar. 24, 2021.

(51) Int. Cl. B65D 81/24; A61M 5/315; C09D 5/00; C09D 183/04; C09D 183/16; C08J 7/06; A61J 1/14; B65B 3/00; C23C 16/26; C23C 16/50; C23C 16/32; C23C 16/34; C23C 16/04; C03C 17/00; C03C 17/30; C03C 17/22; C03C 17/245; C03C 17/34

(52) U.S. Cl. CPC B65D 81/24; A61M 5/31513; C09D 5/00; C09D 183/04; C09D 183/16; C08J 7/065; A61J 1/1468; B65B 3/003; C23C 16/26; C23C 16/50; C23C 16/325; C23C 16/345; C23C 16/045; C03C 17/004; C03C 17/30; C03C 17/225; C03C 17/245; C03C 17/22; C03C 17/3435; C03C 17/3441; C08J 2345/00; C08J 2353/00; C03C 2218/153; C03C 2217/70; C03C 2217/281; C03C 2217/282

(57) ABSTRACT

The present disclosure is directed to a vessel having a lumen defined at least in part by a wall, the wall having an interior surface facing the lumen, and the interior surface comprising a coating. The coating is configured to prevent the leaching of metal ions from the vessel wall into a fluid contained within the lumen and stored in contact with the coating. The vessels are used to store an aqueous drug product, typically an aqueous drug product containing an active agent and one or more excepients, such as a polysorbate stabilizer. The coated vessels are effective to reduce degradation of the active agent and/or one or more excipients, e.g. a polysorbate stabilizer.

The present application claims priority to U.S. Provisional Patent Application No. 63/057,595, filed on Jul. 28, 2020; U.S. Provisional Patent Application No. 63/063,207, filed on Aug. 7, 2020; U.S. Provisional Patent Application No. 63/087,172, filed on Oct. 2, 2020; and U.S. Provisional Patent Application No. 63/165,664, filed on Mar. 24, 2021; the entireties of which are incorporated herein by reference.

### FIELD OF THE INVENTION

The present invention relates to the technical field of coated surfaces, for example interior surfaces of pharmaceutical packages or other vessels for storing or other contact with fluids. Examples of suitable fluids include foods, nutritional supplements, drugs, inhalation anaesthetics, diagnostic test materials, biologically active compounds or body fluids, for example blood. The present invention also relates to a pharmaceutical package or other vessel, and to a method for making a pharmaceutical package, with a coating or layer between a drug product and the vessel wall, the coating being effective to reduce the rate and/or amount of degradation of the drug product over a period of time, e.g. a shelf-life of the drug product. The present invention also relates to a pharmaceutical package or other vessel and to a method for making a pharmaceutical package with a coating or layer between the contents and the vessel wall, the coating being effective to reduce the rate and/or amount of polysorbate degradation over a period of time, e.g. a shelf-life of a drug solution.

### BACKGROUND OF THE INVENTION

Many drug formulations, and in particular most biologic drug formulations such as monoclonal antibodies (mAb, mAb-A, and mAb-B), include one or more excipients, e.g. a polysorbate as a stabilizer. However, both the active agent of the drug formulation and the one or more excipients used in the drug formulation may degrade over time. That degradation may give rise to a variety of effects, including for example the relatively short shelf-lives of some drug products.

Aspects of the present invention are directed to medical vessels configured to store a liquid drug product containing an active agent and one or more excipients, and having one or more coatings on a drug-contacting surface of the vessel, the one or more coatings being configured and effective to reduce degradation of the drug product (e.g. degradation of the active agent and/or one or more excipients) over time. Example vessels include pre-filled syringes, cartridges, vials, and the like.

Many drug formulations are high concentration liquid protein/peptide solutions, which must maintain stability under storage conditions. However, the high concentration of protein/peptide contained within these drug solutions are prone to intermolecular interactions and aggregation. The proteins/peptides are also subject to surface adhesion and other interactions which increase the propensity of the protein/peptides to form aggregates and particles. Excipients such as polysorbates are commonly used to maintain protein/peptide stability in such solutions. In particular, two polysorbates—PS20 and PS80 (also known as Tween 20 and Tween 80)—have come to be widely used.

Excipient, e.g. polysorbate, degradation during the shelf-life of a drug solution, however, has become a problem. As polysorbate degrades, its stabilizing effect is reduced, leading to increased protein aggregation and associated particle formation. Polysorbate degradation can also directly lead to undesirable particle formation, as the products of polysorbate degradation primarily include free fatty acids, which having low solubility in water and can thus form insoluble particles. Those insoluble particles can also then induce protein aggregation.

Moreover, to account for the reduced amount of stabilizer in a drug solution that results from polysorbate degradation, drug developers and formulators often include significantly more polysorbate in the solution than would be necessary if degradation of the polysorbate could be prevented. For instance, a drug solution may contain at least 3 times more, at least 5 times more, or even up to 10 times more polysorbate than needed as a stabilizer in order to account for degradation of the polysorbate over the shelf-life of a pharmaceutical package. Indeed, polysorbate concentrations are almost always included in solution at a concentration well above the critical micelle concentration.

However, the presence of these high levels of polysorbate in a drug solution may itself be problematic. For instance, there are some patients that have adverse reactions to polysorbate when injected with a polysorbate-containing drug solution.

Aspects of the present invention are directed to medical vessels configured to store a liquid drug solution containing a polysorbate stabilizer, and having one or more coatings on a drug-contacting surface of the vessel, the one or more coatings being configured and effective to reduce the rate and/or amount of polysorbate degradation. Example vessels include pre-filled syringes, vials, and the like. These vessels may enable drug developers to use less polysorbate in a drug formulation.

Other aspects of the present invention are directed to medical vessels configured to store a liquid drug solution containing a polysorbate stabilizer, and having one or more coatings on a drug-contacting surface of the vessel, the one or more coatings being configured and effective to reduce the amount of free fatty acids, such as those formed by polysorbate degradation, present in the drug containing solution over time. Example vessels include pre-filled syringes, vials, and the like. By reducing the amount of free fatty acids, the overall particle content of the drug solution over time, e.g. over the shelf-life of a pharmaceutical package, may be reduced.

Other aspects of the present invention are directed to medical vessels configured to store a liquid drug solution containing a polysorbate stabilizer, and having one or more coatings on a drug-contacting surface of the vessel, the one or more coatings being configured and effective to reduce the amount of protein aggregation particles and/or the rate of protein aggregation in the solution over time. Example vessels include pre-filled syringes, vials, and the like. For instance, by reducing polysorbate degradation, the one or more coatings may be configured and effective to reduce the amount and/or rate of protein aggregation in the solution over time.

Other aspects of the present invention are directed to medical vessels configured to store a liquid drug solution containing a polysorbate stabilizer, and having one or more coatings on a drug-contacting surface of the vessel, the one or more coatings being configured and effective to enable a reduction in the polysorbate concentration of the drug-containing solution introduced into the vessel for storage relative to conventional formulations of the same drug. For instance, where the coating is effective to reduce protein aggregation, e.g. the number of protein aggregate particles, in the solution during storage, the concentration of polysorbate in the initial solution may be reduced relative to that of the same drug-containing solution formulated, e.g., for storage in uncoated glass or plastic.

Although one or more of the coatings described herein may be known in the art to provide the vessel with other beneficial properties, such as improved oxygen barrier properties, improved lubricity, improved hydrophobicity, or the like, the ability of a coating as described herein to reduce the degradation of a drug product, e.g. to reduce the degradation of an active agent and/or excipient, e.g. to reduce the degradation of a polysorbate over time (e.g. over the shelf-life of a drug formulation) and/or to reduce the amount of free fatty acids in a polysorbate-containing drug formulation over time and/or to reduce protein aggregation in a polysorbate-containing drug formulation over time and/or to enable the formulation of drug-containing solutions having significantly reduced concentrations of polysorbates, is a surprising and unexpected result that provides significant new benefits and uses.

### SUMMARY OF THE INVENTION

An aspect of the invention is a drug primary package that includes a vessel having a lumen defined at least in part by a wall and a drug product stored in the lumen. The wall has an interior surface facing the lumen and an outer surface. The vessel also includes a coating or layer that is effective to reduce degradation of the drug product compared to the same vessel without the coating or layer. In some embodiments, the coating or layer may be supported by at least a portion of the interior surface of the wall. In other embodiments, the coating or layer may be supported by one or more elements positioned on or within the lumen, such as one or more microbeads positioned within the lumen, a cap or stopper positioned on or within the lumen, or a plunger positioned within the lumen of a syringe barrel.

In any embodiment, the interior surface of the vessel wall may also include one or more oxygen barrier coatings or layers, one or more water vapor barrier coatings or layers, one or more adhesion, i.e. tie, coatings or layers, one or more pH protective coatings or layers, one or more lubricity coatings or layers, one or more hydrophobic coatings or layers, or any combination thereof.

The coating or layer may be effective to reduce (i) the amount of active agent of the drug product that is degraded relative to the same drug product stored in the same vessel but without the coating, (ii) the amount of one or more excipients of the drug product that is degraded relative to the same drug product stored in the same vessel but without the coating, or (iii) both i and ii. In some embodiments, for instance, the drug product may be stored within the lumen for at least 1 month, optionally at least 2 months, optionally at least 3 months, optionally at least 4 months, optionally at least 5 months, optionally at least 6 months, optionally between 1 month and 12 months, optionally between 1 month and 6 months, optionally 1 month, optionally 3 months, optionally 6 months, optionally 12 months, optionally 18 months, optionally 24 months, optionally 36 months, and the coating or layer may be effective to increase the amount of intact active agent or excipient at the end of the storing by at least 5%, optionally at least 10%, optionally at least 20%, optionally at least 30%, optionally at least 40%, optionally at least 50%, optionally at least 60%, optionally at least 70%, optionally at least 80%, optionally at least 90%, relative to the same drug product stored under the same conditions in the same vessel (for the same length of time) but without the coating.

The coating or layer may also be effective to reduce (i) the rate at which an active agent of the drug product is degraded relative to the same drug product stored in the same vessel but without the coating, (ii) the rate at which one or more excipients of the drug product is degraded relative to the same drug product stored in the same vessel but without the coating, or (iii) both i and ii. In some embodiments, for instance, the coating or layer may be effective to reduce the rate of degradation of the active agent or excipient by at least 5%, optionally at least 10%, optionally at least 20%, optionally at least 30%, optionally at least 40%, optionally at least 50%, optionally at least 60%, optionally at least 70%, optionally at least 80%, optionally at least 90%, relative to the same drug product stored under the same conditions (and for the same length of time) in the same vessel but without the coating.

Another aspect of the invention is a method of reducing the degradation of a drug product. The method includes providing a vessel having a lumen defined at least in part by a wall having an interior surface facing the lumen and an outer surface, applying a coating or layer that is effective to reduce degradation of the drug product to at least a portion of the vessel, and storing a drug product in the lumen of the vessel, such that the drug product is in contact with the coating or layer. In some embodiments, the coating or layer may be applied by PECVD or atomic layer deposition (ALD). In some embodiments, the coating or layer may be applied to at least a portion of the interior surface of the wall and/or to one or more elements positioned on or within the lumen, such as one or more microbeads that are positioned within the lumen, a cap or stopper that is positioned on or within the lumen, or a plunger that is positioned within the lumen of a syringe barrel. Any of a variety of other coatings may also be applied to the interior surface of the vessel wall, including for example one or more oxygen barrier coatings or layers, one or more water vapor barrier coatings or layers, one or more adhesion, i.e. tie, coatings or layers, one or more pH protective coatings or layers, one or more lubricity coatings or layers, one or more hydrophobic coatings or layers, or any combination thereof.

Embodiments of the present method may be effective to reduce (i) the amount of active agent of the drug product that is degraded relative to the same drug product stored in the same vessel but without the coating, (ii) the amount of one or more excipients of the drug product that is degraded relative to the same drug product stored in the same vessel but without the coating, or (iii) both i and ii. In some embodiments, for instance, the drug product may be stored within the lumen for at least 1 month, optionally at least 2 months, optionally at least 3 months, optionally at least 4 months, optionally at least 5 months, optionally at least 6 months, optionally between 1 month and 12 months, optionally between 1 month and 6 months, optionally 1 month, optionally 3 months, optionally 6 months, optionally 12 months, optionally 18 months, optionally 24 months, optionally 36 months, and the coating or layer may increase the amount of intact active agent or excipient at the end of the storing by at least 5%, optionally at least 10%, optionally at least 20%, optionally at least 30%, optionally at least 40%, optionally at least 50%, optionally at least 60%, optionally at least 70%, optionally at least 80%, optionally at least 90%, relative to the same drug product stored under the same conditions in the same vessel (for the same length of time) but without the coating.

Embodiments of the present method may also be effective to reduce (i) the rate at which an active agent of the drug product is degraded relative to the same drug product stored in the same vessel but without the coating, (ii) the rate at which one or more excipients of the drug product is degraded relative to the same drug product stored in the same vessel but without the coating, or (iii) both i and ii. In some embodiments, for instance, the coating or layer may reduce the rate of degradation of the active agent or excipient by at least 5%, optionally at least 10%, optionally at least 20%, optionally at least 30%, optionally at least 40%, optionally at least 50%, optionally at least 60%, optionally at least 70%, optionally at least 80%, optionally at least 90%, relative to the same drug product stored under the same conditions (and for the same length of time) in the same vessel but without the coating.

Another aspect of the invention is a method of reducing the degradation of polysorbate in an aqueous drug-containing solution. The method includes providing a vessel having a lumen defined at least in part by a wall, the wall having an interior surface facing the lumen, with a coating on the interior surface, the coating comprising SiwOxCy or SiwNxCy, where w is 1, x in this formula is from about 0.5 to 2.4, and y is from about 0.6 to about 3. The method further includes storing a drug-containing solution in the lumen of the vessel, such that the drug-containing solution is in contact with the coated interior surface, and with an opening to the lumen being closed by an appropriate means (e.g. stopper, cap, plunger, etc.). It has surprisingly been found that, where the drug-containing solution comprises a polysorbate, the coating may be configured to be effective to reduce the amount of polysorbate that is degraded relative to the same solution stored in the same vessel but without the coating.

An aspect of the invention is a method of preventing the accumulation of polysorbate degradation products in an aqueous drug-containing solution. The method includes providing a vessel having a lumen defined at least in part by a wall, the wall having an interior surface facing the lumen, with a coating on the interior surface, the coating comprising SiwOxCy or SiwNxCy, where w is 1, x in this formula is from about 0.5 to 2.4, and y is from about 0.6 to about 3. The method further includes storing a drug-containing solution in the lumen of the vessel, such that the drug-containing solution is in contact with the coated interior surface, and with an opening to the lumen being closed by an appropriate means (e.g. stopper, cap, plunger, etc.). It has surprisingly been found that, where the drug-containing solution comprises a polysorbate, the coating may be configured to be effective to reduce the amount of one or more polysorbate degradation products that are formed in the solution relative to the same solution stored in the same vessel but without the coating. The polysorbate degradation products may comprise the free fatty acids: caprylic acid, capric acid, lauric acid, myristic acid, linoleic acid, palmitic acid, oleic acid, stearic acid, or any combination thereof.

An aspect of the invention is a method of reducing the degradation of polysorbate in an aqueous drug-containing solution. The method includes providing a vessel having a lumen defined at least in part by a wall, the wall having an interior surface facing the lumen, with a coating on the interior surface, the coating comprising SiwOxCy or SiwNxCy, where w is 1, x in this formula is from about 0.5 to 2.4, and y is from about 0.6 to about 3. The method further includes storing a drug-containing solution in the lumen of the vessel, such that the drug-containing solution is in contact with the coated interior surface, and with an opening to the lumen being closed by an appropriate means (e.g. stopper, cap, plunger, etc.). It has surprisingly been found that, where the drug-containing solution comprises a polysorbate, the coating may be configured to be effective to reduce the rate of polysorbate degradation relative to the same solution stored in the same vessel but without the coating.

An aspect of the invention is the use of a vessel having a lumen defined at least in part by a wall, the wall having an interior surface facing the lumen, and a coating on the interior surface, the coating comprising SiwOxCy or SiwNxCy, where w is 1, x in this formula is from about 0.5 to 2.4, and y is from about 0.6 to about 3, to reduce polysorbate degradation in a drug-containing solution stored within the lumen.

In other aspects of the invention, it is contemplated that additional coatings may be configured and used to reduce polysorbate degradation in a drug-containing solution stored within the lumen of a vessel containing such a coating on the surface that contacts the drug-contacting solution.

For example, an aspect of the invention is a method of reducing the degradation of polysorbate in an aqueous drug composition. The method includes providing a vessel having a lumen defined at least in part by a wall, the wall having an interior surface facing the lumen, and a coating on the interior surface, and storing a drug composition comprising a polysorbate in the lumen of the vessel, such that the drug composition is in contact with the coated interior surface, wherein the coating is effective to reduce the amount of polysorbate that is degraded relative to the same drug composition stored in an otherwise identical vessel but without the coating.

Another aspect of the invention is a method of reducing the degradation of polysorbate in an aqueous drug composition. The method includes providing a vessel having a lumen defined at least in part by a wall, the wall having an interior surface facing the lumen, and a coating on the interior surface, and storing a drug composition comprising a polysorbate in the lumen of the vessel, such that the drug composition is in contact with the coated interior surface, wherein the coating is effective to reduce the amount of one or more polysorbate degradation products that are formed in the drug composition relative to the same solution stored in the same vessel but without the coating. The polysorbate degradation products may comprise the free fatty acids: caprylic acid, capric acid, lauric acid, myristic acid, linoleic acid, palmitic acid, oleic acid, stearic acid, or any combination thereof.

Another aspect of the invention is a method of reducing the degradation of polysorbate in an aqueous drug composition. The method includes providing a vessel having a lumen defined at least in part by a wall, the wall having an interior surface facing the lumen, and a coating on the interior surface, and storing a drug composition comprising a polysorbate in the lumen of the vessel, such that the drug-containing solution is in contact with the coated interior surface, wherein the coating is effective to reduce the rate of polysorbate degradation in the drug composition relative to the same solution stored in the same vessel but without the coating.

Another aspect of the invention is the use of a vessel having a lumen defined at least in part by a wall, the wall having an interior surface facing the lumen, and a coating on the interior surface, the coating being effective to reduce polysorbate degradation within a drug-containing solution stored within the lumen.

Another aspect of the invention is a method of reducing the amount of free fatty acids in an aqueous drug-containing solution. The method includes providing a vessel having a lumen defined at least in part by a wall, the wall having an interior surface facing the lumen, and a coating on the interior surface, and storing a drug-containing solution in the lumen of the vessel, such that the drug-containing solution is in contact with the coated interior surface, wherein the drug-containing solution comprises a polysorbate and the coating is effective to reduce the amount of free fatty acids in the solution relative to the same solution stored in the same vessel but without the coating. In some embodiments, the coating may comprise Si_{w}OₓC_{y} or Si_{w}NₓC_{y}, where w is 1, x is from about 0.5 to 2.4, and y is from about 0.6 to about 3. It has surprisingly been found that, where the drug-containing solution comprises a polysorbate, the coating may be configured to be effective to reduce the amount of one or more free fatty acid polysorbate degradation products that are present in the solution relative to the same solution stored in the same vessel but without the coating. The free fatty acid polysorbate degradation products may comprise caprylic acid, capric acid, lauric acid, myristic acid, linoleic acid, palmitic acid, oleic acid, stearic acid, or any combination thereof.

Another aspect of the invention is a method of reducing the number of particles in an aqueous drug-containing solution. The method includes providing a vessel having a lumen defined at least in part by a wall, the wall having an interior surface facing the lumen, and a coating on the interior surface, and storing a drug-containing solution in the lumen of the vessel, such that the drug-containing solution is in contact with the coated interior surface, wherein the drug-containing solution comprises a polysorbate and the coating is effective to reduce the number of particles comprising free fatty acids in the solution relative to the same solution stored in the same vessel but without the coating. In some embodiments, the coating may comprise Si_{w}OₓC_{y} or Si_{w}NₓC_{y}, where w is 1, x is from about 0.5 to 2.4, and y is from about 0.6 to about 3. It has surprisingly been found that, where the drug-containing solution comprises a polysorbate, the coating may be configured to be effective to reduce the amount of particles comprising one or more free fatty acid polysorbate degradation products that are present in the solution relative to the same solution stored in the same vessel but without the coating. The free fatty acids may comprise caprylic acid, capric acid, lauric acid, myristic acid, linoleic acid, palmitic acid, oleic acid, stearic acid, or any combination thereof.

Another aspect of the invention is the use of a vessel having a lumen defined at least in part by a wall, the wall having an interior surface facing the lumen, and a coating on the interior surface, to reduce the amount of free fatty acids in a drug-containing solution stored within the lumen. In some embodiments, the coating may comprise Si_{w}OₓC_{y} or Si_{w}NₓC_{y}, where w is 1, x is from about 0.5 to 2.4, and y is from about 0.6 to about 3.

Another aspect of the invention is the use of a vessel having a lumen defined at least in part by a wall, the wall having an interior surface facing the lumen, and a coating on the interior surface, to reduce the number of particles comprising free fatty acids in a drug-containing solution stored within the lumen. In some embodiments, the coating may comprise Si_{w}OₓC_{y} or Si_{w}NₓC_{y}, where w is 1, x is from about 0.5 to 2.4, and y is from about 0.6 to about 3. In other aspects of the invention, the reduction in polysorbate degradation and/or free fatty acids, e.g. polysorbate degradation products, may bring about a reduction in the formation of protein aggregates and/or protein aggregate particles in an aqueous drug composition stored within a vessel.

For example, an aspect of the invention is a method of reducing the formation of protein aggregates in an aqueous drug composition. The method includes providing a vessel having a lumen defined at least in part by a wall, the wall having an interior surface facing the lumen, and a coating on the interior surface, and storing a drug composition comprising a protein and a polysorbate in the lumen of the vessel, such that the drug composition is in contact with the coated interior surface, wherein the coating is effective to reduce the amount of protein aggregates formed in the drug composition relative to the same drug composition stored in an otherwise identical vessel but without the coating. In some embodiments, the coating may comprise Si_{w}OₓC_{y} or Si_{w}NₓC_{y}, where w is 1, x is from about 0.5 to 2.4, and y is from about 0.6 to about 3.

Another aspect of the invention is a method of reducing the formation of protein aggregates in an aqueous drug composition. The method includes providing a vessel having a lumen defined at least in part by a wall, the wall having an interior surface facing the lumen, and a coating on the interior surface, and storing a drug composition comprising protein and a polysorbate in the lumen of the vessel, such that the drug composition is in contact with the coated interior surface, wherein the coating is effective to reduce the rate of formation of protein aggregates in the drug composition relative to the same drug composition stored in an otherwise identical vessel but without the coating. In some embodiments, the coating may comprise Si_{w}OₓC_{y} or SiwNxCy, where w is 1, x is from about 0.5 to 2.4, and y is from about 0.6 to about 3.

Another aspect of the invention is the use of a vessel having a lumen defined at least in part by a wall, the wall having an interior surface facing the lumen, and a coating on the interior surface to contain a drug composition comprising a protein and a polysorbate, the coating effective to reduce formation of protein aggregates within the drug composition stored within the lumen. In some embodiments, the coating may comprise Si_{w}OₓC_{y} or Si_{w}NₓC_{y}, where w is 1, x is from about 0.5 to 2.4, and y is from about 0.6 to about 3.

Another aspect of the invention is a method of reducing the formation of protein aggregates in an aqueous drug composition. The method includes providing a vessel having a lumen defined at least in part by a wall, the wall having an interior surface facing the lumen, and a coating on the interior surface, and storing a drug composition comprising a protein and a polysorbate in the lumen of the vessel, such that the drug composition is in contact with the coated interior surface, wherein the coating is effective to reduce the amount of protein aggregate particles formed in the drug composition relative to the same drug composition stored in an otherwise identical vessel but without the coating. In some embodiments, the coating may comprise Si_{w}OₓC_{y} or Si_{w}NₓC_{y}, where w is 1, x is from about 0.5 to 2.4, and y is from about 0.6 to about 3.

Another aspect of the invention is a method of reducing the formation of protein aggregates in an aqueous drug composition. The method includes providing a vessel having a lumen defined at least in part by a wall, the wall having an interior surface facing the lumen, and a coating on the interior surface, and storing a drug composition comprising protein and a polysorbate in the lumen of the vessel, such that the drug composition is in contact with the coated interior surface, wherein the coating is effective to reduce the rate of formation of protein aggregate particles in the drug composition relative to the same drug composition stored in an otherwise identical vessel but without the coating. In some embodiments, the coating may comprise Si_{w}OₓC_{y} or Si_{w}NₓC_{y}, where w is 1, x is from about 0.5 to 2.4, and y is from about 0.6 to about 3.

Another aspect of the invention is the use of a vessel having a lumen defined at least in part by a wall, the wall having an interior surface facing the lumen, and a coating on the interior surface to contain a drug composition comprising a protein and a polysorbate, the coating being effective to reduce formation of protein aggregate particles within the drug composition stored within the lumen. In some embodiments, the coating may comprise Si_{w}OₓC_{y} or Si_{w}NₓC_{y}, where w is 1, x is from about 0.5 to 2.4, and y is from about 0.6 to about 3.

In other aspects of the invention, the reduction in polysorbate degradation and/or polysorbate degradation products (e.g. free fatty acids) and/or the reduction in the formation of protein aggregates and/or protein aggregate particles, in an aqueous drug composition stored within a vessel may enable a reduction in the concentration of polysorbate that is included in the aqeuous drug composition.

For example, an aspect of the present invention is a method of reducing the polysorbate concentration of a drug-containing solution. The method includes providing a vessel having a lumen defined at least in part by a wall, the wall having an interior surface facing the lumen, and a coating on the interior surface, and storing a drug-containing solution in the lumen of the vessel, such that the drug-containing solution is in contact with the coated interior surface, wherein the drug-containing solution comprises a polysorbate stabilizer and the coating is effective to reduce the number of protein aggregate particles in the solution during storage, such that the concentration of polysorbate in the initial solution may be reduced relative to that of a conventional formulation of the same drug composition, for example the drug composition formulated for storage in the same vessel without the coating or the drug composition conventionally formulated for storage in glass. In some embodiments, the coating may comprise Si_{w}OₓC_{y} or Si_{w}NₓC_{y}, where w is 1, x is from about 0.5 to 2.4, and y is from about 0.6 to about 3.

In some embodiments, for example, the concentration of polysorbate in the initial solution, i.e. the solution with which the vessel is filled, may be reduced by at least 10% relative to that of the same drug-containing solution formulated for storage in the same vessel without the coating, optionally reduced by at least 20% relative to that of the same drug-containing solution formulated for storage in the same vessel without the coating, optionally reduced by at least 25% relative to that of the same drug-containing solution formulated for storage in the same vessel without the coating, optionally reduced by at least 30% relative to that of the same drug-containing solution formulated for storage in the same vessel without the coating, optionally reduced by at least 40% relative to that of the same drug-containing solution formulated for storage in the same vessel without the coating, optionally reduced by at least 50% relative to that of the same drug-containing solution formulated for storage in the same vessel without the coating, optionally reduced by at least 60% relative to that of the same drug-containing solution formulated for storage in the same vessel without the coating, optionally reduced by at least 70% relative to that of the same drug-containing solution formulated for storage in the same vessel without the coating, optionally reduced by at least 75% relative to that of the same drug-containing solution formulated for storage in the same vessel without the coating, optionally reduced by at least 80% relative to that of the same drug-containing solution formulated for storage in the same vessel without the coating, optionally reduced by at least 90% relative to that of the same drug-containing solution formulated for storage in the same vessel without the coating.

In some embodiments, for example, the concentration of polysorbate in the initial solution, i.e. the solution with which the vessel is filled, may be reduced by at least 10% relative to that of the same drug-containing solution formulated for storage in glass, optionally reduced by at least 20% relative to that of the same drug-containing solution formulated for storage in glass, optionally reduced by at least 25% relative to that of the same drug-containing solution formulated for storage in glass, optionally reduced by at least 30% relative to that of the same drug-containing solution formulated for storage in glass, optionally reduced by at least 40% relative to that of the same drug-containing solution formulated for storage in glass, optionally reduced by at least 50% relative to that of the same drug-containing solution formulated for storage in glass, optionally reduced by at least 60% relative to that of the same drug-containing solution formulated for storage in glass, optionally reduced by at least 70% relative to that of the same drug-containing solution formulated for storage in glass, optionally reduced by at least 75% relative to that of the same drug-containing solution formulated for storage in glass, optionally reduced by at least 80% relative to that of the same drug-containing solution formulated for storage in glass, optionally reduced by at least 90% relative to that of the same drug-containing solution formulated for storage in glass.

Another aspect of the invention is the use of a vessel having a lumen defined at least in part by a wall, the wall having an interior surface facing the lumen, and a coating on the interior surface, to reduce the concentration of polysorbate in a drug-containing solution that is introduced into the lumen. In some embodiments, the coating may comprise Si_{w}OₓC_{y} or Si_{w}NₓC_{y}, where w is 1, x is from about 0.5 to 2.4, and y is from about 0.6 to about 3.

In other aspects of the invention, any of the above-described methods can be performed by providing the coating on a substrate that contacts the drug-containing solution while the solution is stored within the lumen of a vessel instead of (or in addition to) providing the coating on the vessel wall itself.

For example, an aspect of the present invention is a method of reducing the degradation of polysorbate in an aqueous drug-containing solution. The method includes providing a coating on a substrate, the coating optionally comprising SiwOxCy or SiwNxCy, where w is 1, x in this formula is from about 0.5 to 2.4, and y is from about 0.6 to about 3; placing the coated substrate into the lumen of the vessel or onto the vessel; and storing a drug-containing solution in the lumen of the vessel, such that the drug-containing solution is in contact with the coated substrate; wherein the drug-containing solution comprises a polysorbate and the coating is effective to reduce the amount of polysorbate that is degraded and/or the rate of polysorbate degradation relative to the same solution stored in the same vessel but without the coated substrate.

Another aspect of the present invention is a method of reducing the accumulation of polysorbate degradation products in an aqueous drug-containing solution. The method includes providing a vessel having a lumen; providing a coating on a substrate, the coating optionally comprising SiwOxCy or SiwNxCy, where w is 1, x in this formula is from about 0.5 to 2.4, and y is from about 0.6 to about 3; placing the coated substrate into the lumen of the vessel or onto the vessel; and storing a drug-containing solution in the lumen of the vessel, such that the drug-containing solution is in contact with the coated substrate; wherein the drug-containing solution comprises a polysorbate and the coating is effective to reduce the amount of polysorbate degradation products that are formed in the solution relative to the same solution stored in the same vessel but without the coated substrate.

Another aspect of the present invention is a method of reducing the formation of protein aggregates in an aqueous drug-containing solution. The method includes providing a vessel having a lumen; providing a coating on a substrate, the coating optionally comprising SiwOxCy or SiwNxCy, where w is 1, x in this formula is from about 0.5 to 2.4, and y is from about 0.6 to about 3; placing the coated substrate into the lumen of the vessel or onto the vessel; and storing a drug-containing solution in the lumen of the vessel, such that the drug-containing solution is in contact with the coated substrate; wherein the drug-containing solution comprises a polysorbate and the coating is effective to reduce the amount of protein aggregates formed in the solution and/or the rate of protein aggregate formation and/or the number of protein aggregate particles in the solution relative to the same solution stored in the same vessel but without the coated substrate.

Another aspect of the invention is a method of reducing the amount of free fatty acids and/or the amount of particles in an aqueous drug-containing solution. The method includes providing a vessel having a lumen; providing a coating on a substrate, the coating optionally comprising SiwOxCy or SiwNxCy, where w is 1, x in this formula is from about 0.5 to 2.4, and y is from about 0.6 to about 3; placing the coated substrate into the lumen of the vessel or onto the vessel; and storing a drug-containing solution in the lumen of the vessel, such that the drug-containing solution is in contact with the coated substrate; wherein the drug-containing solution comprises a polysorbate and the coating is effective to reduce the amount of free fatty acids in the solution and/or the amount of particles comprising free fatty acids in the solution relative to the same solution stored in the same vessel but without the coated substrate.

Another aspect of the invention is a method of reducing the polysorbate concentration of a drug-containing solution. The method includes providing a vessel having a lumen; providing a coating on a substrate, the coating optionally comprising SiwOxCy or SiwNxCy, where w is 1, x in this formula is from about 0.5 to 2.4, and y is from about 0.6 to about 3; placing the coated substrate into the lumen of the vessel or onto the vessel; and storing a drug-containing solution in the lumen of the vessel, such that the drug-containing solution is in contact with the coated substrate; wherein the drug-containing solution comprises a polysorbate stabilizer and the coating is effective to reduce the number of protein aggregate particles in the solution during storage, such that the concentration of polysorbate in the initial drug-containing solution is reduced relative to that of the same drug-containing solution formulated for the same vessel but without the coated substrate.

In any of these substrate-containing embodiments, the substrate may be a microbead. For instance, a plurality of coated microbeads may be placed into the vessel, e.g. into a syringe or a vial, without having any negative effect on the functioning of the vessel, e.g. the operation of a syringe. Moreover, the microbeads may be sized so as to be too big to pass through a needle of a syringe. Alternatively, in any of these embodiments, the substrate may be a plunger or a portion of a plunger, such as a plunger head or elastic plunger head, that may be inserted into a syringe barrel. Alternatively, in any of these embodiments, the substrate may be a cap or a portion of a cap, for instance a vial cap. In any of these embodiments, a coated substrate, such as any of those described above, may also be used in combination with a vessel having a coated wall as described herein.

In other aspects of the invention, the coating may be configured and effective to prevent or avoid the leaching of metal ions into the drug-containing solution stored within the lumen. It is contemplated that metal ions be at least partially responsible for polysorbate degradation. Accordingly, the coating may be configured to avoid and/or prevent the leaching of metal ions (also known as ion exchange) from the vessel into the drug-containing solution.

For example, an aspect of the invention is a method of reducing the degradation of polysorbate in an aqueous drug composition. The method includes providing a vessel having a lumen defined at least in part by a wall, the wall having an interior surface, optionally comprising a coating, facing the lumen, in which the interior surface is free or substantially free from metals and/or metal ions, and storing a drug composition comprising a polysorbate in the lumen of the vessel, such that the drug composition is in contact with the interior surface, wherein the interior surface is effective to reduce the amount of polysorbate that is degraded relative to the same drug composition stored in an otherwise identical vessel but in which the interior surface comprises borosilicate glass or uncoated plastic (e.g. COP or COC plastic) from which metal ions may be leached.

Another aspect of the invention is a method of reducing the degradation of polysorbate in an aqueous drug composition. The method includes providing a vessel having a lumen defined at least in part by a wall, the wall having an interior surface, optionally comprising a coating, facing the lumen, in which the interior surface is free or substantially free from metals and/or metal ions, and storing a drug composition comprising a polysorbate in the lumen of the vessel, such that the drug composition is in contact with the interior surface, wherein the interior surface is effective to reduce the amount of one or more polysorbate degradation products that are formed in the drug composition relative to the same solution stored in an otherwise identical vessel but in which the interior surface comprises borosilicate glass or uncoated plastic (e.g. COP or COC plastic) from which metal ions may be leached. The polysorbate degradation products may comprise the free fatty acids: caprylic acid, capric acid, lauric acid, myristic acid, linoleic acid, palmitic acid, oleic acid, stearic acid, or any combination thereof.

Another aspect of the invention is a method of reducing the degradation of polysorbate in an aqueous drug composition. The method includes providing a vessel having a lumen defined at least in part by a wall, the wall having an interior surface, optionally comprising a coating, facing the lumen, in which the interior surface is free or substantially free from metals and/or metal ions, and storing a drug composition comprising a polysorbate in the lumen of the vessel, such that the drug composition is in contact with the interior surface, wherein the interior surface is effective to reduce the rate of polysorbate degradation in the drug composition relative to the same solution stored in an otherwise identical vessel but in which the interior surface comprises borosilicate glass or uncoated plastic (e.g. COP or COC plastic) from which metal ions may be leached.

Another aspect of the invention is the use of a vessel having a lumen defined at least in part by a wall, the wall having an interior surface facing the lumen, the interior surface optionally comprising a coating, and the interior surface being effective to reduce polysorbate degradation within a drug-containing solution stored within the lumen (e.g. the amount of polysorbate degraded within a defined storage time and/or the rate of polysorbate degradation within a defined storage time and/or the amount of polysorbate degradation products within a defined storage time).

In some embodiments, the coating may be effective to increase the amount of intact polysorbate at the end of the shelf-life of the solution by at least 5% relative to the same solution stored in a borosilicate glass or uncoated plastic (e.g. COP or COC plastic) vessel from which metal ions may be leached under the same conditions, optionally at least 10%, optionally at least 20%, optionally at least 30%, optionally at least 40%, optionally at least 50%, optionally at least 60%, optionally at least 70%, optionally at least 80%, optionally at least 90%.

In some embodiments, the amount of intact polysorbate after 1 month of storage, optionally after 6 months, optionally after 12 months, optionally after 18 months, optionally after 24 months, optionally after 36 months, may be increased by at least 5%, optionally at least 10%, optionally at least 20%, optionally at least 30%, optionally at least 40%, optionally at least 50%, optionally at least 60%, optionally at least 70%, optionally at least 80%, optionally at least 90%, relative to the same solution stored in a borosilicate glass or uncoated plastic (e.g. COP or COC plastic) vessel from which metal ions may be leached under the same conditions and for the same length of time.

In some embodiments, the rate of degradation of polysorbate may be reduced by at least 5% relative to the same solution being stored in a borosilicate glass vessel under the same conditions, optionally at least 10%, optionally at least 20%, optionally at least 30%, optionally at least 40%, optionally at least 50%, optionally at least 60%, optionally at least 70%, optionally at least 80%, optionally at least 90%.

Another aspect of the invention is a method of reducing the formation of protein aggregates in an aqueous drug composition that comprises a polysorbate stabilizer. The method includes providing a vessel having a lumen defined at least in part by a wall, the wall having an interior surface, optionally comprising a coating, facing the lumen, in which the interior surface is free or substantially free from metals and/or metal ions, and storing a drug composition comprising a polysorbate in the lumen of the vessel, such that the drug composition is in contact with the interior surface, wherein the interior surface is effective to reduce the amount of protein aggregates formed in the drug composition relative to the same solution stored in an otherwise identical vessel but in which the interior surface comprises borosilicate glass or uncoated plastic (e.g. COP or COC plastic) from which metal ions may be leached. The protein aggregates may be the result of polysorbate degradation.

Another aspect of the invention is a method of reducing the formation of protein aggregates in an aqueous drug composition that comprises a polysorbate stabilizer. The method includes providing a vessel having a lumen defined at least in part by a wall, the wall having an interior surface, optionally comprising a coating, facing the lumen, in which the interior surface is free or substantially free from metals and/or metal ions, and storing a drug composition comprising a polysorbate in the lumen of the vessel, such that the drug composition is in contact with the interior surface, wherein the interior surface is effective to reduce the rate of formation of protein aggregates in the drug composition relative to the same solution stored in an otherwise identical vessel but in which the interior surface comprises borosilicate glass or uncoated plastic (e.g. COP or COC plastic) from which metal ions may be leached. The protein aggregates may be the result of polysorbate degradation.

Another aspect of the invention is the use of a vessel having a lumen defined at least in part by a wall, the wall having an interior surface facing the lumen, the interior surface optionally comprising a coating, and the interior surface being effective to reduce the formation of protein aggregates within a drug-containing solution stored within the lumen (e.g. the amount of protein aggregates formed within a defined storage time and/or the rate of formation of protein aggregates within a defined storage time), in which the drug-containing solution comprises a protein and a polysorbate stabilizer. The protein aggregates may be the result of polysorbate degradation.

Another aspect of the invention is a method of reducing protein aggregation in a pharmaceutical package comprising an aqueous drug composition that comprises a polysorbate stabilizer. The method includes providing a vessel having a lumen defined at least in part by a wall, the wall having an interior surface, optionally comprising a coating, facing the lumen, in which the interior surface is free or substantially free from metals and/or metal ions, and storing a drug composition comprising a polysorbate in the lumen of the vessel, such that the drug composition is in contact with the interior surface, wherein the interior surface is effective to reduce the number of protein aggregate particles in the drug composition relative to the same composition stored in an otherwise identical vessel but in which the interior surface comprises borosilicate glass or uncoated plastic (e.g. COP or COC plastic) from which metal ions may be leached. The protein aggregate particles may be the result of polysorbate degradation.

Another aspect of the invention is the use of a vessel having a lumen defined at least in part by a wall, the wall having an interior surface facing the lumen, the interior surface optionally comprising a coating, and the interior surface being effective to reduce the formation of protein aggregate particles within a drug-containing solution stored within the lumen (e.g. the amount of protein aggregate particles formed within a defined storage time and/or the rate of formation of protein aggregate particles within a defined storage time), in which the drug-containing solution comprises a protein and a polysorbate stabilizer. The protein aggregate particles may be the result of polysorbate degradation.

Another aspect of the invention is a method of reducing free fatty acids in a pharmaceutical package comprising an aqueous drug composition that comprises a polysorbate stabilizer. The method includes providing a vessel having a lumen defined at least in part by a wall, the wall having an interior surface, optionally comprising a coating, facing the lumen, in which the interior surface is free or substantially free from metals and/or metal ions, and storing a drug composition comprising a polysorbate in the lumen of the vessel, such that the drug composition is in contact with the interior surface, wherein the interior surface is effective to reduce the amount of free fatty acids in the drug composition relative to the same composition stored in an otherwise identical vessel but in which the interior surface comprises borosilicate glass or uncoated plastic (e.g. COP or COC plastic) from which metal ions may be leached. The free fatty acids may be the result of polysorbate degradation.

Another aspect of the invention is a method of reducing the amount of particles in a pharmaceutical package comprising an aqueous drug composition that comprises a polysorbate stabilizer. The method includes providing a vessel having a lumen defined at least in part by a wall, the wall having an interior surface, optionally comprising a coating, facing the lumen, in which the interior surface is free or substantially free from metals and/or metal ions, and storing a drug composition comprising a polysorbate in the lumen of the vessel, such that the drug composition is in contact with the interior surface, wherein the interior surface is effective to reduce the number of particles comprising free fatty acids in the drug composition relative to the same composition stored in an otherwise identical vessel but in which the interior surface comprises borosilicate glass or uncoated plastic (e.g. COP or COC plastic) from which metal ions may be leached. The particles may be the result of polysorbate degradation.

Another aspect of the invention is the use of a vessel having a lumen defined at least in part by a wall, the wall having an interior surface facing the lumen, the interior surface optionally comprising a coating, and the interior surface being effective to reduce the amount of free fatty acids within a drug-containing solution stored within the lumen (e.g. the amount of free fatty acids formed within a defined storage time and/or the rate of formation of free fatty acids within a defined storage time), in which the drug-containing solution comprises a protein and a polysorbate stabilizer. The free fatty acids may be the result of polysorbate degradation.

Another aspect of the invention is the use of a vessel having a lumen defined at least in part by a wall, the wall having an interior surface facing the lumen, the interior surface optionally comprising a coating, and the interior surface being effective to reduce the amount of particles within a drug-containing solution stored within the lumen (e.g. the amount of particles comprising free fatty acids formed within a defined storage time and/or the rate of formation particles comprising free fatty acids within a defined storage time), in which the drug-containing solution comprises a protein and a polysorbate stabilizer. The particles may be the result of polysorbate degradation.

In some embodiments, at the end of the storing the number of protein aggregate particles in the drug-containing solution may be reduced by at least 10% relative to the same solution stored in a borosilicate glass or uncoated plastic (e.g. COP or COC plastic) vessel from which metal ions may be leached, optionally at least 20%, optionally at least 25%, optionally at least 30%, optionally at least 40%, optionally at least 50%, optionally at least 60%, optionally at least 70%, optionally at least 75%, optionally at least 80%, optionally at least 90%.

In some embodiments, the coating may be effective to reduce the number of protein aggregate particles at the end of the shelf-life of the pharmaceutical package by at least 5% relative to the same solution stored in a borosilicate glass or uncoated plastic (e.g. COP or COC plastic) vessel from which metal ions may be leached under the same conditions, optionally at least 10%, optionally at least 20%, optionally at least 30%, optionally at least 40%, optionally at least 50%, optionally at least 60%, optionally at least 70%, optionally at least 80%, optionally at least 90%.

In some embodiments, the number of protein aggregate particles after 1 month of storage, optionally after 6 months, optionally after 12 months, optionally after 18 months, optionally after 24 months, optionally after 36 months, is reduced by at least 5%, optionally at least 10%, optionally at least 20%, optionally at least 30%, optionally at least 40%, optionally at least 50%, optionally at least 60%, optionally at least 70%, optionally at least 80%, optionally at least 90%, relative to the same solution stored in a borosilicate glass or uncoated plastic (e.g. COP or COC plastic) vessel from which metal ions may be leached under the same conditions and for the same length of time.

In other aspects of the invention, the reduction in polysorbate degradation and/or polysorbate degradation products (e.g. free fatty acids), and/or the reduction in the formation of protein aggregates and/or protein aggregate particles, in an aqueous drug composition stored within a vessel may enable a reduction in the concentration of polysorbate that is included in the aqeuous drug composition.

For example, an aspect of the present invention is a method of reducing the polysorbate concentration of a drug-containing solution. The method includes providing a vessel having a lumen defined at least in part by a wall, the wall having an interior surface, optionally comprising a coating, facing the lumen, in which the interior surface is free or substantially free from metals and/or metal ions, and storing a drug composition comprising a polysorbate in the lumen of the vessel, such that the drug composition is in contact with the interior surface, wherein the interior surface is effective to reduce polysorbate degradation in the solution during storage, such that the concentration of polysorbate in the initial solution may be reduced relative to that of a conventional formulation of the same drug composition, for example the drug composition formulated for storage in the same vessel but in which the interior surface comprises borosilicate glass or uncoated plastic (e.g. COP or COC plastic) from which metal ions may be leached.

In some embodiments, for example, the concentration of polysorbate in the initial solution, i.e. the solution with which the vessel is filled, may be reduced by at least 10% relative to that of the same drug-containing solution formulated for storage in borosilicate glass or uncoated plastic (e.g. COP or COC plastic) from which metal ions may be leached, optionally reduced by at least 20%, optionally reduced by at least 25%, optionally reduced by at least 30%, optionally reduced by at least 40%, optionally reduced by at least 50%, optionally reduced by at least 60%, optionally reduced by at least 70%, optionally reduced by at least 75%, optionally reduced by at least 80%, optionally reduced by at least 90%.

In any of the preceding embodiments, the storing may take place for at least 1 month, optionally at least two months, optionally at least 3 months, optionally at least 4 months, optionally at least 5 months, optionally at least 6 months, optionally between 1 month and 12 months, optionally between 1 month and 6 months, optionally 1 month, optionally 3 months, optionally 6 months, optionally 9 months, optionally 12 months.

In any of the preceding embodiments, the SiwOxCy coating may be formed by PECVD using a feed gas that comprises a siloxane precursor; optionally a linear siloxane precursor or a monocyclic siloxane precursor or a polycyclic siloxane precursor, optionally a linear siloxane precursor, optionally a monocyclic siloxane precursor, optionally a polycyclic siloxane precursor, optionally an OMCTS precursor.

In any of the preceding embodiments, the coating may also operate as a lubricity coating as described herein.

In any of the preceding embodiments, the coating may have a thickness of 10 to 500 nm, optionally 10 to 200 nm, optionally 20 to 100 nm.

In any of the preceding embodiments, the coating may further comprise a trilayer coating comprising:
a tie coating or layer comprising SiOₓC_{y} or SiNₓC_{y} wherein x is from about 0.5 to about 2.4 and y is from about 0.6 to about 3, the tie coating or layer having an interior surface facing the lumen and an outer surface facing the wall interior surface;
a barrier coating or layer being from 2 to 1000 nm thick and comprising SiOₓ, wherein x is from 1.5 to 2.9, the barrier coating or layer of SiOₓ having an interior surface facing the lumen and an outer surface facing the interior surface of the tie coating or layer, the barrier coating or layer being effective to reduce the ingress of atmospheric gas into the lumen compared to a vessel without a barrier coating or layer;
a pH protective coating or layer comprising SiOₓC_{y} or SiNₓC_{y} wherein x is from about 0.5 to about 2.4 and y is from about 0.6 to about 3, the pH protective coating or layer having an interior surface facing the lumen and an outer surface facing the interior surface of the barrier coating or layer.

In any of the preceding embodiments, the vessel may be a syringe or a vial, optionally a glass syringe or vial, optionally a plastic syringe or vial, optionally made of a cyclic olefin polymer (COP), cyclic block co-polymer (CBC), or cyclic olefin copolymer (COC).

In any of the preceding embodiments, the coating may be effective to increase the amount of intact polysorbate at the end of the shelf-life of the drug-containing solution by at least 5% relative to the same solution stored in an uncoated vessel under the same conditions, optionally at least 10%, optionally at least 20%, optionally at least 30%, optionally at least 40%, optionally at least 50%, optionally at least 60%, optionally at least 70%, optionally at least 80%, optionally at least 90%.

In any of the preceding embodiments, the coating may be effective such that the amount of intact polysorbate after 1 month of storage, optionally after 6 months, optionally after 12 months, optionally after 18 months, optionally after 24 months, optionally after 36 months, may be increased by at least 5%, optionally at least 10%, optionally at least 20%, optionally at least 30%, optionally at least 40%, optionally at least 50%, optionally at least 60%, optionally at least 70%, optionally at least 80%, optionally at least 90%, relative to the same solution stored in an uncoated vessel under the same conditions and for the same length of time.

In any of the preceding embodiments, the coating may be effective such that the rate of degradation of polysorbate may be reduced by at least 5% relative to the same solution being stored in an uncoated vessel under the same conditions, optionally at least 10%, optionally at least 20%, optionally at least 30%, optionally at least 40%, optionally at least 50%, optionally at least 60%, optionally at least 70%, optionally at least 80%, optionally at least 90%.

In any of the preceding embodiments, the polysorbate may be selected from the group consisting of polysorbate 80 and polysorbate 20, may be polysorbate 80, or may be polysorbate 20.

In any of the preceding embodiments, the solution may comprise a protein-based drug.

In any of the preceding embodiments, the solution may comprise a monoclonal antibody selected from the group consisting of mAb, mAb-A, and mAb-B.

In any of the preceding embodiments, the solution may comprise a drug selected from the group consisting of IgG1 mAb, IgG4 mAb, Fab, scFv, nanobodies, nanobody monomers, nanobody dimers, nanobody trimers, nanobody tetramers, nanobody pentamers, nanobody dimer-PEG, nanobody dimer-HSA, Bispecific T-cell engagers (BiTE), and Dual-Affinity ReTargeting antibodies (DART).

In any of the preceding embodiments, the drug-containing solution may be stored in the vessel lumen at 2-8° C.

In any of the preceding embodiments, the initial concentration of polysorbate in the solution may be between 0.01 mg/mL and 1 mg/mL, alternatively between 0.01 mg/mL and 0.9 mg/mL, alternatively between 0.01 mg/mL and 0.8 mg/mL, alternatively between 0.01 mg/mL and 0.7 mg/mL, alternatively between 0.01 mg/mL and 0.6 mg/mL, alternatively between 0.01 mg/mL and 0.5 mg/mL, alternatively between 0.01 mg/mL and 0.4 mg/mL, alternatively between 0.01 mg/mL and 0.3 mg/mL, alternatively between 0.01 mg/mL and 0.2 mg/mL, alternatively between 0.01 mg/mL and 0.1 mg/mL, alternatively between 0.01 mg/mL and 0.09 mg/mL, alternatively between 0.01 mg/mL and 0.08 mg/mL, alternatively between 0.01 mg/mL and 0.07 mg/mL.

In any of the preceding embodiments, the solution may initially contain between 0.01 and 0.09% w/v polysorbate, alternatively between 0.01% and 0.08% w/v polysorbate, alternatively between 0.01% and 0.06% w/v polysorbate, alternatively between 0.01% and 0.05% w/v polysorbate, alternatively between 0.01% and 0.04% w/v polysorbate, alternatively between 0.02% and 0.08% w/v polysorbate, alternatively between 0.02% and 0.07% w/v polysorbate, alternatively between 0.02% and 0.06% w/v polysorbate, alternatively between 0.02% and 0.05% w/v polysorbate, alternatively between 0.02% and 0.04% w/v polysorbate.

In any of the preceding embodiments, the coating may be effective such that after six months of storage at 5° C., the concentration of the polysorbate in the solution is reduced by less than 10%, alternatively less than 9%, alternatively less than 8%, alternatively less than 7%, alternatively less than 6%, alternatively less than 5%, alternatively less than 4%, alternatively less than 3%, alternatively less than 2%, alternatively less than 1%.

In any of the preceding embodiments, the coating may be effective such that at the end of the shelf-life of the drug-containing solution, less than 20%, optionally less than 18%, optionally less than 16%, optionally less than 15%, optionally less than 14%, optionally less than 12%, optionally less than 10%, optionally less than 8%, optionally less than 6%, optionally less than 5%, optionally less than 4%, optionally less than 3%, optionally less than 2%, of the initial polysorbate in the solution has degraded.

In any of the preceding embodiments, at least 90% of the polysorbate, more desirably at least 95% of the polysorbate, more desirably at least 97% of the polysorbate, more desirably at least 99% of the polysorbate, may remain present in the solution after storage in the coated vessel for 3 months at 25° C.

In any of the preceding embodiments, at least 90% of the polysorbate, more desirably at least 95% of the polysorbate, more desirably at least 97% of the polysorbate, more desirably at least 99% of the polysorbate, may remain present in the solution after storage in the coated vessel for 1 months at 40° C. In any of the preceding embodiments, the coating may be effective so that after storage for 3 months at 25° C., substantially no free fatty acid polysorbate degradation products are present in the solution, i.e. the solution is substantially free of free fatty acid polysorbate degradation products. In any of the preceding embodiments, the coating may be effective so that after storage for 1 months at 40° C., substantially no free fatty acid polysorbate degradation products are present in the solution, i.e. the solution is substantially free of free fatty acid polysorbate degradation products.

In any of the preceding embodiments, the coating may be effective so that after storage for 3 months at 25° C., substantially no particles comprising free fatty acid polysorbate degradation products are present in the solution, i.e. the solution is substantially free of particles comprising free fatty acid polysorbate degradation products.

In any of the preceding embodiments, the coating may be effective so that after storage for 1 months at 40° C., substantially no particles comprising free fatty acid polysorbate degradation products are present in the solution, i.e. the solution is substantially free of particles comprising free fatty acid polysorbate degradation products.

In any of the preceding embodiments, the storing may take place for at least 1 month, optionally at least 2 months, optionally at least 3 months, optionally at least 4 months, optionally at least 5 months, optionally at least 6 months, optionally between 1 month and 12 months, optionally between 1 month and 6 months, optionally 1 month, optionally 3 months, optionally 6 months. Further, in any of the preceding embodiments, at the end of the storing, the number of protein aggregate particles in the drug-containing solution may be reduced by at least 10% relative to the same solution stored in the same vessel but without the coating, optionally at least 20%, optionally at least 25%, optionally at least 30%, optionally at least 40%, optionally at least 50%, optionally at least 60%, optionally at least 70%, optionally at least 75%, optionally at least 80%, optionally at least 90%.

In any of the preceding embodiments, the coating may be effective to reduce the number of protein aggregate particles at the end of the shelf-life of the pharmaceutical package by at least 5% relative to the same solution stored in the same vessel without the coating under the same conditions, optionally at least 10%, optionally at least 20%, optionally at least 30%, optionally at least 40%, optionally at least 50%, optionally at least 60%, optionally at least 70%, optionally at least 80%, optionally at least 90%.

In any of the preceding embodiments, the number of protein aggregate particles after 1 month of storage, optionally after 6 months, optionally after 12 months, optionally after 18 months, optionally after 24 months, optionally after 36 months, may be reduced by at least 5%, optionally at least 10%, optionally at least 20%, optionally at least 30%, optionally at least 40%, optionally at least 50%, optionally at least 60%, optionally at least 70%, optionally at least 80%, optionally at least 90%, relative to the same solution stored in the same vessel without the coating under the same conditions and for the same length of time.

In any of the preceding embodiments, the reduction in polysorbate degradation and/or the reduction in free fatty acids in the solution, and/or the reduction in particles comprising free fatty acids in the solution, and/or the reduction in protein aggregates in the solution achieved by the coating allows for a reduction in the initial concentration of polysorbate in the drug-containing solution. In some embodiments, the coating may be configured and effective to provide at least a 10% reduction in the initial concentration of polysorbate in the drug-containing solution, optionally at least a 20% reduction in the initial concentration of polysorbate in the drug-containing solution, optionally at least a 25% reduction in the initial concentration of polysorbate in the drug-containing solution, optionally at least a 30% reduction in the initial concentration of polysorbate in the drug-containing solution, optionally at least a 40% reduction in the initial concentration of polysorbate in the drug-containing solution, optionally at least a 50% reduction in the initial concentration of polysorbate in the drug-containing solution, optionally at least a 60% reduction in the initial concentration of polysorbate in the drug-containing solution, optionally at least a 70% reduction in the initial concentration of polysorbate in the drug-containing solution, optionally at least a 75% reduction in the initial concentration of polysorbate in the drug-containing solution, optionally at least an 80% reduction in the initial concentration of polysorbate in the drug-containing solution, optionally at least a 90% reduction in the initial concentration of polysorbate in the drug-containing solution.

Many additional and alternative aspects and embodiments of the invention are also contemplated, and are described in the specification and claims that follow. Some optional features contemplated for any of the embodiments of FIGS. 1-5 include the following: A vessel as previously described is contemplated in any embodiment, in which at least a portion of the wall of the vessel comprises a cyclic olefin polymer (COP). A vessel as previously described is contemplated in any embodiment, in which at least a portion of the wall of the vessel comprises a cyclic block co-polymer (CBC). A vessel as previously described is contemplated in any embodiment, in which at least a portion of the wall of the vessel comprises a cyclic olefin copolymer (COC). A vessel as previously described is contemplated in any embodiment, in which at least a portion of the wall of the vessel comprises a glass material, a metal, a ceramic, a composite material, a plastic material, or any combination thereof.

A vessel as previously described is contemplated in any embodiment, comprising a syringe barrel, a cartridge, a vial, or a blister package.

A vessel as previously described is contemplated in any embodiment, in which the fluid comprises a member selected from the group consisting of:

### Biologic Drugs

abatacept; abciximab; abobotulinumtoxinA; adalimumab; adalimumab-adaz; adalimumab-adbm; adalimumab-afzb; adalimumab-atto; adalimumab-bwwd; ado-trastuzumab emtansine; aflibercept; agalsidase beta; albiglutide; albumin chromated CR-51 serum; aldesleukin; alefacept; alemtuzumab; alglucosidase alfa; alirocumab; alteplase; anakinra; aprotinin; asfotas alfa; asparaginase; asparaginase Erwinia chrysanthemi; atezolizumab; avelumab; basiliximab; becaplermin; belatacept; belimumab; benralizumab; beractant; bevacizumab; bevacizumab-awwb; bevacizumab-bvzr; bezlotoxumab; blinatumomab; brentuximab vedotin; brodalumab; brolucizumab-dbll; burosumab-twza; calaspargase pegol-mknl; calfactant; canakinumab; caplacizumab-yhdp; capromab pendetide; cemiplimab-rwic; cenegermin-bkbj; cerliponase alfa; certolizumab pegol; cetuximab; choriogonadotropin alfa; chorionic gonadotropin; chymopapain; collagenase; collagenase clostridium histolyticum; corticorelin ovine triflutate; crizanlizumab-tmca; daclizumab; daratumumab; daratumumab and hyaluronidase-fihj; darbepoetin alpha; denileukin diftitox; denosumab; desirudin; dinutuximab; domase alfa; drotrecogin alfa; dulaglutide; dupilumab; durvalumab; ecallantide; eculizumab; efalizumab; elapegademase-lvlr; elosulfase alfa; elotuzumab; emapalumab-lzsg; emicizumab-kxwh; enfortumab vedotin-ejfv; epoetin alfa; epoetin alfa-epbx; erenumab-aooe; etanercept; etanercept-szzs; etanercept-ykro; evolocumab; fam-trastuzumab deruxetecan-nxki; fibrinolysin and desoxyribonuclease combined [bovine], with chloramphenicol; filgrastim; filgrastim-aafi; filgrastim-sndz; follitropin alfa; follitropin beta; fremanezumab-vfrm; galcanezumab-gnlm; galsulfase; gemtuzumab ozogamicin; glucarpidase; golimumab; guselkumab; hyaluronidase; hyaluronidase human; ibalizumab-uiyk; ibritumomab tiuxetan; idarucizumab; idursulfase; imiglucerase; incobotulinumtoxinA; inebilizumab-cdon; infliximab; infliximab-abda; infliximab-axxq; infliximab-dyyb; infliximab-qbtx; inotuzumab ozogamicin; insulin aspart; insulin aspart protamine and insulin aspart; insulin degludec; insulin degludec and insulin aspart; insulin degludec and liraglutide; insulin detemir; insulin glargine; insulin glargine and lixisenatide; insulin glulisine; insulin human; insulin isophane human; insulin isophane human and insulin human; insulin lispro; insulin lispro protamine and insulin lispro; insulin lispro-aabc; interferon alfa-2a; interferon alfa-2b; interferon alfacon-1; interferon alfa-n3 (human leukocyte derived); interferon beta-1a; interferon beta-1b; interferon gamma-1b; ipilimumab; isatuximab-irfc; ixekizumab; lanadelumab-flyo; laronidase; lixisenatide; luspatercept-aamt; mecasermin; mecasermin rinfabate; menotropins; mepolizumab; methoxy polyethylene glycol-epoetin beta; metreleptin; mogamulizumab-kpkc; moxetumomab pasudotox-tdfk; muromanab-CD3; natalizumab; necitumumab; nivolumab; nofetumomab; obiltoxaximab; obinutuzumab; ocrelizumab; ocriplasmin; ofatumumab; olaratumab; omalizumab; onabotulinumtoxinA; oprelvekin; palifermin; palivizumab; pancrelipase; panitumumab; parathyroid hormone; pegademase bovine; pegaspargase; pegfilgrastim; pegfilgrastim-apgf; pegfilgrastim-bmez; pegfilgrastim-cbqv; pegfilgrastim-jmdb; peginterferon alfa-2a; peginterferon alfa-2a and ribavirin; peginterferon alfa-2b; peginterferon alfa-2b and ribavirin; peginterferon beta-1a; pegloticase; pegvaliase-pqpz; pegvisomant; pembrolizumab; pertuzumab; polatuzumab vedotin-piiq; poractant alfa; prabotulinumtoxinA-xvfs; radiolabeled albumin technetium Tc-99m albumin colloid kit; ramucirumab; ranibizumab; rasburicase; ravulizumab-cwvz; raxibacumab; reslizumab; reteplase; rilonacept; rimabotulinumtoxinB; risankizumab-rzaa; rituximab; rituximab and hyaluronidase human; rituximab-abbs; rituximab-pvvr; romiplostim; romosozumab-aqqg; sacituzumab govitecan-hziy; sacrosidase; sargramostim; sarilumab; sebelipase alfa; secukinumab; siltuximab; somatropin; tagraxofusp-erzs; taliglucerase alfa; tbo-filgrastim; technetium 99m tc fanolesomab; tenecteplase; teprotumumab-trbw; tesamorelin acetate; thyrotropin alfa; tildrakizumab-asmn; tocilizumab; tositumomab and iodine I-131 tositumomab; trastuzumab; trastuzumab and hyaluronidase-oysk; trastuzumab-anns; trastuzumab-dkst; trastuzumab-dttb; trastuzumab-pkrb; trastuzumab-qyyp; urofollitropin; urokinase; ustekinumab; vedolizumab; velaglucerase alfa; vestronidase alfa-vjbk; Ziv-Aflibercept; Amjevita (adalimumab-atto); Dupixent (dupilumab); Fulphila (pegfilgrastim-jmdb); Ilaris (canakinumab); Ixifi (infliximab-qbtx); Lyumjev (insulin lispro-aabc); Nyvepria (pegfilgrastim-apgf); Ogivri (trastuzumab-dkst); Semglee (insulin glargine); Uplizna (inebilizumab-cdon); A.P.L. (chorionic gonadotropin); Abrilada (adalimumab-afzb); Accretropin (somatropin); Actemra (tocilizumab); Acthrel (corticorelin ovine triflutate); Actimmune (interferon gamma-1b); Activase (alteplase); Adagen (pegademase bovine); Adakveo (crizanlizumab-tmca); Adcetris (brentuximab vedotin); Adlyxin (lixisenatide); Admelog (insulin lispro); Afrezza (insulin human); Aimovig (erenumab-aooe); Ajovy (fremanezumab-vfrm); Aldurazyme (laronidase); Alferon N Injection (interferon alfa-n3 (human leukocyte derived)); Amevive (alefacept); Amphadase (hyaluronidase); Anthim (obiltoxaximab); Apidra (insulin glulisine); Aranesp (darbepoetin alpha); Arcalyst (rilonacept); Arzerra (ofatumumab); Asparlas (calaspargase pegol-mknl); Avastin (bevacizumab); Avonex (interferon beta-1a); Avsola (infliximab-axxq); Basaglar (insulin glargine); Bavencio (avelumab); Benlysta (belimumab); Beovu (brolucizumab-dbll); Besponsa (inotuzumab ozogamicin); Betaseron (interferon beta-1b); Bexxar (tositumomab and iodine I-131 tositumomab); Blincyto (blinatumomab); Botox (onabotulinumtoxinA); Botox Cosmetic (onabotulinumtoxinA); Bravelle (urofollitropin); Brineura (cerliponase alfa); Cablivi (caplacizumab-yhdp); Campath (alemtuzumab); Cathflo Activase (alteplase); Cerezyme (imiglucerase); Chorionic Gonadotropin (chorionic gonadotropin); Chromalbin (albumin chromated CR-51 serum); Chymodiactin (chymopapain); Cimzia (certolizumab pegol); Cinqair (reslizumab); Cosentyx (secukinumab); Cotazym (pancrelipase); Creon (pancrelipase); Crysvita (burosumab-twza); Curosurf (poractant alfa); Cyltezo (adalimumab-adbm); Cyramza (ramucirumab); Darzalex (daratumumab); Darzalex Faspro (daratumumab and hyaluronidase-fihj); Draximage MAA (kit for the preparation of technetium Tc-99m albumin aggregated); Dysport (abobotulinumtoxinA); Egrifta (tesamorelin acetate); Egrifta SV (tesamorelin acetate); Elaprase (idursulfase); Elase-chloromycetin (fibrinolysin and desoxyribonuclease combined [bovine], with chloramphenicol); Elelyso (taliglucerase alfa); Elitek (rasburicase); Elspar (asparaginase); Elzonris (tagraxofusp-erzs); Emgality (galcanezumab-gnlm); Empliciti (elotuzumab); Enbrel (etanercept); Enbrel Mini (etanercept); Enhertu (fam-trastuzumab deruxetecan-nxki); Entyvio (vedolizumab); Epogen/Procrit (epoetin alfa); Erbitux (cetuximab); Erelzi (etanercept-szzs); Erelzi Sensoready (etanercept-szzs); Erwinaze (asparaginase Erwinia chrysanthemi); Eticovo (etanercept-ykro); Evenity (romosozumab-aqqg); Extavia (interferon beta-1b); Eylea (aflibercept); Fabrazyme (agalsidase beta); Fasenra (benralizumab); Fiasp (insulin aspart); Follistim (follitropin beta); Follistim AQ (follitropin beta); Follistim AQ Cartridge (follitropin beta); Gamifant (emapalumab-lzsg); Gazyva (obinutuzumab); Genotropin (somatropin); Gonal-f (follitropin alfa); Gonal-f RFF (follitropin alfa); Gonal-f RFF RediJect (follitropin alfa); Granix (tbo-filgrastim); Hadlima (adalimumab-bwwd); Hemlibra (emicizumab-kxwh); Herceptin (trastuzumab); Herceptin Hylecta (trastuzumab and hyaluronidase-oysk); Herzuma (trastuzumab-pkrb); Humalog (insulin lispro); Humalog Mix 50/50 (insulin lispro protamine and insulin lispro); Humalog Mix 75/25 (insulin lispro protamine and insulin lispro); Humatrope (somatropin); Humegon (menotropins); Humira (adalimumab); Humulin 70/30 (insulin isophane human and insulin human); Humulin N (insulin isophane human); Humulin R U-100 (insulin human); Humulin R U-500 (insulin human); Hydase (hyaluronidase); Hylenex recombinant (hyaluronidase human); Hyrimoz (adalimumab-adaz); Ilumya (tildrakizumab-asmn); Imfinzi (durvalumab); Increlex (mecasermin); Infasurf (calfactant); Infergen (interferon alfacon-1); Inflectra (infliximab-dyyb); Intron A (interferon alfa-2b); Iplex (mecasermin rinfabate); Iprivask (desirudin); Jeanatope (kit for iodinated 1-125 albumin); Jetrea (ocriplasmin); Jeuveau (prabotulinumtoxinA-xvfs); Kadcyla (ado-trastuzumab emtansine); Kalbitor (ecallantide); Kanjinti (trastuzumab-anns); Kanuma (sebelipase alfa); Kepivance (palifermin); Kevzara (sarilumab); Keytruda (pembrolizumab); Kineret (anakinra); Kinlytic (urokinase); Krystexxa (pegloticase); Lantus (insulin glargine); Lartruvo (olaratumab); Lemtrada (alemtuzumab); Leukine (sargramostim); Levemir (insulin detemir); Libtayo (cemiplimab-rwlc); Lucentis (ranibizumab); Lumizyme (alglucosidase alfa); Lumoxiti (moxetumomab pasudotox-tdfk); Macrotec (kit for the preparation of technetium Tc-99m albumin aggregated); Megatope (kit for iodinated 1-131 albumin); Menopur (menotropins); Mepsevii (vestronidase alfa-vjbk); Microlite (radiolabeled albumin technetium Tc-99m albumin colloid kit); Mircera (methoxy polyethylene glycol-epoetin beta); Mvasi (bevacizumab-awwb); Myalept (metreleptin); Mylotarg (gemtuzumab ozogamicin); Myobloc (rimabotulinumtoxinB); Myozyme (alglucosidase alfa); Myxredlin (insulin human); N/A (raxibacumab); Naglazyme (galsulfase); Natpara (parathyroid hormone); Neulasta (pegfilgrastim); Neulasta Onpro (pegfilgrastim); Neumega (oprelvekin); Neupogen (filgrastim); NeutroSpec (technetium 99m tc fanolesomab); Nivestym (filgrastim-aafi); Norditropin (somatropin); Novarel (chorionic gonadotropin); Novolin 70/30 (insulin isophane human and insulin human); Novolin N (insulin isophane human); Novolin R (insulin human); Novolog (insulin aspart); Novolog Mix 50/50 (insulin aspart protamine and insulin aspart); Novolog Mix 70/30 (insulin aspart protamine and insulin aspart); Nplate (romiplostim); Nucala (mepolizumab); Nulojix (belatacept); Nutropin (somatropin); Nutropin AQ (somatropin); Ocrevus (ocrelizumab); Omnitrope (somatropin); Oncaspar (pegaspargase); Ontak (denileukin diftitox); Ontruzant (trastuzumab-dttb); Opdivo (nivolumab); Orencia (abatacept); Orthoclone OKT3 (muromanab-CD3); Ovidrel (choriogonadotropin alfa); Oxervate (cenegermin-bkbj); Padcev (enfortumab vedotin-ejfv); Palynziq (pegvaliase-pqpz); Pancreaze (pancrelipase); Pegasys (peginterferon alfa-2a); Pegasys Copegus Combination Pack (peginterferon alfa-2a and ribavirin); Pegintron (peginterferon alfa-2b); Peglntron/Rebetol Combo Pack (peginterferon alfa-2b and ribavirin); Pergonal (menotropins); Perjeta (pertuzumab); Pertzye (pancrelipase); Plegridy (peginterferon beta-1a); Polivy (polatuzumab vedotin-piiq); Portrazza (necitumumab); Poteligeo (mogamulizumab-kpkc); Praluent (alirocumab); Praxbind (idarucizumab); Pregnyl (chorionic gonadotropin); Procrit (epoetin alfa); Proleukin (aldesleukin); Prolia (denosumab); ProstaScint (capromab pendetide); Pulmolite (kit for the preparation of technetium Tc-99m albumin aggregated); Pulmotech MAA (kit for the preparation of technetium Tc-99m albumin aggregated); Pulmozyme (domase alfa); Raptiva (efalizumab); Rebif (interferon beta-1a); Reblozyl (luspatercept-aamt); Regranex (becaplermin); Remicade (infliximab); Renflexis (infliximab-abda); Reopro (abciximab); Repatha (evolocumab); Repronex (menotropins); Retacrit (epoetin alfa-epbx); Retavase (reteplase); Revcovi (elapegademase-lvlr); Rituxan (rituximab); Rituxan Hycela (rituximab and hyaluronidase human); Roferon-A (interferon alfa-2a); Ruxience (rituximab-pvvr); Ryzodeg 70/30 (insulin degludec and insulin aspart); Saizen (somatropin); Santyl (collagenase); Sarclisa (isatuximab-irfc); Serostim (somatropin); Siliq (brodalumab); Simponi (golimumab); Simponi Aria (golimumab); Simulect (basiliximab); Skyrizi (risankizumab-rzaa); Soliqua 100/33 (insulin glargine and lixisenatide); Solirs (eculizumab); Somavert (pegvisomant); Stelara (ustekinumab); Strensiq (asfotas alfa); Sucraid (sacrosidase); Survanta (beractant); Sylvant (siltuximab); Synagis (palivizumab); Takhzyro (lanadelumab-flyo); Taltz (ixekizumab); Tanzeum (albiglutide); Tecentriq (atezolizumab); Tepezza (teprotumumab-trbw); Thyrogen (thyrotropin alfa); TNKase (tenecteplase); Toujeo (insulin glargine); Trasylol (aprotinin); Trazimera (trastuzumab-qyyp); Tremfya (guselkumab); Tresiba (insulin degludec); Trodelvy (sacituzumab govitecan-hziy); Trogarzo (ibalizumab-uiyk); Trulicity (dulaglutide); Truxima (rituximab-abbs); Tysabri (natalizumab); Udenyca (pegfilgrastim-cbqv); Ultomiris (ravulizumab-cwvz); Unituxin (dinutuximab); Vectibix (panitumumab); Verluma (nofetumomab); Vimizim (elosulfase alfa); Viokace (pancrelipase); Vitrase (hyaluronidase); Voraxaze (glucarpidase); VPRIV (velaglucerase alfa); Xeomin (incobotulinumtoxinA); Xgeva (denosumab); Xiaflex (collagenase clostridium histolyticum); Xigris (drotrecogin alfa); Xolair (omalizumab); Xultophy 100/3.6 (insulin degludec and liraglutide); Yervoy (ipilimumab); Zaltrap (Ziv-Aflibercept); Zarxio (filgrastim-sndz); Zenapax (daclizumab); Zenpep (pancrelipase); Zevalin (ibritumomab tiuxetan); Ziextenzo (pegfilgrastim-bmez); Zinbryta (daclizumab); Zinplava (beziotoxumab); Zirabev (bevacizumab-bvzr); Zomacton (somatropin); Zorbtive/Serostim (somatropin);

### Inhalation Anesthetics

Aliflurane; Chloroform; Cyclopropane; Desflurane (Suprane); Diethyl Ether; Enflurane (Ethrane); Ethyl Chloride; Ethylene; Halothane (Fluothane); Isoflurane (Forane, Isoflo); Isopropenyl vinyl ether; Methoxyflurane; methoxyflurane; Methoxypropane; Nitrous Oxide; Roflurane; Sevoflurane (Sevorane, Ultane, Sevoflo); Teflurane; Trichloroethylene; Vinyl Ether; Xenon

### Injectable Drugs

Ablavar (Gadofosveset Trisodium Injection); Abarelix Depot; Abobotulinumtoxin A Injection (Dysport); ABT-263; ABT-869; ABX-EFG; Accretropin (Somatropin Injection); Acetadote (Acetylcysteine Injection); Acetazolamide Injection (Acetazolamide Injection); Acetylcysteine Injection (Acetadote); Actemra (Tocilizumab Injection); Acthrel (Corticorelin Ovine Triflutate for Injection); Actummune; Activase; Acyclovir for Injection (Zovirax Injection); Adacel; Adalimumab; Adenoscan (Adenosine Injection); Adenosine Injection (Adenoscan); Adrenaclick; AdreView (lobenguane I 123 Injection for Intravenous Use); Afluria; Ak-Fluor (Fluorescein Injection); Aldurazyme (Laronidase); Alglucerase Injection (Ceredase); Alkeran Injection (Melphalan Hcl Injection); Allopurinol Sodium for Injection (Aloprim); Aloprim (Allopurinol Sodium for Injection); Alprostadil; Alsuma (Sumatriptan Injection); ALTU-238; Amino Acid Injections; Aminosyn; Apidra; Apremilast; Alprostadil Dual Chamber System for Injection (Caverject Impulse); AMG 009; AMG 076; AMG 102; AMG 108; AMG 114; AMG 162; AMG 220; AMG 221; AMG 222; AMG 223; AMG 317; AMG 379; AMG 386; AMG 403; AMG 477; AMG 479; AMG 517; AMG 531; AMG 557; AMG 623; AMG 655; AMG 706; AMG 714; AMG 745; AMG 785; AMG 811; AMG 827; AMG 837; AMG 853; AMG 951; Amiodarone HCl Injection (Amiodarone HCl Injection); Amobarbital Sodium Injection (Amytal Sodium); Amytal Sodium (Amobarbital Sodium Injection); Anakinra; Anti-Abeta; Anti-Beta7; Anti-Beta20; Anti-CD4; Anti-CD20; Anti-CD40; Anti-IFNalpha; Anti-IL13; Anti-OX40L; Anti-oxLDS; Anti-NGF; Anti-NRP1; Arixtra; Amphadase (Hyaluronidase Inj); Ammonul (Sodium Phenylacetate and Sodium Benzoate Injection); Anaprox; Anzemet Injection (Dolasetron Mesylate Injection); Apidra (Insulin Glulisine [rDNA origin] Inj); Apomab; Aranesp (darbepoetin alfa); Argatroban (Argatroban Injection); Arginine Hydrochloride Injection (R-Gene 10); Aristocort; Aristospan; Arsenic Trioxide Injection (Trisenox); Articane HCl and Epinephrine Injection (Septocaine); Arzerra (Ofatumumab Injection); Asclera (Polidocanol Injection); Ataluren; Ataluren-DMD; Atenolol Inj (Tenormin I.V. Injection); Atracurium Besylate Injection (Atracurium Besylate Injection); Avastin; Azactam Injection (Aztreonam Injection); Azithromycin (Zithromax Injection); Aztreonam Injection (Azactam Injection); Baclofen Injection (Lioresal Intrathecal); Bacteriostatic Water (Bacteriostatic Water for Injection); Baclofen Injection (Lioresal Intrathecal); Bal in Oil Ampules (Dimercarprol Injection); BayHepB; BayTet; Benadryl; Bendamustine Hydrochloride Injection (Treanda); Benztropine Mesylate Injection (Cogentin); Betamethasone Injectable Suspension (Celestone Soluspan); Bexxar; Bicillin C-R 900/300 (Penicillin G Benzathine and Penicillin G Procaine Injection); Blenoxane (Bleomycin Sulfate Injection); Bleomycin Sulfate Injection (Blenoxane); Boniva Injection (Ibandronate Sodium Injection); Botox Cosmetic (OnabotulinumtoxinA for Injection); BR3-FC; Bravelle (Urofollitropin Injection); Bretylium (Bretylium Tosylate Injection); Brevital Sodium (Methohexital Sodium for Injection); Brethine; Briobacept; BTT-1023; Bupivacaine HCl; Byetta; Ca-DTPA (Pentetate Calcium Trisodium Inj); Cabazitaxel Injection (Jevtana); Caffeine Alkaloid (Caffeine and Sodium Benzoate Injection); Calcijex Injection (Calcitrol); Calcitrol (Calcijex Injection); Calcium Chloride (Calcium Chloride Injection 10%); Calcium Disodium Versenate (Edetate Calcium Disodium Injection); Campath (Altemtuzumab); Camptosar Injection (Irinotecan Hydrochloride); Canakinumab Injection (Ilaris); Capastat Sulfate (Capreomycin for Injection); Capreomycin for Injection (Capastat Sulfate); Cardiolite (Prep kit for Technetium Tc99 Sestamibi for Injection); Carticel; Cathflo; Cefazolin and Dextrose for Injection (Cefazolin Injection); Cefepime Hydrochloride; Cefotaxime; Ceftriaxone; Cerezyme; Camitor Injection; Caverject; Celestone Soluspan; Celsior; Cerebyx (Fosphenytoin Sodium Injection); Ceredase (Alglucerase Injection); Ceretec (Technetium Tc99m Exametazime Injection); Certolizumab; CF-101; Chloramphenicol Sodium Succinate (Chloramphenicol Sodium Succinate Injection); Chloramphenicol Sodium Succinate Injection (Chloramphenicol Sodium Succinate); Cholestagel (Colesevelam HCL); Choriogonadotropin Alfa Injection (Ovidrel); Cimzia; Cisplatin (Cisplatin Injection); Clolar (Clofarabine Injection); Clomiphine Citrate; Clonidine Injection (Duraclon); Cogentin (Benztropine Mesylate Injection); Colistimethate Injection (Coly-Mycin M); Coly-Mycin M (Colistimethate Injection); Compath; Conivaptan Hcl Injection (Vaprisol); Conjugated Estrogens for Injection (Premarin Injection); Copaxone; Corticorelin Ovine Triflutate for Injection (Acthrel); Corvert (lbutilide Fumarate Injection); Cubicin (Daptomycin Injection); CF-101; Cyanokit (Hydroxocobalamin for Injection); Cytarabine Liposome Injection (DepoCyt); Cyanocobalamin; Cytovene (ganciclovir); D.H.E. 45; Dacetuzumab; Dacogen (Decitabine Injection); Dalteparin; Dantrium IV (Dantrolene Sodium for Injection); Dantrolene Sodium for Injection (Dantrium IV); Daptomycin Injection (Cubicin); Darbepoietin Alfa; DDAVP Injection (Desmopressin Acetate Injection); Decavax; Decitabine Injection (Dacogen); Dehydrated Alcohol (Dehydrated Alcohol Injection); Denosumab Injection (Prolia); Delatestryl; Delestrogen; Delteparin Sodium; Depacon (Valproate Sodium Injection); Depo Medrol (Methylprednisolone Acetate Injectable Suspension); DepoCyt (Cytarabine Liposome Injection); DepoDur (Morphine Sulfate XR Liposome Injection); Desmopressin Acetate Injection (DDAVP Injection); Depo-Estradiol; Depo-Provera 104 mg/ml; Depo-Provera 150 mg/ml; Depo-Testosterone; Dexrazoxane for Injection, Intravenous Infusion Only (Totect); Dextrose/Electrolytes; Dextrose and Sodium Chloride Inj (Dextrose 5% in 0.9% Sodium Chloride); Dextrose; Diazepam Injection (Diazepam Injection); Digoxin Injection (Lanoxin Injection); Dilaudid-HP (Hydromorphone Hydrochloride Injection); Dimercarprol Injection (Bal in Oil Ampules); Diphenhydramine Injection (Benadryl Injection); Dipyridamole Injection (Dipyridamole Injection); DMOAD; Docetaxel for Injection (Taxotere); Dolasetron Mesylate Injection (Anzemet Injection); Doribax (Doripenem for Injection); Doripenem for Injection (Doribax); Doxercalciferol Injection (Hectorol Injection); Doxil (Doxorubicin Hcl Liposome Injection); Doxorubicin Hcl Liposome Injection (Doxil); Duraclon (Clonidine Injection); Duramorph (Morphine Injection); Dysport (Abobotulinumtoxin A Injection); Ecallantide Injection (Kalbitor); EC-Naprosyn (naproxen); Edetate Calcium Disodium Injection (Calcium Disodium Versenate); Edex (Alprostadil for Injection); Engerix; Edrophonium Injection (Enlon); Eliglustat Tartate; Eloxatin (Oxaliplatin Injection); Emend Injection (Fosaprepitant Dimeglumine Injection); Enalaprilat Injection (Enalaprilat Injection); Enlon (Edrophonium Injection); Enoxaparin Sodium Injection (Lovenox); Eovist (Gadoxetate Disodium Injection); Enbrel (etanercept); Enoxaparin; Epicel; Epinepherine; Epipen; Epipen Jr.; Epratuzumab; Erbitux; Ertapenem Injection (Invanz); Erythropoieten; Essential Amino Acid Injection (Nephramine); Estradiol Cypionate; Estradiol Valerate; Etanercept; Exenatide Injection (Byetta); Evlotra; Fabrazyme (Adalsidase beta); Famotidine Injection; FDG (Fludeoxyglucose F 18 Injection); Feraheme (Ferumoxytol Injection); Feridex I.V. (Ferumoxides Injectable Solution); Fertinex; Ferumoxides Injectable Solution (Feridex I.V.); Ferumoxytol Injection (Feraheme); Flagyl Injection (Metronidazole Injection); Fluarix; Fludara (Fludarabine Phosphate); Fludeoxyglucose F 18 Injection (FDG); Fluorescein Injection (Ak-Fluor); Follistim AQ Cartridge (Follitropin Beta Injection); Follitropin Alfa Injection (Gonal-f RFF); Follitropin Beta Injection (Follistim AQ Cartridge); Folotyn (Pralatrexate Solution for Intravenous Injection); Fondaparinux; Forteo (Teriparatide (rDNA origin) Injection); Fostamatinib; Fosaprepitant Dimeglumine Injection (Emend Injection); Foscarnet Sodium Injection (Foscavir); Foscavir (Foscarnet Sodium Injection); Fosphenytoin Sodium Injection (Cerebyx); Fospropofol Disodium Injection (Lusedra); Fragmin; Fuzeon (enfuvirtide); GA101; Gadobenate Dimeglumine Injection (Multihance); Gadofosveset Trisodium Injection (Ablavar); Gadoteridol Injection Solution (ProHance); Gadoversetamide Injection (OptiMARK); Gadoxetate Disodium Injection (Eovist); Ganirelix (Ganirelix Acetate Injection); Gardasil; GC1008; GDFD; Gemtuzumab Ozogamicin for Injection (Mylotarg); Genotropin; Gentamicin Injection; GENZ-112638; Golimumab Injection (Simponi Injection); Gonal-f RFF (Follitropin Alfa Injection); Granisetron Hydrochloride (Kytril Injection); Gentamicin Sulfate; Glatiramer Acetate; Glucagen; Glucagon; HAE1; Haldol (Haloperidol Injection); Havrix; Hectorol Injection (Doxercalciferol Injection); Hedgehog Pathway Inhibitor; Heparin; Herceptin; hG-CSF; Humalog; Human Growth Hormone; Humatrope; HuMax; Humegon; Humira; Humulin; Ibandronate Sodium Injection (Boniva Injection); Ibuprofen Lysine Injection (NeoProfen); Ibutilide Fumarate Injection (Corvert); Idamycin PFS (Idarubicin Hydrochloride Injection); Idarubicin Hydrochloride Injection (Idamycin PFS); Ilaris (Canakinumab Injection); Imipenem and Cilastatin for Injection (Primaxin I.V.); Imitrex; Incobotulinumtoxin A for Injection (Xeomin); Increlex (Mecasermin [rDNA origin] Injection); Indocin IV (Indomethacin Inj); Indomethacin Inj (Indocin IV); Infanrix; Innohep; Insulin; Insulin Aspart [rDNA origin] Inj (NovoLog); Insulin Glargine [rDNA origin] Injection (Lantus); Insulin Glulisine [rDNA origin] Inj (Apidra); Interferon alfa-2b, Recombinant for Injection (Intron A); Intron A (Interferon alfa-2b, Recombinant for Injection); Invanz (Ertapenem Injection); Invega Sustenna (Paliperidone Palmitate Extended-Release Injectable Suspension); Invirase (saquinavir mesylate); lobenguane I 123 Injection for Intravenous Use (AdreView); lopromide Injection (Ultravist); loversol Injection (Optiray Injection); Iplex (Mecasermin Rinfabate [rDNA origin] Injection); lprivask; Irinotecan Hydrochloride (Camptosar Injection); Iron Sucrose Injection (Venofer); Istodax (Romidepsin for Injection); Itraconazole Injection (Sporanox Injection); Jevtana (Cabazitaxel Injection); Jonexa; Kalbitor (Ecallantide Injection); KCL in D5NS (Potassium Chloride in 5% Dextrose and Sodium Chloride Injection); KCL in D5W; KCL in NS; Kenalog 10 Injection (Triamcinolone Acetonide Injectable Suspension); Kepivance (Palifermin); Keppra Injection (Levetiracetam); Keratinocyte; KFG; Kinase Inhibitor; Kineret (Anakinra); Kinlytic (Urokinase Injection); Kinrix; Klonopin (clonazepam); Kytril Injection (Granisetron Hydrochloride); lacosamide Tablet and Injection (Vimpat); Lactated Ringers; Lanoxin Injection (Digoxin Injection); Lansoprazole for Injection (Prevacid I.V.); Lantus; Leucovorin Calcium (Leucovorin Calcium Injection); Lente (L); Leptin; Levemir; Leukine Sargramostim; Leuprolide Acetate; Levothyroxine;
Levetiracetam (Keppra Injection); Lovenox; Levocarnitine Injection (Camitor Injection); Lexiscan (Regadenoson Injection); Lioresal Intrathecal (Baclofen Injection); Liraglutide [rDNA] Injection (Victoza); Lovenox (Enoxaparin Sodium Injection); Lucentis (Ranibizumab Injection); Lumizyme; Lupron (Leuprolide Acetate Injection); Lusedra (Fospropofol Disodium Injection); Maci; Magnesium Sulfate (Magnesium Sulfate Injection); Mannitol Injection (Mannitol IV); Marcaine (Bupivacaine Hydrochloride and Epinephrine Injection); Maxipime (Cefepime Hydrochloride for Injection); MDP Multidose Kit of Technetium Injection (Technetium Tc99m Medronate Injection); Mecasermin [rDNA origin] Injection (Increlex); Mecasermin Rinfabate [rDNA origin] Injection (Iplex); Melphalan Hcl Injection (Alkeran Injection); Methotrexate; Menactra; Menopur (Menotropins Injection); Menotropins for Injection (Repronex); Methohexital Sodium for Injection (Brevital Sodium); Methyldopate Hydrochloride Injection, Solution (Methyldopate Hcl); Methylene Blue (Methylene Blue Injection); Methylprednisolone Acetate Injectable Suspension (Depo Medrol); MetMab; Metoclopramide Injection (Reglan Injection); Metrodin (Urofollitropin for Injection); Metronidazole Injection (Flagyl Injection); Miacalcin; Midazolam (Midazolam Injection); Mimpara (Cinacalet); Minocin Injection (Minocycline Inj); Minocycline Inj (Minocin Injection); Mipomersen; Mitoxantrone for Injection Concentrate (Novantrone); Morphine Injection (Duramorph); Morphine Sulfate XR Liposome Injection (DepoDur); Morrhuate Sodium (Morrhuate Sodium Injection); Motesanib; Mozobil (Plerixafor Injection); Multihance (Gadobenate Dimeglumine Injection); Multiple Electrolytes and Dextrose Injection; Multiple Electrolytes Injection; Mylotarg (Gemtuzumab Ozogamicin for Injection); Myozyme (Alglucosidase alfa); Nafcillin Injection (Nafcillin Sodium); Nafcillin Sodium (Nafcillin Injection); Naltrexone XR Inj (Vivitrol); Naprosyn (naproxen); NeoProfen (lbuprofen Lysine Injection); Nandrol Decanoate; Neostigmine Methylsulfate (Neostigmine Methylsulfate Injection); NEO-GAA; NeoTect (Technetium Tc 99m Depreotide Injection); Nephramine (Essential Amino Acid Injection); Neulasta (pegfilgrastim); Neupogen (Filgrastim); Novolin; Novolog; NeoRecormon; Neutrexin (Trimetrexate Glucuronate Inj); NPH (N); Nexterone (Amiodarone HCl Injection); Norditropin (Somatropin Injection); Normal Saline (Sodium Chloride Injection); Novantrone (Mitoxantrone for Injection Concentrate); Novolin 70/30 Innolet (70% NPH, Human Insulin Isophane Suspension and 30% Regular, Human Insulin Injection); NovoLog (Insulin Aspart [rDNA origin] Inj); Nplate (romiplostim); Nutropin (Somatropin (rDNA origin) for Inj); Nutropin AQ; Nutropin Depot (Somatropin (rDNA origin) for Inj); Octreotide Acetate Injection (Sandostatin LAR); Ocrelizumab; Ofatumumab Injection (Arzerra); Olanzapine Extended Release Injectable Suspension (Zyprexa Relprevv); Omnitarg; Omnitrope (Somatropin [rDNA origin] Injection); Ondansetron Hydrochloride Injection (Zofran Injection); OptiMARK (Gadoversetamide Injection); Optiray Injection (loversol Injection); Orencia; Osmitrol Injection in Aviva (Mannitol Injection in Aviva Plastic Vessel); Osmitrol Injection in Viaflex (Mannitol Injection in Viaflex Plastic Vessel); Osteoprotegrin; Ovidrel (Choriogonadotropin Alfa Injection); Oxacillin (Oxacillin for Injection); Oxaliplatin Injection (Eloxatin); Oxytocin Injection (Pitocin); Paliperidone Palmitate Extended-Release Injectable Suspension (Invega Sustenna); Pamidronate Disodium Injection (Pamidronate Disodium Injection); Panitumumab Injection for Intravenous Use (Vectibix); Papaverine Hydrochloride Injection (Papaverine Injection); Papaverine Injection (Papaverine Hydrochloride Injection); Parathyroid Hormone; Paricalcitol Injection Fliptop Vial (Zemplar Injection); PARP Inhibitor; Pediarix; PEGlntron; Peginterferon; Pegfilgrastim; Penicillin G Benzathine and Penicillin G Procaine; Pentetate Calcium Trisodium Inj (Ca-DTPA); Pentetate Zinc Trisodium Injection (Zn-DTPA); Pepcid Injection (Famotidine Injection); Pergonal; Pertuzumab; Phentolamine Mesylate (Phentolamine Mesylate for Injection); Physostigmine Salicylate (Physostigmine Salicylate (injection)); Physostigmine Salicylate (injection) (Physostigmine Salicylate); Piperacillin and Tazobactam Injection (Zosyn); Pitocin (Oxytocin Injection); Plasma-Lyte 148 (Multiple Electrolytes Inj); Plasma-Lyte 56 and Dextrose (Multiple Electrolytes and Dextrose Injection in Viaflex Plastic Vessel); PlasmaLyte; Plerixafor Injection (Mozobil); Polidocanol Injection (Asclera); Potassium Chloride; Pralatrexate Solution for Intravenous Injection (Folotyn); Pramlintide Acetate Injection (Symlin); Premarin Injection (Conjugated Estrogens for Injection); Prep kit for Technetium Tc99 Sestamibi for Injection (Cardiolite); Prevacid I.V. (Lansoprazole for Injection); Primaxin I.V. (Imipenem and Cilastatin for Injection); Prochymal; Procrit; Progesterone; ProHance (Gadoteridol Injection Solution); Prolia (Denosumab Injection); Promethazine HCl Injection (Promethazine Hydrochloride Injection); Propranolol Hydrochloride Injection (Propranolol Hydrochloride Injection); Quinidine Gluconate Injection (Quinidine Injection); Quinidine Injection (Quinidine Gluconate Injection); R-Gene 10 (Arginine Hydrochloride Injection); Ranibizumab Injection (Lucentis); Ranitidine Hydrochloride Injection (Zantac Injection); Raptiva; Reclast (Zoledronic Acid Injection); Recombivarix HB; Regadenoson Injection (Lexiscan); Reglan Injection (Metoclopramide Injection); Remicade; Renagel; Renvela (Sevelamer Carbonate); Repronex (Menotropins for Injection); Retrovir IV (Zidovudine Injection); rhApo2L/TRAIL; Ringers and 5% Dextrose Injection (Ringers in Dextrose); Ringer's Injection (Ringers Injection); Rituxan; Rituximab; Rocephin (ceftriaxone); Rocuronium Bromide Injection (Zemuron); Roferon-A (interferon alfa-2a); Romazicon (flumazenil); Romidepsin for Injection (Istodax); Saizen (Somatropin Injection); Sandostatin LAR (Octreotide Acetate Injection); Scerostin Ab; Sensipar (cinacalcet); Sensorcaine (Bupivacaine HCl Injections); Septocaine (Articane HCl and Epinephrine Injection); Serostim LQ (Somatropin (rDNA origin) Injection); Simponi Injection (Golimumab Injection); Sodium Acetate (Sodium Acetate Injection); Sodium Bicarbonate (Sodium Bicarbonate 5% Injection); Sodium Lactate (Sodium Lactate Injection in AVIVA); Sodium Phenylacetate and Sodium Benzoate Injection (Ammonul); Somatropin (rDNA origin) for Inj (Nutropin); Sporanox Injection (Itraconazole Injection); Stelara Injection (Ustekinumab); Stemgen; Sufenta (Sufentanil Citrate Injection); Sufentanil Citrate Injection (Sufenta); Sumavel; Sumatriptan Injection (Alsuma); Symlin; Symlin Pen; Systemic Hedgehog Antagonist; Synvisc-One (Hylan G-F 20 Single Intra-articular Injection); Tarceva; Taxotere (Docetaxel for Injection); Technetium Tc 99m; Telavancin for Injection (Vibativ); Temsirolimus Injection (Torisel); Tenormin I.V. Injection (Atenolol Inj); Teriparatide (rDNA origin) Injection (Forteo); Testosterone Cypionate; Testosterone Enanthate; Testosterone Propionate; Tev-Tropin (Somatropin, rDNA Origin, for Injection); tgAAC94; Thallous Chloride; Theophylline; Thiotepa (Thiotepa Injection); Thymoglobulin (Anti-Thymocyte Globulin (Rabbit); Thyrogen (Thyrotropin Alfa for Injection); Ticarcillin Disodium and Clavulanate Potassium Galaxy (Timentin Injection); Tigan Injection (Trimethobenzamide Hydrochloride Injectable); Timentin Injection (Ticarcillin Disodium and Clavulanate Potassium Galaxy); TNKase; Tobramycin Injection (Tobramycin Injection); Tocilizumab Injection (Actemra); Torisel (Temsirolimus Injection); Totect (Dexrazoxane for Injection, Intravenous Infusion Only); Trastuzumab-DM1; Travasol (Amino Acids (Injection)); Treanda (Bendamustine Hydrochloride Injection); Trelstar (Triptorelin Pamoate for Injectable Suspension); Triamcinolone Acetonide; Triamcinolone Diacetate; Triamcinolone Hexacetonide Injectable Suspension (Aristospan Injection 20 mg); Triesence (Triamcinolone Acetonide Injectable Suspension); Trimethobenzamide Hydrochloride Injectable (Tigan Injection); Trimetrexate Glucuronate Inj (Neutrexin); Triptorelin Pamoate for Injectable Suspension (Trelstar); Twinject; Trivaris (Triamcinolone Acetonide Injectable Suspension); Trisenox (Arsenic Trioxide Injection); Twinrix; Typhoid Vi; Ultravist (lopromide Injection); Urofollitropin for Injection (Metrodin); Urokinase Injection (Kinlytic); Ustekinumab (Stelara Injection); Ultralente (U); Valium (diazepam); Valproate Sodium Injection (Depacon); Valtropin (Somatropin Injection); Vancomycin Hydrochloride (Vancomycin Hydrochloride Injection); Vancomycin Hydrochloride Injection (Vancomycin Hydrochloride); Vaprisol (Conivaptan Hcl Injection); VAQTA; Vasovist (Gadofosveset Trisodium Injection for Intravenous Use); Vectibix (Panitumumab Injection for Intravenous Use); Venofer (Iron Sucrose Injection); Verteporfin Inj (Visudyne); Vibativ (Telavancin for Injection); Victoza (Liraglutide [rDNA] Injection); Vimpat (lacosamide Tablet and Injection); Vinblastine Sulfate (Vinblastine Sulfate Injection); Vincasar PFS (Vincristine Sulfate Injection); Victoza; Vincristine Sulfate (Vincristine Sulfate Injection); Visudyne (Verteporfin Inj); Vitamin B-12; Vivitrol (Naltrexone XR Inj); Voluven (Hydroxyethyl Starch in Sodium Chloride Injection); Xeloda; Xenical (orlistat); Xeomin (Incobotulinumtoxin A for Injection); Xolair; Zantac Injection (Ranitidine Hydrochloride Injection); Zemplar Injection (Paricalcitol Injection Fliptop Vial); Zemuron (Rocuronium Bromide Injection); Zenapax (daclizumab); Zevalin; Zidovudine Injection (Retrovir IV); Zithromax Injection (Azithromycin); Zn-DTPA (Pentetate Zinc Trisodium Injection); Zofran Injection (Ondansetron Hydrochloride Injection); Zingo; Zoledronic Acid for Inj (Zometa); Zoledronic Acid Injection (Reclast); Zometa (Zoledronic Acid for Inj); Zosyn (Piperacillin and Tazobactam Injection); Zyprexa Relprevv (Olanzapine Extended Release Injectable Suspension)

### Liquid Drugs (Non-Injectable)

Abilify; AccuNeb (Albuterol Sulfate Inhalation Solution); Actidose Aqua (Activated Charcoal Suspension); Activated Charcoal Suspension (Actidose Aqua); Advair; Agenerase Oral Solution (Amprenavir Oral Solution); Akten (Lidocaine Hydrochloride Ophthalmic Gel); Alamast (Pemirolast Potassium Ophthalmic Solution); Albumin (Human) 5% Solution (Buminate 5%); Albuterol Sulfate Inhalation Solution; Alinia; Alocril; Alphagan; Alrex; Alvesco; Amprenavir Oral Solution; Analpram-HC; Arformoterol Tartrate Inhalation Solution (Brovana); Aristospan Injection 20 mg (Triamcinolone Hexacetonide Injectable Suspension); Asacol; Asmanex; Astepro; Astepro (Azelastine Hydrochloride Nasal Spray); Atrovent Nasal Spray (Ipratropium Bromide Nasal Spray); Atrovent Nasal Spray 0.06; Augmentin ES-600; Azasite (Azithromycin Ophthalmic Solution); Azelaic Acid (Finacea Gel); Azelastine Hydrochloride Nasal Spray (Astepro); Azelex (Azelaic Acid Cream); Azopt (Brinzolamide Ophthalmic Suspension); Bacteriostatic Saline; Balanced Salt; Bepotastine; Bactroban Nasal; Bactroban; Beclovent; Benzac W; Betimol; Betoptic S; Bepreve; Bimatoprost Ophthalmic Solution; Bleph 10 (Sulfacetamide Sodium Ophthalmic Solution 10%); Brinzolamide Ophthalmic Suspension (Azopt); Bromfenac Ophthalmic Solution (Xibrom); Bromhist; Brovana (Arformoterol Tartrate Inhalation Solution); Budesonide Inhalation Suspension (Pulmicort Respules); Cambia (Diclofenac Potassium for Oral Solution); Capex; Carac; Carboxine-PSE; Carnitor; Cayston (Aztreonam for Inhalation Solution); Cellcept; Centany; Cerumenex; Ciloxan Ophthalmic Solution (Ciprofloxacin HCL Ophthalmic Solution); Ciprodex; Ciprofloxacin HCL Ophthalmic Solution (Ciloxan Ophthalmic Solution); Clemastine Fumarate Syrup (Clemastine Fumarate Syrup); CoLyte (PEG Electrolytes Solution); Combiven; Comtan; Condylox; Cordran; Cortisporin Ophthalmic Suspension; Cortisporin Otic Suspension; Cromolyn Sodium Inhalation Solution (Intal Nebulizer Solution); Cromolyn Sodium Ophthalmic Solution (Opticrom); Crystalline Amino Acid Solution with Electrolytes (Aminosyn Electrolytes); Cutivate; Cuvposa (Glycopyrrolate Oral Solution); Cyanocobalamin (CaloMist Nasal Spray); Cyclosporine Oral Solution (Gengraf Oral Solution); Cyclogyl; Cysview (Hexaminolevulinate Hydrochloride Intravesical Solution); DermOtic Oil (Fluocinolone Acetonide Oil Ear Drops); Desmopressin Acetate Nasal Spray; DDAVP; Derma-Smoothe/FS;
Dexamethasone Intensol; Dianeal Low Calcium; Dianeal PD; Diclofenac Potassium for Oral Solution (Cambia); Didanosine Pediatric Powder for Oral Solution (Videx); Differin; Dilantin 125 (Phenytoin Oral Suspension); Ditropan; Dorzolamide Hydrochloride Ophthalmic Solution (Trusopt); Dorzolamide Hydrochloride-Timolol Maleate Ophthalmic Solution (Cosopt); Dovonex Scalp (Calcipotriene Solution); Doxycycline Calcium Oral Suspension (Vibramycin Oral); Efudex; Elaprase (Idursulfase Solution); Elestat (Epinastine HCl Ophthalmic Solution); Elocon; Epinastine HCl Ophthalmic Solution (Elestat); Epivir HBV; Epogen (Epoetin alfa); Erythromycin Topical Solution 1.5% (Staticin); Ethiodol (Ethiodized Oil); Ethosuximide Oral Solution (Zarontin Oral Solution); Eurax; Extraneal (Icodextrin Peritoneal Dialysis Solution); Felbatol; Feridex I.V. (Ferumoxides Injectable Solution); Flovent; Floxin Otic (Ofloxacin Otic Solution); Flo-Pred (Prednisolone Acetate Oral Suspension); Fluoroplex; Flunisolide Nasal Solution (Flunisolide Nasal Spray 0.025%); Fluorometholone Ophthalmic Suspension (FML); Flurbiprofen Sodium Ophthalmic Solution (Ocufen); FML; Foradil; Formoterol Fumarate Inhalation Solution (Perforomist); Fosamax; Furadantin (Nitrofurantoin Oral Suspension); Furoxone; Gammagard Liquid (Immune Globulin Intravenous (Human) 10%); Gantrisin (Acetyl Sulfisoxazole Pediatric Suspension); Gatifloxacin Ophthalmic Solution (Zymar); Gengraf Oral Solution (Cyclosporine Oral Solution); Glycopyrrolate Oral Solution (Cuvposa); Halcinonide Topical Solution (Halog Solution); Halog Solution (Halcinonide Topical Solution); HEP-LOCK U/P (Preservative-Free Heparin Lock Flush Solution); Heparin Lock Flush Solution (Hepflush 10); Hexaminolevulinate Hydrochloride Intravesical Solution (Cysview); Hydrocodone Bitartrate and Acetaminophen Oral Solution (Lortab Elixir); Hydroquinone 3% Topical Solution (Melquin-3 Topical Solution); IAP Antagonist; Isopto; Ipratropium Bromide Nasal Spray (Atrovent Nasal Spray); Itraconazole Oral Solution (Sporanox Oral Solution); Ketorolac Tromethamine Ophthalmic Solution (Acular LS); Kaletra; Lanoxin; Lexiva; Leuprolide Acetate for Depot Suspension (Lupron Depot 11.25 mg); Levobetaxolol Hydrochloride Ophthalmic Suspension (Betaxon); Levocamitine Tablets, Oral Solution, Sugar-Free (Camitor); Levofloxacin Ophthalmic Solution 0.5% (Quixin); Lidocaine HCl Sterile Solution (Xylocaine MPF Sterile Solution); Lok Pak (Heparin Lock Flush Solution); Lorazepam Intensol; Lortab Elixir (Hydrocodone Bitartrate and Acetaminophen Oral Solution); Lotemax (Loteprednol Etabonate Ophthalmic Suspension); Loteprednol Etabonate Ophthalmic Suspension (Alrex); Low Calcium Peritoneal Dialysis Solutions (Dianeal Low Calcium); Lumigan (Bimatoprost Ophthalmic Solution 0.03% for Glaucoma); Lupron Depot 11.25 mg (Leuprolide Acetate for Depot Suspension); Megestrol Acetate Oral Suspension (Megestrol Acetate Oral Suspension); MEK Inhibitor; Mepron; Mesnex; Mestinon; Mesalamine Rectal Suspension Enema (Rowasa); Melquin-3 Topical Solution (Hydroquinone 3% Topical Solution); MetMab; Methyldopate Hcl (Methyldopate Hydrochloride Injection, Solution); Methylin Oral Solution (Methylphenidate HCl Oral Solution 5 mg/5 mL and 10 mg/5 mL); Methylprednisolone Acetate Injectable Suspension (Depo Medrol); Methylphenidate HCl Oral Solution 5 mg/5 mL and 10 mg/5 mL (Methylin Oral Solution); Methylprednisolone sodium succinate (Solu Medrol); Metipranolol Ophthalmic Solution (Optipranolol); Migranal; Miochol-E (Acetylcholine Chloride Intraocular Solution); Micro-K for Liquid Suspension (Potassium Chloride Extended Release Formulation for Liquid Suspension); Minocin (Minocycline Hydrochloride Oral Suspension); Nasacort; Neomycin and Polymyxin B Sulfates and Hydrocortisone; Nepafenac Ophthalmic Suspension (Nevanac); Nevanac (Nepafenac Ophthalmic Suspension); Nitrofurantoin Oral Suspension (Furadantin); Noxafil (Posaconazole Oral Suspension); Nystatin (oral) (Nystatin Oral Suspension); Nystatin Oral Suspension (Nystatin (oral)); Ocufen (Flurbiprofen Sodium Ophthalmic Solution); Ofloxacin Ophthalmic Solution (Ofloxacin Ophthalmic Solution); Ofloxacin Otic Solution (Floxin Otic); Olopatadine Hydrochloride Ophthalmic Solution (Pataday); Opticrom (Cromolyn Sodium Ophthalmic Solution); Optipranolol (Metipranolol Ophthalmic Solution); Patanol; Pediapred; PerioGard; Phenytoin Oral Suspension (Dilantin 125); Phisohex; Posaconazole Oral Suspension (Noxafil); Potassium Chloride Extended Release Formulation for Liquid Suspension (Micro-K for Liquid Suspension); Pataday (Olopatadine Hydrochloride Ophthalmic Solution); Patanase Nasal Spray (Olopatadine Hydrochloride Nasal Spray); PEG Electrolytes Solution (CoLyte); Pemirolast Potassium Ophthalmic Solution (Alamast); Penlac (Ciclopirox Topical Solution); PENNSAID (Diclofenac Sodium Topical Solution); Perforomist (Formoterol Fumarate Inhalation Solution); Peritoneal Dialysis Solution; Phenylephrine Hydrochloride Ophthalmic Solution (Neo-Synephrine); Phospholine Iodide (Echothiophate Iodide for Ophthalmic Solution); Podofilox (Podofilox Topical Solution); Pred Forte (Prednisolone Acetate Ophthalmic Suspension); Pralatrexate Solution for Intravenous Injection (Folotyn); Pred Mild; Prednisone Intensol; Prednisolone Acetate Ophthalmic Suspension (Pred Forte); Prevacid; PrismaSol Solution (Sterile Hemofiltration Hemodiafiltration Solution); ProAir; Proglycem; ProHance (Gadoteridol Injection Solution); Proparacaine Hydrochloride Ophthalmic Solution (Alcaine); Propine; Pulmicort; Pulmozyme; Quixin (Levofloxacin Ophthalmic Solution 0.5%); QVAR; Rapamune; Rebetol; Relacon-HC; Rotarix (Rotavirus Vaccine, Live, Oral Suspension); Rotavirus Vaccine, Live, Oral Suspension (Rotarix); Rowasa (Mesalamine Rectal Suspension Enema); Sabril (Vigabatrin Oral Solution); Sacrosidase Oral Solution (Sucraid); Sandimmune; Sepra; Serevent Diskus; Solu Cortef (Hydrocortisone Sodium Succinate); Solu Medrol (Methylprednisolone sodium succinate); Spiriva; Sporanox Oral Solution (Itraconazole Oral Solution); Staticin (Erythromycin Topical Solution 1.5%); Stalevo; Starlix; Sterile Hemofiltration Hemodiafiltration Solution (PrismaSol Solution); Stimate; Sucralfate (Carafate Suspension); Sulfacetamide Sodium Ophthalmic Solution 10% (Bleph 10); Synarel Nasal Solution (Nafarelin Acetate Nasal Solution for Endometriosis); Taclonex Scalp (Calcipotriene and Betamethasone Dipropionate Topical Suspension); Tamiflu; Tobi; TobraDex; Tobradex ST (Tobramycin/Dexamethasone Ophthalmic Suspension 0.3%/0.05%); Tobramycin/Dexamethasone Ophthalmic Suspension 0.3%/0.05% (Tobradex ST); Timolol; Timoptic; Travatan Z; Treprostinil Inhalation Solution (Tyvaso); Trusopt (Dorzolamide Hydrochloride Ophthalmic Solution); Tyvaso (Treprostinil Inhalation Solution); Ventolin; Vfend; Vibramycin Oral (Doxycycline Calcium Oral Suspension); Videx (Didanosine Pediatric Powder for Oral Solution); Vigabatrin Oral Solution (Sabril); Viokase; Viracept; Viramune; Vitamin K1 (Fluid Colloidal Solution of Vitamin K1); Voltaren Ophthalmic (Diclofenac Sodium Ophthalmic Solution); Zarontin Oral Solution (Ethosuximide Oral Solution); Ziagen; Zyvox; Zymar (Gatifloxacin Ophthalmic Solution); Zymaxid (Gatifloxacin Ophthalmic Solution)

### Drug Classes

5-alpha-reductase inhibitors; 5-aminosalicylates; 5HT3 receptor antagonists; adamantane antivirals; adrenal cortical steroids; adrenal corticosteroid inhibitors; adrenergic bronchodilators; agents for hypertensive emergencies; agents for pulmonary hypertension; aldosterone receptor antagonists; alkylating agents; alpha-adrenoreceptor antagonists; alpha-glucosidase inhibitors; alternative medicines; amebicides; aminoglycosides; aminopenicillins; aminosalicylates; amylin analogs; Analgesic Combinations; Analgesics; androgens and anabolic steroids; angiotensin converting enzyme inhibitors; angiotensin II inhibitors; anorectal preparations; anorexiants; antacids; anthelmintics; anti-angiogenic ophthalmic agents; anti-CTLA-4 monoclonal antibodies; anti-infectives; antiadrenergic agents, centrally acting; antiadrenergic agents, peripherally acting; antiandrogens; antianginal agents; antiarrhythmic agents; antiasthmatic combinations; antibiotics/antineoplastics; anticholinergic antiemetics; anticholinergic antiparkinson agents; anticholinergic bronchodilators; anticholinergic chronotropic agents; anticholinergics/antispasmodics; anticoagulants; anticonvulsants; antidepressants; antidiabetic agents; antidiabetic combinations; antidiarrheals; antidiuretic hormones; antidotes; antiemetic/antivertigo agents; antifungals; antigonadotropic agents; antigout agents; antihistamines; antihyperlipidemic agents; antihyperlipidemic combinations; antihypertensive combinations; antihyperuricemic agents; antimalarial agents; antimalarial combinations; antimalarial quinolines; antimetabolites; antimigraine agents; antineoplastic detoxifying agents; antineoplastic interferons; antineoplastic monoclonal antibodies; antineoplastics; antiparkinson agents; antiplatelet agents; antipseudomonal penicillins; antipsoriatics; antipsychotics; antirheumatics; antiseptic and germicides; antithyroid agents; antitoxins and antivenins; antituberculosis agents; antituberculosis combinations; antitussives; antiviral agents; antiviral combinations; antiviral interferons; anxiolytics, sedatives, and hypnotics; aromatase inhibitors; atypical antipsychotics; azole antifungals; bacterial vaccines; barbiturate anticonvulsants; barbiturates; BCR-ABL tyrosine kinase inhibitors; benzodiazepine anticonvulsants; benzodiazepines; beta-adrenergic blocking agents; beta-lactamase inhibitors; bile acid sequestrants; biologicals; bisphosphonates; bone resorption inhibitors; bronchodilator combinations; bronchodilators; calcitonin; calcium channel blocking agents; carbamate anticonvulsants; carbapenems; carbonic anhydrase inhibitor anticonvulsants; carbonic anhydrase inhibitors; cardiac stressing agents; cardioselective beta blockers; cardiovascular agents; catecholamines; CD20 monoclonal antibodies; CD33 monoclonal antibodies; CD52 monoclonal antibodies; central nervous system agents; cephalosporins; cerumenolytics; chelating agents; chemokine receptor antagonist; chloride channel activators; cholesterol absorption inhibitors; cholinergic agonists; cholinergic muscle stimulants; cholinesterase inhibitors; CNS stimulants; coagulation modifiers; colony stimulating factors; contraceptives; corticotropin; coumarins and indandiones; cox-2 inhibitors; decongestants; dermatological agents; diagnostic radiopharmaceuticals; dibenzazepine anticonvulsants; digestive enzymes; dipeptidyl peptidase 4 inhibitors; diuretics; dopaminergic antiparkinsonism agents; drugs used in alcohol dependence; echinocandins; EGFR inhibitors; estrogen receptor antagonists; estrogens; expectorants; factor Xa inhibitors; fatty acid derivative anticonvulsants; fibric acid derivatives; first generation cephalosporins; fourth generation cephalosporins; functional bowel disorder agents; gallstone solubilizing agents; gamma-aminobutyric acid analogs; gamma-aminobutyric acid reuptake inhibitors; gamma-aminobutyric acid transaminase inhibitors; gastrointestinal agents; general anesthetics; genitourinary tract agents; GI stimulants; glucocorticoids; glucose elevating agents; glycopeptide antibiotics; glycoprotein platelet inhibitors; glycylcyclines; gonadotropin releasing hormones; gonadotropin-releasing hormone antagonists; gonadotropins; group I antiarrhythmics; group II antiarrhythmics; group Ill antiarrhythmics; group IV antiarrhythmics; group V antiarrhythmics; growth hormone receptor blockers; growth hormones; H. pylori eradication agents; H2 antagonists; hematopoietic stem cell mobilizer; heparin antagonists; heparins; HER2 inhibitors; herbal products; histone deacetylase inhibitors; hormone replacement therapy; hormones; hormones/antineoplastics; hydantoin anticonvulsants; illicit (street) drugs; immune globulins; immunologic agents; immunosuppressive agents; impotence agents; in vivo diagnostic biologicals; incretin mimetics; inhaled anti-infectives; inhaled corticosteroids; inotropic agents; insulin; insulin-like growth factor; integrase strand transfer inhibitor; interferons; intravenous nutritional products; iodinated contrast media; ionic iodinated contrast media; iron
products; ketolides; laxatives; leprostatics; leukotriene modifiers; lincomycin derivatives; lipoglycopeptides; local injectable anesthetics; loop diuretics; lung surfactants; lymphatic staining agents; lysosomal enzymes; macrolide derivatives; macrolides; magnetic resonance imaging contrast media; mast cell stabilizers; medical gas; meglitinides; metabolic agents; methylxanthines; mineralocorticoids; minerals and electrolytes; miscellaneous agents; miscellaneous analgesics; miscellaneous antibiotics; miscellaneous anticonvulsants; miscellaneous antidepressants; miscellaneous antidiabetic agents; miscellaneous antiemetics; miscellaneous antifungals; miscellaneous antihyperlipidemic agents; miscellaneous antimalarials; miscellaneous antineoplastics; miscellaneous antiparkinson agents; miscellaneous antipsychotic agents; miscellaneous antituberculosis agents; miscellaneous antivirals; miscellaneous anxiolytics, sedatives and hypnotics; miscellaneous biologicals; miscellaneous bone resorption inhibitors; miscellaneous cardiovascular agents; miscellaneous central nervous system agents; miscellaneous coagulation modifiers; miscellaneous diuretics; miscellaneous genitourinary tract agents; miscellaneous GI agents; miscellaneous hormones; miscellaneous metabolic agents; miscellaneous ophthalmic agents; miscellaneous otic agents; miscellaneous respiratory agents; miscellaneous sex hormones; miscellaneous topical agents; miscellaneous uncategorized agents; miscellaneous vaginal agents; mitotic inhibitors; monoamine oxidase inhibitors; monoclonal antibodies; mouth and throat products; mTOR inhibitors; mTOR kinase inhibitors; mucolytics; multikinase inhibitors; muscle relaxants; mydriatics; narcotic analgesic combinations; narcotic analgesics; nasal anti-infectives; nasal antihistamines and decongestants; nasal lubricants and irrigations; nasal preparations; nasal steroids; natural penicillins; neuraminidase inhibitors; neuromuscular blocking agents; next generation cephalosporins; nicotinic acid derivatives; nitrates; NNRTIs; non-cardioselective beta blockers; non-iodinated contrast media; non-ionic iodinated contrast media; non-sulfonylureas; nonsteroidal anti-inflammatory agents; norepinephrine reuptake inhibitors; norepinephrine-dopamine reuptake inhibitors; nucleoside reverse transcriptase inhibitors (NRTIs); nutraceutical products; nutritional products; ophthalmic anesthetics; ophthalmic anti-infectives; ophthalmic anti-inflammatory agents; ophthalmic antihistamines and decongestants; ophthalmic diagnostic agents; ophthalmic glaucoma agents; ophthalmic lubricants and irrigations; ophthalmic preparations; ophthalmic steroids; ophthalmic steroids with anti-infectives; ophthalmic surgical agents; oral nutritional supplements; otic anesthetics; otic anti-infectives; otic preparations; otic steroids; otic steroids with anti-infectives; oxazolidinedione anticonvulsants; parathyroid hormone and analogs; penicillinase resistant penicillins; penicillins; peripheral opioid receptor antagonists; peripheral vasodilators; peripherally acting antiobesity agents; phenothiazine antiemetics; phenothiazine antipsychotics; phenylpiperazine antidepressants; plasma expanders; platelet aggregation inhibitors; platelet-stimulating agents; polyenes; potassium-sparing diuretics; probiotics; progesterone receptor modulators; progestins; prolactin inhibitors; prostaglandin D2 antagonists; protease inhibitors; proton pump inhibitors; psoralens; psychotherapeutic agents; psychotherapeutic combinations; purine nucleosides; pyrrolidine anticonvulsants; quinolones; radiocontrast agents; radiologic adjuncts; radiologic agents; radiologic conjugating agents; radiopharmaceuticals; RANK ligand inhibitors; recombinant human erythropoietins; renin inhibitors; respiratory agents; respiratory inhalant products; rifamycin derivatives; salicylates; sclerosing agents; second generation cephalosporins; selective estrogen receptor modulators; selective serotonin reuptake inhibitors; serotonin-norepinephrine reuptake inhibitors; serotoninergic neuroenteric modulators; sex hormone combinations; sex hormones; skeletal muscle relaxant combinations; skeletal muscle relaxants; smoking cessation agents; somatostatin and somatostatin analogs; spermicides; statins; sterile irrigating solutions; streptomyces derivatives; succinimide anticonvulsants; sulfonamides; sulfonylureas; synthetic ovulation stimulants; tetracyclic antidepressants; tetracyclines; therapeutic radiopharmaceuticals; thiazide diuretics; thiazolidinediones; thioxanthenes; third generation cephalosporins; thrombin inhibitors; thrombolytics; thyroid drugs; tocolytic agents; topical acne agents; topical agents; topical anesthetics; topical anti-infectives; topical antibiotics; topical antifungals; topical antihistamines; topical antipsoriatics; topical antivirals; topical astringents; topical debriding agents; topical depigmenting agents; topical emollients; topical keratolytics; topical steroids; topical steroids with anti-infectives; toxoids; triazine anticonvulsants; tricyclic antidepressants; trifunctional monoclonal antibodies; tumor necrosis factor (TNF) inhibitors; tyrosine kinase inhibitors; ultrasound contrast media; upper respiratory combinations; urea anticonvulsants; urinary anti-infectives; urinary antispasmodics; urinary pH modifiers; uterotonic agents; vaccine; vaccine combinations; vaginal anti-infectives; vaginal preparations; vasodilators; vasopressin antagonists; vasopressors; VEGFNEGFR inhibitors; viral vaccines; viscosupplementation agents; vitamin and mineral combinations; vitamins

### Diagnostic Tests

17-Hydroxyprogesterone; ACE (Angiotensin I converting enzyme); Acetaminophen; Acid phosphatase; ACTH; Activated clotting time; Activated protein C resistance; Adrenocorticotropic hormone (ACTH); Alanine aminotransferase (ALT); Albumin; Aldolase; Aldosterone; Alkaline phosphatase; Alkaline phosphatase (ALP); Alpha1-antitrypsin; Alpha-fetoprotein; Alpha-fetoprotien; Ammonia levels; Amylase; ANA (antinuclear antibodies); ANA (antinuclear antibodies); Angiotensin-converting enzyme (ACE); Anion gap; Anticardiolipin antibody; Anticardiolipin antibodies (ACA); Anti-centromere antibody; Antidiuretic hormone; Anti-DNA; Anti-Dnase-B; Anti-Gliadin antibody; Anti-glomerular basement membrane antibody; Anti-HBc (Hepatitis B core antibodies; Anti-HBs (Hepatitis B surface antibody; Antiphospholipid antibody; Anti-RNA polymerase; Anti-Smith (Sm) antibodies; Anti-Smooth Muscle antibody; Antistreptolysin 0 (ASO); Antithrombin Ill; Anti-Xa activity; Anti-Xa assay; Apolipoproteins; Arsenic; Aspartate aminotransferase (AST); B12; Basophil; Beta-2-Microglobulin; Beta-hydroxybutyrate; B-HCG; Bilirubin; Bilirubin, direct; Bilirubin, indirect; Bilirubin, total; Bleeding time; Blood gases (arterial); Blood urea nitrogen (BUN); BUN; BUN (blood urea nitrogen); CA 125; CA 15-3; CA 19-9; Calcitonin; Calcium; Calcium (ionized); Carbon monoxide (CO); Carcinoembryonic antigen (CEA); CBC; CEA; CEA (carcinoembryonic antigen); Ceruloplasmin; CH50Chloride; Cholesterol; Cholesterol, HDL; Clot lysis time; Clot retraction time; CMP; CO2; Cold agglutinins; Complement C3; Copper; Corticotrophin releasing hormone (CRH) stimulation test; Cortisol; Cortrosyn stimulation test; C-peptide; CPK (Total); CPK-MB; C-reactive protein; Creatinine; Creatinine kinase (CK); Cryoglobulins; DAT (Direct antiglobulin test); D-Dimer; Dexamethasone suppression test; DHEA-S; Dilute Russell viper venom; Elliptocytes; Eosinophil; Erythrocyte sedimentation rate (ESR); Estradiol; Estriol; Ethanol; Ethylene glycol; Euglobulin lysis; Factor V Leiden; Factor VIII inhibitor; Factor VIII level; Ferritin; Fibrin split products; Fibrinogen; Folate; Folate (serum; Fractional excretion of sodium (FENA); FSH (follicle stimulating factor); FTA-ABS; Gamma glutamyl transferase (GGT); Gastrin; GGTP (Gamma glutamyl transferase); Glucose; Growth hormone; Haptoglobin; HBeAg (Hepatitis Be antigen); HBs-Ag (Hepatitis B surface antigen); Helicobacter pylori; Hematocrit; Hematocrit (HCT); Hemoglobin; Hemoglobin AlC; Hemoglobin electrophoresis; Hepatitis A antibodies; Hepatitis C antibodies; IAT (Indirect antiglobulin test); Immunofixation (IFE); Iron; Lactate dehydrogenase (LDH); Lactic acid (lactate); LDH; LH (Leutinizing hormone; Lipase; Lupus anticoagulant; Lymphocyte; Magnesium; MCH (mean corpuscular hemoglobin; MCHC (mean corpuscular hemoglobin concentration); MCV (mean corpuscular volume); Methylmalonate; Monocyte; MPV (mean platelet volume); Myoglobin; Neutrophil; Parathyroid hormone (PTH); Phosphorus; Platelets (pit); Potassium; Prealbumin; Prolactin; Prostate specific antigen (PSA); Protein C; Protein S; PSA (prostate specific antigen); PT (Prothrombin time); PTT (Partial thromboplastin time); RDW (red cell distribution width); Renin; Rennin; Reticulocyte count; reticulocytes; Rheumatoid factor (RF); Sed Rate; Serum glutamic-pyruvic transaminase (SGPT; Serum protein electrophoresis (SPEP); Sodium; T3-resin uptake (T3RU); T4, Free; Thrombin time; Thyroid stimulating hormone (TSH); Thyroxine (T4); Total iron binding capacity (TIBC); Total protein; Transferrin; Transferrin saturation; Triglyceride (TG); Troponin; Uric acid; Vitamin B12; White blood cells (WBC); Widal test.

### BRIEF DESCRIPTION OF THE DRAWINGS

FIG. 1 is a schematic sectional view of a vessel according to any embodiment of the invention.

FIG. 2 is an enlarged detail view of a portion of the vessel wall and coatings of FIG. 1.

FIG. 3 is a schematic view of a pharmaceutical package in the form of a syringe barrel as the vessel of FIGS. 1 and 2, containing a fluid and closed with a closure in the form of a plunger.

FIG. 4 is a schematic view of a pharmaceutical package in the form of a vial as the vessel of FIGS. 1 and 2 containing a fluid and closed with a closure.

FIG. 5 is a schematic view of a pharmaceutical package in the form of a blister package as the vessel of FIGS. 1 and 2 containing a fluid and closed with a closure in the form of a coated sheet defining an additional vessel wall.

In the context of the present invention, the following definitions and abbreviations are used:

“Drug product” refers to a composition, typically a fluid, containing a pharmacologically active substance (also referred to as an active pharmaceutical ingredient or API) and optionally one or more excipients. A reduction in the rate and/or amount of degradation of a drug product includes a reduction in the rate and/or amount of degradation of the pharmaceutically active substance as well as a reduction in the rate and/or amount of degradation of the one or more of excipients. For instance, a reduction in the rate and/or amount of degradation of a drug product may include either a reduction solely in the rate and/or amount of degradation of the pharmaceutically active substance or a reduction solely in the rate and/or amount of degradation of the one or more excipients. A reduction in the rate and/or amount of degradation of a drug product may also include both a reduction in the rate and/or amount of degradation of the pharmaceutically active substance and a reduction in the rate and/or amount of degradation of the one or more excipients.

“Excipient” refers to any pharmacologically inactive substance that, when combined with a pharmacologically active substance, provides a benefit to the drug product. These benefits may include, for instance, (a) enhancing solubility of the active substance, (b) enhancing process and/or shelf life stability of the active substance, (c) controlling pH and tonicity of the composition, (d) maintaining a preferred stable conformation for active proteins or vaccines, including exposure of the functional epitopes, (e) preventing aggregation or degradation of the active substance, (f) enhancing the pharmacological effect of the active substance or increasing the ability of an antigen to stimulate the immune system, e.g., an adjuvant, and (g) one or more of several other functions including but not limited to bulking agents, antioxidants, colorants, and preservatives. Due to the complexity and fragility of biologic drugs, excipients are of particular importance for biological drug products, e.g. to increase product stability, maintain tonicity, and/or facilitate drug delivery.

Common excipients include buffering agents (pH modifiers) such as acetate, citrate, citric acid, sodium citrate, tartrate, histidine, glutamate, phosphate, tris(hydroxymethyl)aminomethane (“Tris”), glycine, bicarbonate, succinate, sulfate, and nitrate; tonicity modifiers such as mannitol, sorbitol, lactose, dextrose, trehalose, sucrose, sodium chloride, potassium chloride, glycerol, and glycerine; bulking agents such as arginine, aspartic acid, glutamic acid, lysine, proline, glycine, histidine, methionine, alanine, gelatin, PVP, PLGA, PEG, dextran, cyclodextrin and derivatives, starch derivatives, HSA, and BSA; surfactants (wetting and/or solubilizing agents) such as polysorbates (e.g. polysorbate 20 and polysorbate 80), poloxamers (e.g. Pluronic F68 and F127), Triton X-100, Brij 30, Brij 35, and sodium lauryl sulfate; antioxidant preservatives such as histamine, cysteine, methionine, ascorbic acid, glutathione, vitamin E, vitamin A, propyl gallate, retinyl palmitate, selenium, and poly(ethylenimine); antimicrobial preservatives such as benzyl alcohol, metacresol, phenol, 2-phenoxyethanol, and parabens (e.g. methyl paraben and propyl paraben); chelating and/or complexing agents (preservatives) such as edetate disodium, diethylenetriamine pentaacetic acid (DTPA), citric acid, hexaphosphate, thioglycolic acid, and zinc; adjuvants; and colorants. In particular, sodium chloride, polysorbate (e.g. polysorbate 20 or polysorbate 80), sucrose, and mannitol are present as excipients in many drug products.

RF is radio frequency.

The term “at least” in the context of the present invention means “equal or more” than the integer following the term. The word “comprising” does not exclude other elements or steps, and the indefinite article “a” or “an” does not exclude a plurality unless indicated otherwise. Whenever a parameter range is indicated, it is intended to disclose the parameter values given as limits of the range and all values of the parameter falling within said range.

“First” and “second” or similar references to, for example, deposits of lubricant, processing stations or processing devices refer to the minimum number of deposits, processing stations or devices that are present, but do not necessarily represent the order or total number of deposits, processing stations and devices or require additional deposits, processing stations and devices beyond the stated number. These terms do not limit the number of processing stations or the particular processing carried out at the respective stations. For example, a “first” deposit in the context of this specification can be either the only deposit or any one of plural deposits, without limitation. In other words, recitation of a “first” deposit allows but does not require an embodiment that also has a second or further deposit.

For purposes of the present invention, an “organosilicon precursor” is a compound having at least one of the linkages:

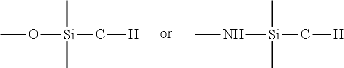

which is a tetravalent silicon atom connected to an oxygen or nitrogen atom and an organic carbon atom (an organic carbon atom being a carbon atom bonded to at least one hydrogen atom). A volatile organosilicon precursor, defined as such a precursor that can be supplied as a vapor in a PECVD apparatus, is an optional organosilicon precursor. Optionally, the organosilicon precursor is selected from the group consisting of a linear siloxane, a monocyclic siloxane, a polycyclic siloxane, a polysilsesquioxane, an alkyl trimethoxysilane, a linear silazane, a monocyclic silazane, a polycyclic silazane, a polysilsesquiazane, and a combination of any two or more of these precursors.

The feed amounts of PECVD precursors, gaseous reactant or process gases, and carrier gas are sometimes expressed in “standard volumes” in the specification and claims. The standard volume of a charge or other fixed amount of gas is the volume the fixed amount of the gas would occupy at a standard temperature and pressure (without regard to the actual temperature and pressure of delivery). Standard volumes can be measured using different units of volume, and still be within the scope of the present disclosure and claims. For example, the same fixed amount of gas could be expressed as the number of standard cubic centimeters, the number of standard cubic meters, or the number of standard cubic feet. Standard volumes can also be defined using different standard temperatures and pressures, and still be within the scope of the present disclosure and claims. For example, the standard temperature might be 0° C. and the standard pressure might be 760 Torr (as is conventional), or the standard temperature might be 20° C. and the standard pressure might be 1 Torr. But whatever standard is used in a given case, when comparing relative amounts of two or more different gases without specifying particular parameters, the same units of volume, standard temperature, and standard pressure are to be used relative to each gas, unless otherwise indicated.

The corresponding feed rates of PECVD precursors, gaseous reactant or process gases, and carrier gas are expressed in standard volumes per unit of time in the specification. For example, in the working examples the flow rates are expressed as standard cubic centimeters per minute, abbreviated as sccm. As with the other parameters, other units of time can be used, such as seconds or hours, but consistent parameters are to be used when comparing the flow rates of two or more gases, unless otherwise indicated.

A “vessel” in the context of the present invention can be any type of vessel with at least one opening and a wall defining an inner or interior surface. The substrate can be the wall of a vessel having a lumen. Though the invention is not necessarily limited to pharmaceutical packages or other vessels of a particular volume, pharmaceutical packages or other vessels are contemplated in which the lumen has a void volume of from 0.5 to 50 mL, optionally from 1 to 10 mL, optionally from 0.5 to 5 mL, optionally from 1 to 3 mL. The substrate surface can be part or all of the inner or interior surface of a vessel having at least one opening and an inner or interior surface. Some examples of a pharmaceutical package include, but are not limited to, a vial, a plastic-coated vial, a syringe, a plastic coated syringe, a blister pack, an ampoule, a plastic coated ampoule, a cartridge, a bottle, a plastic coated bottle, a pouch, a pump, a sprayer, a stopper, a needle, a plunger, a cap, a stent, a catheter or an implant.

The term “at least” in the context of the present invention means “equal or more” than the integer following the term. Thus, a vessel in the context of the present invention has one or more openings. One or two openings, like the openings of a sample tube (one opening) or a syringe barrel (two openings) are preferred. If the vessel has two openings, they can be of same or different size. If there is more than one opening, one opening can be used for the gas inlet for a PECVD coating method according to the present invention, while the other openings are either capped or open. A vessel according to the present invention can be a sample tube, for example for collecting or storing biological fluids like blood or urine, a syringe (or a part thereof, for example a syringe barrel) for storing or delivering a biologically active compound or composition, for example a medicament or pharmaceutical composition, a vial for storing biological materials or biologically active compounds or compositions, a pipe, for example a catheter for transporting biological materials or biologically active compounds or compositions, or a cuvette for holding fluids, for example for holding biological materials or biologically active compounds or compositions.

A vessel can be of any shape, a vessel having a substantially cylindrical wall adjacent to at least one of its open ends being preferred. Generally, the interior wall of the vessel is cylindrically shaped, like, for example in a sample tube or a syringe barrel. Sample tubes and syringes or their parts (for example syringe barrels) are contemplated.

A “hydrophobic layer” in the context of the present invention means that the coating or layer lowers the wetting tension of a surface coated with the coating or layer, compared to the corresponding uncoated surface. Hydrophobicity is thus a function of both the uncoated substrate and the coating or layer. The same applies with appropriate alterations for other contexts wherein the term “hydrophobic” is used. The term “hydrophilic” means the opposite, i.e. that the wetting tension is increased compared to reference sample. The present hydrophobic layers are primarily defined by their hydrophobicity and the process conditions providing hydrophobicity

These values of w, x, y, and z are applicable to the empirical composition Si_{w}OₓC_{y}H_{z} throughout this specification. The values of w, x, y, and z used throughout this specification should be understood as ratios or an empirical formula (for example for a coating or layer), rather than as a limit on the number or type of atoms in a molecule. For example, octamethylcyclotetrasiloxane, which has the molecular composition Si₄O₄C₈H₂₄, can be described by the following empirical formula, arrived at by dividing each of w, x, y, and z in the molecular formula by 4, the largest common factor: Si₁O₁C₂H₈. The values of w, x, y, and z are also not limited to integers. For example, (acyclic) octamethyltrisiloxane, molecular composition Si₃O₂C₈H₂₄, is reducible to Si₁O_{0.67}C_{2.67}H₈. Also, although SiOₓC_{y}H_{z} is described as equivalent to SiOₓC_{y}, it is not necessary to show the presence of hydrogen in any proportion to show the presence of SiOₓC_{y}.

“Wetting tension” is a specific measure for the hydrophobicity or hydrophilicity of a surface. An optional wetting tension measurement method in the context of the present invention is ASTM D 2578 or a modification of the method described in ASTM D 2578. This method uses standard wetting tension solutions (called dyne solutions) to determine the solution that comes nearest to wetting a plastic film surface for exactly two seconds. This is the film's wetting tension. The procedure utilized is varied herein from ASTM D 2578 in that the substrates are not flat plastic films, but are tubes made according to the Protocol for Forming PET Tube and (except for controls) coated according to the Protocol for coating Tube Interior with Hydrophobic Coating or Layer (see Example 9 of EP2251671 A2).

The atomic ratio can be determined by XPS. Taking into account the H atoms, which are not measured by XPS, the coating or layer may thus in one aspect have the formula Si_{w}OₓC_{y}H_{z} (or its equivalent SiOₓC_{y}), for example where w is 1, x is from about 0.5 to about 2.4, y is from about 0.6 to about 3, and z is from about 2 to about 9. Typically, such coating or layer would hence contain 36% to 41% carbon normalized to 100% carbon plus oxygen plus silicon.

The term “syringe” is broadly defined to include cartridges, injection “pens,” and other types of barrels or reservoirs adapted to be assembled with one or more other components to provide a functional syringe. “Syringe” is also broadly defined to include related articles such as auto-injectors, which provide a mechanism for dispensing the contents.

A coating or layer or treatment is defined as “hydrophobic” if it lowers the wetting tension of a surface, compared to the corresponding uncoated or untreated surface. Hydrophobicity is thus a function of both the untreated substrate and the treatment.

A “lubricity layer” according to the present invention is a coating which has a lower frictional resistance than the uncoated surface. In other words, it reduces the frictional resistance of the coated surface in comparison to a reference surface which is uncoated. The present lubricity layers are primarily defined by their lower frictional resistance than the uncoated surface and the process conditions providing lower frictional resistance than the uncoated surface, and optionally can have a composition according to the empirical composition Si_{w}OₓC_{y}H_{z}, as defined in this Definition Section. “Frictional resistance” can be static frictional resistance and/or kinetic frictional resistance. One of the optional embodiments of the present invention is a syringe part, e.g. a syringe barrel or plunger, coated with a lubricity layer. In this contemplated embodiment, the relevant static frictional resistance in the context of the present invention is the breakout force as defined herein, and the relevant kinetic frictional resistance in the context of the present invention is the plunger sliding force as defined herein. For example, the plunger sliding force as defined and determined herein is suitable to determine the presence or absence and the lubricity characteristics of a lubricity layer in the context of the present invention whenever the coating is applied to any syringe or syringe part, for example to the inner wall of a syringe barrel. The breakout force is of particular relevance for evaluation of the coating effect on a prefilled syringe, i.e. a syringe which is filled after coating and can be stored for some time, e.g. several months or even years, before the plunger is moved again (has to be “broken out”).

The “plunger sliding force” in the context of the present invention is the force required to maintain movement of a plunger in a syringe barrel, e.g. during aspiration or dispense. It can advantageously be determined using the ISO 7886-1:1993 test described herein and known in the art. A synonym for “plunger sliding force” often used in the art is “plunger force” or “pushing force”. The “breakout force” in the context of the present invention is the initial force required to move the plunger in a syringe, for example in a prefilled syringe. Both “plunger sliding force” and “breakout force” and methods for their measurement are described in more detail in subsequent parts of this description. “Slidably” means that the plunger is permitted to slide in a syringe barrel.

In the context of this invention, “substantially rigid” means that the assembled components (ports, duct, and housing, explained further below) can be moved as a unit by handling the housing, without significant displacement of any of the assembled components respecting the others. Specifically, none of the components are connected by hoses or the like that allow substantial relative movement among the parts in normal use. The provision of a substantially rigid relation of these parts allows the location of the vessel seated on the vessel holder to be nearly as well known and precise as the locations of these parts secured to the housing.

The word “comprising” does not exclude other elements or steps.

The indefinite article “a” or “an” does not exclude a plurality.

### DETAILED DESCRIPTION

The present invention will now be described more fully, with reference to the accompanying drawings, in which several embodiments are shown. This invention can, however, be embodied in many different forms and should not be construed as limited to the embodiments set forth here. Rather, these embodiments are examples of the invention, which has the full scope indicated by the language of the claims. Like numbers refer to like or corresponding elements throughout. The following disclosure relates to all embodiments unless specifically limited to a certain embodiment.

Polysorbate degradation results in the accumulation of free fatty acids (FFA), which ultimately precipitate to form particles upon long-term storage. Thus, the amount of polysorbate in a solution may be quantified by analyzing the presence of different FFA in solution at various points in time over an extended storage period. The amount of different FFA in the solution can be measured using any of a variety of techniques, including for example FTIR, dispersive Raman spectroscopy, Raman microscopy, mixed mode chromatography, liquid chromatography charged aerosol detection, and reverse phase ultra high performance liquid chromatography (RP UHPLC), e.g. after derivatization with PDAM.

In embodiments of the present disclosure, the FFA profile of a sample may be measured using the RP UHPLC technique described by Tomlinson et al. in “Polysorbate 20 Degradation in Biopharmaceutical Formulations: Quantification of Free Fatty Acids, Characterization of Particulates and Insights into the Degradation Mechanism,” published in Molecular Pharmaceutics, vol. 12, 2805-3815 (2015), the entirety of which is incorporated herein by reference.

The free fatty acids that can be measured include, but are not limited to, any one or more of the following: caprylic acid, capric acid, lauric acid, myristic acid, linoleic acid (or myristic/linoleic acid), palmitic acid, oleic acid (or palmitic/oleic acid), and stearic acid. In some embodiments, the concentration of lauric acid may be measured over time and correlated with the degradation of polysorbate, e.g. polysorbate 20 or polysorbate 80. In some embodiments, the concentrations of lauric acid, myristic acid, and palmitic acid may be measured over time and correlated with the degradation of polysorbate, e.g. polysorbate 20 or polysorbate 80.

Due to the molecular heterogeneity of polysorbate and the interference from proteins and the excipient in the formulation, the study of polysorbate degradation in drug solutions has proven challenging. To reduce this interference, the proteins and/or excipients may be removed prior to testing.

The amount of polysorbate in an aqueous drug-containing solution can also be measured directly. For instance, the amount of polysorbate in an aqueous drug-containing solution can be measured by a spectrophotometric method described by Justin Kim et al. in “Quantitation of low concentrations of polysorbates in high protein concentration formulations by solid phase extraction and cobalt-thiocyanate derivatization,” published in Analytica Chimica Acta, vol. 806, pages 144-151 (January 2014), the entirety of which is incorporated by reference herein. In particular, Kim et al. describe a spectrophotometric method to quantify low polysorbate levels in biopharmaceutical formulations containing high protein concentrations. In the method, Oasis HLB solid phase extraction (SPE) cartridges are used to extract polysorbate from high protein concentration formulations. After loading a sample, the cartridge is washed with 4M guanidine HCl and 10% (v/v) methanol, and the retained polysorbate is eluted by acetonitrile. Following the evaporation of acetonitrile, aqueous cobalt-thiocyanate reagent is added to react with the polyoxyethylene oxide chain of the polysorbate to form a blue colored polysorbate-cobaltothiocyante complex. This colored complex is then extracted into methylene chloride and measured spectrophotometrically at 620 nm. By diluting samples with 6M guanidine HCl and/or using different methylene chloride volumes to extract the colored complexes of standards and samples, Kim et al. found that the method could accurately and precisely quantify 40 mg/L polysorbate in up to 300 g/L protein formulations.

In previous studies, both mAb-A (at a pH of about 5) and mAb-B (at a pH of about 6) liquid drug products showed about 20% polysorbate 20 (PS20) degradation over their respective shelf lives (at 2-8° C.). Commercially available PS20 contains about 40-60% lauric acid ester species, 14-25% myristic acid ester species, and 7-15% palmitic acid ester species. Assuming that 1 mol of lauric acid is produced for every 1 mol of PS20 that degrades, a free lauric acid concentration of about 8 pg/mL is expected at end of shelf life for a formulation with 0.04% (w/v) PS20 and a free lauric acid concentration of about 4 pg/mL is expected at end of shelf life for a formulation with 0.02% (w/v) PS20. Similarly, assuming that 1 mol of myristic acid is produced for every 1 mol of PS20 that degrades, a free myristic acid concentration of about 4 pg/mL is expected at end of shelf life for a formulation with 0.04% (w/v) PS20 and a free myristic acid concentration of about 2 pg/mL is expected at end of shelf life for a formulation with 0.02% (w/v) PS20. Similarly, assuming that 1 mol of palmitic acid is produced for every 1 mol of PS20 that degrades, a free palmitic acid concentration of about 2 pg/mL is expected at end of shelf life for a formulation with 0.04% (w/v) PS20 and a free palmitic acid concentration of about 1 pg/mL is expected at end of shelf life for a formulation with 0.02% (w/v) PS20.

By using embodiments of the coatings described herein, it is believed that one may achieve free fatty acid contents significantly lower than the expected values recited above, optionally by at least 5%, optionally by at least 10%, optionally by at least 15%, optionally by at least 20%, optionally by at least 25%, optionally by at least 30%, optionally by at least 35%, optionally by at least 40%, optionally by at least 45%, optionally by at least 50%, optionally by at least 60%, optionally by at least 70%, optionally by at least 80%, optionally by at least 90%.

In some embodiments, the polysorbate-containing drug solution may contain between 0.01 mg/mL and 2 mg/mL, alternatively between 0.01 mg/mL and 1 mg/mL, alternatively between 0.01 mg/mL and 0.9 mg/mL, alternatively between 0.01 mg/mL and 0.8 mg/mL, alternatively between 0.01 mg/mL and 0.7 mg/mL, alternatively between 0.01 mg/mL and 0.6 mg/mL, alternatively between 0.01 mg/mL and 0.5 mg/mL, alternatively between 0.01 mg/mL and 0.4 mg/mL, alternatively between 0.01 mg/mL and 0.3 mg/mL, alternatively between 0.01 mg/mL and 0.2 mg/mL, alternatively between 0.01 mg/mL and 0.1 mg/mL, alternatively between 0.01 mg/mL and 0.09 mg/mL, alternatively between 0.01 mg/mL and 0.08 mg/mL, alternatively between 0.01 mg/mL and 0.07 mg/mL. In some embodiments, the polysorbate-containing drug solution may contain between 0.01 and 0.09% w/v polysorbate, alternatively between 0.01% and 0.08% w/v polysorbate, alternatively between 0.01% and 0.06% w/v polysorbate, alternatively between 0.01% and 0.05% w/v polysorbate, alternatively between 0.01% and 0.04% w/v polysorbate, alternatively between 0.02% and 0.08% w/v polysorbate, alternatively between 0.02% and 0.07% w/v polysorbate, alternatively between 0.02% and 0.06% w/v polysorbate, alternatively between 0.02% and 0.05% w/v polysorbate, alternatively between 0.02% and 0.04% w/v polysorbate.

In some embodiments, the polysorbate-containing drug solution may have a pH in the range of 3 to 14, alternatively 5 to 14, alternatively 5 to 9, alternatively 5 to 8, alternatively 5 to 7. In some embodiments, the polysorbate-containing drug solution may be stored at 25° C. or less than 25° C., for instance between 2° C. and 8° C., alternatively at about 5° C., alternatively at about 4° C.

The results of polysorbate testing that involved the measurement of free fatty acid degradation products to determine the degree of polysorbate degradation, demonstrated that after three months of storage at 25° C. in the lumen of a syringe coated with a quadlayer coating of the sort described herein—namely a trilayer coating having an OMCTS-based lubricity layer as a top layer, an aqueous solution containing polysorbate appears to have been essentially free from polysorbate degradation. Similarly, the results of polysorbate testing that involved the measurement of free fatty acid degradation products to determine the degree of polysorbate degradation, demonstrated that after one month of storage at 40° C. in the lumen of a syringe coated with a quadlayer coating of the sort described herein—namely a trilayer coating having an OMCTS-based lubricity layer as a top layer, an aqueous solution containing polysorbate appears to have been essentially free from polysorbate degradation.

The coating or layer effective to reduce degradation of the drug product may comprise, consist essentially of, or consist of organosiloxanes and organosilazanes, including but not limited to Si_{w}OₓC_{y}H (Si_{w}OₓC_{y}H_{z}) or Si_{w}NₓC_{y}H (Si_{w}NₓC_{y}H_{z}) as described herein; silicon nitrides including but not limited to Si₃N₄; diamond-like carbon (DLC); amorphous carbon (a:C-H); fluorocarbons (a:C-F); pegylated carbon; polysaccharide-derived carbon; and glyme-derived carbon. The coating or layer effective to reduce degradation of the drug product may be applied by PECVD or atomic layer deposition (ALD).

One or more of the coatings or layers described herein may be applied by atomic layer deposition coating. Coatings applied by atomic layer deposition are structurally (though not necessarily chemically) distinct from those applied by CVD or PECVD. In contrast to coatings applied by CVD or PECVD, coatings applied by atomic layer deposition consist of a plurality of monolayers of the deposited compound. Because each step deposited only a single monolayer, defects of the sort that can develop due to non-uniform growth during CVD or PECVD are avoided. The result is a coating having significantly higher density than that of a coating (of generally the same chemical composition) applied by CVD or PECVD. Because the coating consists of a plurality of monolayers of the deposited compound, the coating may also have a higher degree of compositional purity and consistency than coatings applied by PECVD.

It is believed that a coating of the sort described herein may surprisingly be effective to reduce the degradation of polysorbates in a solution that is stored in contact with the coated surface over time by preventing or reducing metal leaching, also known as ion exchange, into the drug-containing solution.

Type 1 borosilicate glass is composed of about 75-80% SiO₂, about 10% B₂O₃ and the remainder are metals. For instance, lithium (Li₂O), Sodium (Na₂O), Potassium (K₂O), Magnesium (MgO), Barium (BaO), Calcium (CaO) may be added as network modifiers, e.g. to bring electronic charge and/or extra oxygen to the cross-linked network of SiO₂ and B₂O₃, to reduce the glass melting point and/or viscosity, etc. Intermediate oxides, such as ZnO and PbO, cannot form a glass structure on their own, but may be added to assist the building of the network structure by aiding other oxides. Further, Fe₂O₃, Ti₂O₃ and/or MnO may be added into borosilicate glasses to produce amber color, which provide protection from ultraviolet light. A typical Type I borosilicate glass container for parenteral use, for instance, may be composed of 80% SiO₂, 10% B₂O₃, and a small amount of Na₂O and Al₂O₃. These metals are intended to be uniformly distributed throughout the glass matrix. However, during the glass forming process, these metals can migrate to the surface of the glass vessel, particularly in the case of vials. These surface metals are more likely to leach into the drug. At least Si, B, Na, Al, Ca, K, Fe, Mn, and As have been detected in liquid drug formulations stored in Type 1 borosilicate glass containers. The common metal ion contaminants from glass surfaces include Al³⁺, Fe^{3+/2+}, Ca²⁺, Ba²⁺, Mn²⁺, and Zn²⁺.

This leaching (or ion exchange process) occurs more rapidly in lower pH (more acidic) formulations.

Similarly, many plastics are made by polymerization processes that utilize metal catalysts. The resulting plastics may thus contain metal catalyst residuals that can leach from a drug solution contacting surface into the drug solution. Without being bound by theory, it is believed that the leaching of metal ions into a drug-containing solution may be at least partially responsible for polysorbate degradation.

Accordingly, embodiments of the present disclosure are directed to vessels having the interior surface that faces a lumen in which a drug composition comprising a polysorbate is stored, in which the interior surface is provided with a coating that is free of metal ions or substantially free of metals and metal ions (by substantially free, it is meant that the amount of metals and/or metal ions present in the coating is so low as to have no effect on the resulting polysorbate degradation properties relative to a coating that is completely free of metals and metal ions). These coatings are configured so that the leaching of metal ions into the drug composition is avoided.

In some embodiments, these coatings may be applied to borosilicate glass vessels or thermoplastic vessels (e.g. COP or COC vessels) in order to protect the drug composition from metals present within the vessel walls. These coatings may also be applied to vessels in which the interior surface of the wall has been treated by one or more additional coatings, e.g. a gas barrier coating such as one that comprises one or more metal oxides. In both cases, the coating may serve to prevent metals present within the vessel walls from leaching into the drug composition that is stored within the vessel lumen.

Embodiments of the metal-free coatings may be configured to withstand dissolution by the drug-containing solution. Though ion exchange is accelerated under more acidic conditions, i.e. where the drug-containing solution has a relatively low pH, ion exchange will still occur under substantially any pH conditions. Moreover, many coatings such as SiO₂ or metal oxide coatings are readily dissolved under more basic conditions, i.e. where the drug-containing solution has a relatively high pH. Thus, while a SiO₂ coating may be capable of preventing ion exchange at very low pHs (e.g. a pH of 3 or less), such a coating would be quickly dissolved by drug-containing solutions having a pH of about 5 or higher. Embodiments of the metal-free coatings described herein, e.g. organosilicon coatings, may be configured to withstand solutions having a pH of about 5 or higher, thereby providing protection against metal leaching for a longer period of time or shelf life. Due to their ability to withstand dissolution by higher pH solutions, embodiments of the metal-free coatings described herein may effectively be used to protect more/different drug products from metal ion leaching—and degradation of the drug product caused by metal ion leachables—than a SiO₂ coating.

In some embodiments, for example, the metal-free coatings described herein, e.g. organosilicon coatings, may be configured to prevent ion exchange (i.e. metal ion leaching) in solutions having pHs between 2 and 9, while also withstanding silicon dissolution across that same range of pHs. Similarly, the metal-free coatings described herein, e.g. organosilicon coatings, may be configured to prevent ion exchange (i.e. metal ion leaching) in solutions having pHs between 2 and 8, while also withstanding silicon dissolution across that same range of pHs. Similarly, the metal-free coatings described herein, e.g. organosilicon coatings, may be configured to prevent ion exchange (i.e. metal ion leaching) in solutions having pHs between 2 and 7, while also withstanding silicon dissolution across that same range of pHs.

For example, in some embodiments, the metal-free coating (e.g. organosilicon coating) may be configured so that a fluid having a pH of 5 removes the coating at a rate of 1 nm or less of coating thickness per 44 hours of contact, alternatively a rate of 1 nm or less of coating thickness per 88 hours of contact, alternatively a rate of 1 nm or less of coating thickness per 350 hours of contact. In some embodiments, the metal-free coating (e.g. organosilicon coating) may be configured so that a fluid having a pH of 6 removes the coating at a rate of 1 nm or less of coating thickness per 44 hours of contact, alternatively a rate of 1 nm or less of coating thickness per 88 hours of contact, alternatively a rate of 1 nm or less of coating thickness per 350 hours of contact. In some embodiments, the metal-free coating (e.g. organosilicon coating) may be configured so that a fluid having a pH of 7 removes the coating at a rate of 1 nm or less of coating thickness per 44 hours of contact, alternatively a rate of 1 nm or less of coating thickness per 88 hours of contact, alternatively a rate of 1 nm or less of coating thickness per 350 hours of contact. In some embodiments, the metal-free coating (e.g. organosilicon coating) may be configured so that a fluid having a pH of 8 removes the coating at a rate of 1 nm or less of coating thickness per 44 hours of contact, alternatively a rate of 1 nm or less of coating thickness per 88 hours of contact, alternatively a rate of 1 nm or less of coating thickness per 350 hours of contact. In some embodiments, the metal-free coating (e.g. organosilicon coating) may be configured so that a fluid having a pH of 9 removes the coating at a rate of 1 nm or less of coating thickness per 44 hours of contact, alternatively a rate of 1 nm or less of coating thickness per 88 hours of contact, alternatively a rate of 1 nm or less of coating thickness per 350 hours of contact.

Similarly, the metal-free coating may be configured such that the silicon dissolution rate by a 50 mM potassium phosphate buffer diluted in water, adjusted to pH 8 with concentrated nitric acid, and containing 0.2 wt. % polysorbate-80 surfactant, at 40° C., is less than 170 ppb/day, alternatively less than 160 ppb/day, alternatively less than 140 ppb/day, alternatively less than 120 ppb/day, alternatively less than 100 ppb/day, alternatively less than 90 ppb/day, alternatively less than 80 ppb/day. In contrast, the silicon dissolution rate of a coating of SiO₂ would be much higher.

Embodiments of the metal-free coatings may also be configured to operate as a lubricity coating or layer, as described herein.

In addition to or as an alternative to reducing polysorbate degradation, embodiments of the metal-free coatings described herein may more generally reduce or prevent metal leachables from entering the drug product. Contamination of drug formulations by metal leachables could have a profound negative impact on the safety, stability and efficacy of the drug product, and may even induce acute toxicity or long-term health problems.

Although metal leachables could affect any drug product, some drug formulations tend to be more vulnerable to metal leachable than the others. It is often due to the formulation buffer, pH of the formulation, and/or properties of the drug product.

It has been shown for instance that bisphosphonates (such as zoledronic acid and minodronic acid) are highly sensitive to di- and polyvalent cations, and could interact with calcium, barium, magnesium, aluminum, and boron present in glass containers. Parenteral nutrition formulations, which often contain amino acids such as cysteine, cysteine, aspartic acid, glutamic acid, glucose, etc., may suffer from contamination from metal ions, including aluminum and arsenic. Antibiotics, and in particular micronomicin solutions, have been found to have high levels of barium sulphate crystals, which are believed to be the result of interactions with Ba ions from glass containers. For photolabile formulations, tinted glass containers are often chosen to protect them from light, and Fe₂O₃, Ti₂O₃ and MnO are often added into the glass to produce the amber color. Surprisingly, instead of being stable, some photosensitive formulations may be degraded even quicker in such colored containers due to a free radical-mediated oxidation process enhanced by the presence of Fe ion contaminants leached from the colored glass.

In particular, biological formulations, vaccines, and radiopharmaceutical products may be particularly susceptible to metal ion leachables. Many biologic formulations are high ionic strength and/or include surfactants that may enhance the risk of leaching. Furthermore, many biologic products are protein formulations. Proteins are often large molecules with extensive surface area, and stabilize through noncovalent interactions, which makes them vulnerable to molecular modifications based on environmental changes. Inadvertent contamination with metal ions could cause the degradation of proteins. The contaminants may reduce stability and efficacy of those drug products, such as monoclonal antibody and human relaxin. The deleterious changes of the drug could also cause health problems in patients. The mechanisms that contribute to the metal-ion induced protein degradation include protein fragmentation, protein aggregation, and insoluble particle formation.

The leaching of metal ions into mineral adjuvant vaccine formulations may have a destabilizing effect on vaccine stability. High amounts of residual metal ions could interact with mineral adjuvant in the drug product, which leads to free radical formation. These could in turn react with antigen integrity leading to a significantly reduced shelf life of such vaccines. For instance, BioThraxis is a vaccine that contains anthrax antigen filtrate, aluminum hydroxide to adsorb anthrax protective antigen as well as to serve as an adjuvant (immune enhancer), benzethonium chloride as a preservative, formaldehyde as a preservative, sodium chloride as part of a saline solution, and water for injection. BioThrax uses an alanine formulation buffer comprising alanine, sodium phosphate and polysorbate 80, with pH range between about 6.2 and 8.0. BioThrax is quite vulnerable to metal ion leachables, probably because of aluminum hydroxide in the formulation.

A radiopharmaceutical often consists of a radioisotope and optionally biologic molecules as vehicles, and have high affinity for specific organs, tissues or cells within the human body. Radioactive drugs are used as a therapy to treat diseases, or to produce images of organs or tissue for diagnosis of diseases. Leached metal ions could interact with radiopharmaceutical drugs and reduce their efficacy through three different mechanisms: 1) leached metal could interact with the ligand in the original radiopharmaceutical metal complex and replace the radiometal; 2) leached metal could interact with the ligand, which is usually in vast excess over the radiometal and form an impurity, which may tend to precipitate or promote co-precipitation of the desired complex; 3) where the ligand is a chelating agent, binuclear or polynuclear metal complexes involving both radiometal and non-radioactive metal leachable could form. Additionally, since the radiopharmaceutical products usually exist at very low concentration, even low levels of leached metal could have big impact on the product.

### Vessels and Coating Sets

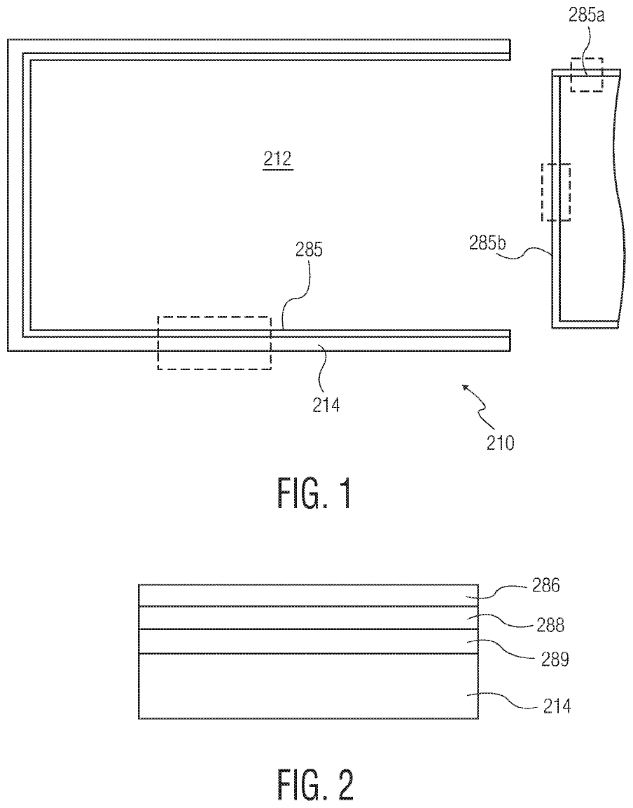

An aspect of the invention, illustrated most broadly by FIG. 1 and the detail view of FIG. 2, is a vessel 210 including a wall 214 enclosing a lumen 212 and a vessel coating or layer set 285 on at least a portion of the wall 214 facing the lumen 212. The vessel may be more specifically a vial, a syringe, a blister pack, an ampoule, a cartridge, a bottle, a pouch, a pump, a sprayer, a stopper, a needle, a plunger, a cap, a stent, a catheter or an implant, or any other type of container or conduit for a fluid. FIGS. #1 through 5 show a vessel having at least a single opening, and should be understood to include a vessel having two or more openings, such as a syringe, or a vessel having no openings, such as a pouch, blister pack, or ampoule.

An embodiment of the vessel coating or layer set 285 is at least one tie coating or layer 289, at least one barrier coating or layer 288, and at least one pH protective coating or layer 286, illustrated in FIGS. #1, #2. This embodiment of the vessel coating or layer set is sometimes known as a “trilayer coating” in which the barrier coating or layer 288 of SiOₓ is protected against contents having a pH otherwise high enough to remove it by being sandwiched between the pH protective coating or layer 286 and the tie coating or layer 289, each an organic layer of SiOₓC_{y} as defined in this specification. A specific example of this trilayer coating is provided in this specification. The contemplated thicknesses of the respective layers in nm (preferred ranges in parentheses) are given in the Trilayer Thickness Table.

Trilayer Thickness Table

| Adhesion | Barrier | Protection |
|---|---|---|
| 5-100 | 20-200 | 50-500 |
| (5-20) | (20-30) | (100-200) |

Several particular coordinating coating sets 285, 285a, and 285b for a vessel 210 and closure of FIG. #1 are shown in the Table of Coating Sets:

Table of Coating Sets

| | Vessel wall | Closure sliding surface | Closure facing surface |
|---|---|---|---|
| Set | (285) | (285a) | (285b) |
| 1 | pH protective (286) | Lubricity (281) e.g. | Barrier (288) - e.g. |
| | barrier (288) | Parylene. | Parylene |
| | tie (289) | Sliding surface of closure, | Facing surface of closure, |
| | syringe barrel wall (214) | e.g. plunger tip | e.g. plunger tip. |
| 2 | Lubricant deposit (287) | No coating set 285a | pH protective (286) |
| | SiOₓ primer (283) | Sliding surface of closure, | barrier (288) |
| | pH protective (286) | e.g. plunger tip | Facing surface of closure |
| | barrier (288) | | |
| | tie (289) | | |
| | syringe barrel wall (214) | | |
| 3 | pH protective (286) | Lubricity (281) e.g. | Barrier (288) - e.g. |
| | barrier (288) | Parylene. | Parylene |
| | syringe barrel wall (214) | Sliding surface of closure, | Facing surface of closure, |
| | | e.g. plunger tip | e.g. plunger tip. |
| 4 | SiOₓ primer (283) | Lubricity (281) e.g. SiOₓC_{y} | pH protective (286) |
| | pH protective (286) | Sliding surface of closure, | barrier (288) |
| | barrier (288) | e.g. plunger tip | Facing surface of closure |
| | syringe barrel wall (214) | | |
| 5 | pH protective (286) | Lubricant deposit (287) | Lubricant deposit (287) |
| | barrier (288) | Sliding surface of closure | Facing surface of closure |
| | Vial wall (214) | (e.g. septum) | (e.g. septum) |
| 6 | pH protective (286) | Lubricant deposit (287) | Lubricant deposit (287) |
| | barrier (288) | Sliding surface of closure | Facing surface of closure |
| | tie (289) | (e.g. septum) | (e.g. septum) |
| | Vial wall (214) | | |
| 7 | Lubricity (281) e.g. SiOₓC_{y} | Barrier (288) - e.g. | Barrier (288) - e.g. |
| | pH protective (286) | Parylene | Parylene |
| | barrier (288) | | |
| | tie (289) | | |
| | Vial wall (214) | | |

Sets 1-4 and 7 in the Table of Coating Sets are among the useful alternatives for a syringe. The syringe barrel wall coatings (left column) of Set 1 are one example of the previously described trilayer coating, and Set 7 is a modification of the trilayer coating in which a PECVD lubricant coating or layer is the top layer of the set.

The Set 1 trilayer coating set 285, illustrated in FIG. #2, is applied to a COP syringe barrel in one embodiment.

The Set 1 trilayer coating set 285 includes as a first layer an adhesion or tie coating or layer 289 that improves adhesion of the barrier coating or layer to the COP substrate. The adhesion or tie coating or layer 289 is also believed to relieve stress on the barrier coating or layer 288, making the barrier layer less subject to damage from thermal expansion or contraction or mechanical shock. The adhesion or tie coating or layer 289 is also believed to decouple defects between the barrier coating or layer 288 and the COP substrate. This is believed to occur because any pinholes or other defects that may be formed when the adhesion or tie coating or layer 289 is applied tend not to be continued when the barrier coating or layer 288 is applied, so the pinholes or other defects in one coating do not line up with defects in the other. The adhesion or tie coating or layer 289 has some efficacy as a barrier layer, so even a defect providing a leakage path extending through the barrier coating or layer 289 is blocked by the adhesion or tie coating or layer 289.

The Set 1 trilayer coating set 285 includes as a second layer a barrier coating or layer 288 that provides a barrier to oxygen that has permeated the COP barrel wall. The barrier coating or layer 288 also is a barrier to extraction of the composition of the barrel wall 214 by the contents of the lumen 214.

The Set 1 trilayer coating set 285 includes as a third layer a pH protective coating or layer 286 that provides protection of the underlying barrier coating or layer 288 against contents of the syringe having a pH from 4 to 8, including where a surfactant is present. For a prefilled syringe that is in contact with the contents of the syringe from the time it is manufactured to the time it is used, the pH protective coating or layer 286 prevents or inhibits attack of the barrier coating or layer 288 sufficiently to maintain an effective oxygen barrier over the intended shelf life of the prefilled syringe.

Sets 5 and 6 are useful for a vial, for instance. The lubricant deposit as the coating set 285b represents a siliconized septum in which the entire surface is coated with a lubricant to aid insertion into a vial neck, so the facing surface of the closure is coated although the coating is not needed there.

The vessel wall coating set 285 represented by Set 6 is another trilayer coating set, again illustrated in FIG. 2, applied to a COP vial in one embodiment. The trilayer coating has the same layers and provides the same performance as the syringe trilayer coating of Set 1 described above.

### Tie Coating or Layer

The tie coating or layer 289 has at least two functions. One function of the tie coating or layer 289 is to improve adhesion of a barrier coating or layer 288 to a substrate, in particular a thermoplastic substrate, although a tie layer can be used to improve adhesion to a glass substrate or to another coating or layer. For example, a tie coating or layer, also referred to as an adhesion layer or coating can be applied to the substrate and the barrier layer can be applied to the adhesion layer to improve adhesion of the barrier layer or coating to the substrate.

Another function of the tie coating or layer 289 has been discovered: a tie coating or layer 289 applied under a barrier coating or layer 288 can improve the function of a pH protective coating or layer 286 applied over the barrier coating or layer 288.

The tie coating or layer 289 can be composed of, comprise, or consist essentially of SiOₓC_{y}, in which x is between 0.5 and 2.4 and y is between 0.6 and 3. Alternatively, the atomic ratio can be expressed as the formula Si_{w}OₓC_{y}, The atomic ratios of Si, O, and C in the tie coating or layer 289 are, as several options:
Si 100: O 50-150: C 90-200 (i.e. w=1, x=0.5 to 1.5, y=0.9 to 2);
Si 100: O 70-130: C 90-200 (i.e. w=1, x=0.7 to 1.3, y=0.9 to 2)
Si 100: O 80-120: C 90-150 (i.e. w=1, x=0.8 to 1.2, y=0.9 to 1.5)
Si 100: O 90-120: C 90-140 (i.e. w=1, x=0.9 to 1.2, y=0.9 to 1.4), or
Si 100: 092-107: C 116-133 (i.e. w=1, x=0.92 to 1.07, y=1.16 to 1.33)

The atomic ratio can be determined by XPS. Taking into account the H atoms, which are not measured by XPS, the tie coating or layer 289 may thus in one aspect have the formula Si_{w}OₓC_{y}H_{z} (or its equivalent SiOₓC_{y}), for example where w is 1, x is from about 0.5 to about 2.4, y is from about 0.6 to about 3, and z is from about 2 to about 9. Typically, tie coating or layer 289 would hence contain 36% to 41% carbon normalized to 100% carbon plus oxygen plus silicon.

Optionally, the tie coating or layer can be similar or identical in composition with the pH protective coating or layer 286 described elsewhere in this specification, although this is not a requirement.

The tie coating or layer 289 is contemplated in any embodiment generally to be from 5 nm to 100 nm thick, preferably from 5 to 20 nm thick, particularly if applied by chemical vapor deposition. These thicknesses are not critical. Commonly but not necessarily, the tie coating or layer 289 will be relatively thin, since its function is to change the surface properties of the substrate.

### Barrier Layer

A barrier coating or layer 288 optionally can be deposited by plasma enhanced chemical vapor deposition (PECVD) or other chemical vapor deposition processes on the vessel of a pharmaceutical package, in particular a thermoplastic package, to prevent oxygen, carbon dioxide, or other gases from entering the vessel and/or to prevent leaching of the pharmaceutical material into or through the package wall.

The barrier coating or layer for any embodiment defined in this specification (unless otherwise specified in a particular instance) is a coating or layer, optionally applied by PECVD as indicated in U.S. Pat. No. 7,985,188. The barrier layer optionally is characterized as an “SiOₓ” coating, and contains silicon, oxygen, and optionally other elements, in which x, the ratio of oxygen to silicon atoms, is from about 1.5 to about 2.9, or 1.5 to about 2.6, or about 2. These alternative definitions of x apply to any use of the term SiOₓ in this specification. The barrier coating or layer is applied, for example to the interior of a pharmaceutical package or other vessel, for example a sample collection tube, a syringe barrel, a vial, or another type of vessel.

The barrier coating 288 comprises or consists essentially of SiOₓ, wherein x is from 1.5 to 2.9, from 2 to 1000 nm thick, the barrier coating 288 of SiOₓ having an interior surface 220 facing the lumen 212 and an outer surface 222 facing the wall 214 article surface 254, the barrier coating 288 being effective to reduce the ingress of atmospheric gas into the lumen 212 compared to an uncoated vessel 250. One suitable barrier composition is one where x is 2.3, for example. For example, the barrier coating or layer such as 288 of any embodiment can be applied at a thickness of at least 2 nm, or at least 4 nm, or at least 7 nm, or at least 10 nm, or at least 20 nm, or at least 30 nm, or at least 40 nm, or at least 50 nm, or at least 100 nm, or at least 150 nm, or at least 200 nm, or at least 300 nm, or at least 400 nm, or at least 500 nm, or at least 600 nm, or at least 700 nm, or at least 800 nm, or at least 900 nm. The barrier coating or layer can be up to 1000 nm, or at most 900 nm, or at most 800 nm, or at most 700 nm, or at most 600 nm, or at most 500 nm, or at most 400 nm, or at most 300 nm, or at most 200 nm, or at most 100 nm, or at most 90 nm, or at most 80 nm, or at most 70 nm, or at most 60 nm, or at most 50 nm, or at most 40 nm, or at most 30 nm, or at most 20 nm, or at most 10 nm, or at most 5 nm thick. Ranges of 20-200 nm, optionally 20-30 nm, are contemplated. Specific thickness ranges composed of any one of the minimum thicknesses expressed above, plus any equal or greater one of the maximum thicknesses expressed above, are expressly contemplated.

The thickness of the SiOₓ or other barrier coating or layer can be measured, for example, by transmission electron microscopy (TEM), and its composition can be measured by X-ray photoelectron spectroscopy (XPS). The primer coating or layer described herein can be applied to a variety of pharmaceutical packages or other vessels made from plastic or glass, for example to plastic tubes, vials, and syringes.

A barrier coating or layer 286 of SiOₓ, in which x is between 1.5 and 2.9, is applied by plasma enhanced chemical vapor deposition (PECVD) directly or indirectly to the thermoplastic wall 214 (for example a tie coating or layer 289 can be interposed between them) so that in the filled pharmaceutical package or other vessel 210 the barrier coating or layer 286 is located between the inner or interior surface 220 of the thermoplastic wall 214 and the fluid 218.

The barrier coating or layer 286 of SiOₓ is supported by the thermoplastic wall 214. The barrier coating or layer 286 as described elsewhere in this specification, or in U.S. Pat. No. 7,985,188, can be used in any embodiment.

Certain barrier coatings or layers 286 such as SiOₓ as defined here have been found to have the characteristic of being subject to being measurably diminished in barrier improvement factor in less than six months as a result of attack by certain relatively high pH contents of the coated vessel as described elsewhere in this specification, particularly where the barrier coating or layer directly contacts the contents. This issue can be addressed using a pH protective coating or layer as discussed in this specification.

The barrier coating or layer 286 of SiOₓ also can function as a primer coating or layer 283, as discussed elsewhere in this specification.

### pH Protective Coating or Layer

It has been found that barrier layers or coatings of SiOₓ are eroded or dissolved by some fluids, for example aqueous compositions having a pH above about 5. Since coatings applied by chemical vapor deposition can be very thin—tens to hundreds of nanometers thick—even a relatively slow rate of erosion can remove or reduce the effectiveness of the barrier layer in less time than the desired shelf life of a product package. This is particularly a problem for fluid pharmaceutical compositions, since many of them have a pH of roughly 7, or more broadly in the range of 5 to 9, similar to the pH of blood and other human or animal fluids. The higher the pH of the pharmaceutical preparation, the more quickly it erodes or dissolves the SiOₓ coating. Optionally, this problem can be addressed by protecting the barrier coating or layer 288, or other pH sensitive material, with a pH protective coating or layer 286.

Optionally, the pH protective coating or layer 286 can be composed of, comprise, or consist essentially of Si_{w}OₓC_{y}H_{z} (or its equivalent SiOₓC_{y}) or Si_{w}NₓC_{y}H_{z} or its equivalent Si(NH)ₓC_{y}), each as defined previously. The atomic ratio of Si:O:C or Si:N:C can be determined by XPS (X-ray photoelectron spectroscopy). Taking into account the H atoms, the pH protective coating or layer may thus in one aspect have the formula Si_{w}OₓC_{y}H_{z}, or its equivalent SiOₓC_{y}, for example where w is 1, x is from about 0.5 to about 2.4, y is from about 1.6 to about 3, and z is from about 2 to about 9.

Typically, expressed as the formula Si_{w}OₓC_{y}, the atomic ratios of Si, O, and C are, as several options:
Si 100: O 50-150: C 90-200 (i.e. w=1, x=0.5 to 1.5, y=0.9 to 2);
Si 100: O 70-130: C 90-200 (i.e. w=1, x=0.7 to 1.3, y=0.9 to 2)
Si 100: O 80-120: C 90-150 (i.e. w=1, x=0.8 to 1.2, y=0.9 to 1.5)
Si 100: O 90-120: C 90-140 (i.e. w=1, x=0.9 to 1.2, y=0.9 to 1.4)
Si 100: O 92-107: C 116-133 (i.e. w=1, x=0.92 to 1.07, y=1.16 to 1.33), or
Si 100: O 80-130: C 90-150.

Alternatively, the pH protective coating or layer can have atomic concentrations normalized to 100% carbon, oxygen, and silicon, as determined by X-ray photoelectron spectroscopy (XPS) of less than 50% carbon and more than 25% silicon. Alternatively, the atomic concentrations are from 25 to 45% carbon, 25 to 65% silicon, and 10 to 35% oxygen. Alternatively, the atomic concentrations are from 30 to 40% carbon, 32 to 52% silicon, and 20 to 27% oxygen. Alternatively, the atomic concentrations are from 33 to 37% carbon, 37 to 47% silicon, and 22 to 26% oxygen.

The thickness of the pH protective coating or layer can be, for example:
from 10 nm to 1000 nm;
alternatively from 10 nm to 1000 nm;
alternatively from 10 nm to 900 nm;
alternatively from 10 nm to 800 nm;
alternatively from 10 nm to 700 nm;
alternatively from 10 nm to 600 nm;
alternatively from 10 nm to 500 nm;
alternatively from 10 nm to 400 nm;
alternatively from 10 nm to 300 nm;
alternatively from 10 nm to 200 nm;
alternatively from 10 nm to 100 nm;
alternatively from 10 nm to 50 nm;
alternatively from 20 nm to 1000 nm;
alternatively from 50 nm to 1000 nm;
alternatively from 10 nm to 1000 nm;
alternatively from 50 nm to 800 nm;
alternatively from 100 nm to 700 nm;
alternatively from 300 to 600 nm.

Optionally, the atomic concentration of carbon in the protective layer, normalized to 100% of carbon, oxygen, and silicon, as determined by X-ray photoelectron spectroscopy (XPS), can be greater than the atomic concentration of carbon in the atomic formula for the organosilicon precursor. For example, embodiments are contemplated in which the atomic concentration of carbon increases by from 1 to 80 atomic percent, alternatively from 10 to 70 atomic percent, alternatively from 20 to 60 atomic percent, alternatively from 30 to 50 atomic percent, alternatively from 35 to 45 atomic percent, alternatively from 37 to 41 atomic percent.

Optionally, the atomic ratio of carbon to oxygen in the pH protective coating or layer can be increased in comparison to the organosilicon precursor, and/or the atomic ratio of oxygen to silicon can be decreased in comparison to the organosilicon precursor.

Optionally, the pH protective coating or layer can have an atomic concentration of silicon, normalized to 100% of carbon, oxygen, and silicon, as determined by X-ray photoelectron spectroscopy (XPS), less than the atomic concentration of silicon in the atomic formula for the feed gas. For example, embodiments are contemplated in which the atomic concentration of silicon decreases by from 1 to 80 atomic percent, alternatively by from 10 to 70 atomic percent, alternatively by from 20 to 60 atomic percent, alternatively by from 30 to 55 atomic percent, alternatively by from 40 to 50 atomic percent, alternatively by from 42 to 46 atomic percent.

As another option, a pH protective coating or layer is contemplated in any embodiment that can be characterized by a sum formula wherein the atomic ratio C:O can be increased and/or the atomic ratio Si:O can be decreased in comparison to the sum formula of the organosilicon precursor.

The pH protective coating or layer 286 commonly is located between the barrier coating or layer 288 and the fluid 218 in the finished article. The pH protective coating or layer 286 is supported by the thermoplastic wall 214.

The pH protective coating or layer 286 optionally is effective to keep the barrier coating or layer 288 at least substantially undissolved as a result of attack by the fluid 218 for a period of at least six months.

The pH protective coating or layer can have a density between 1.25 and 1.65 g/cm³, alternatively between 1.35 and 1.55 g/cm³, alternatively between 1.4 and 1.5 g/cm³, alternatively between 1.4 and 1.5 g/cm³, alternatively between 1.44 and 1.48 g/cm³, as determined by X-ray reflectivity (XRR). Optionally, the organosilicon compound can be octamethylcyclotetrasiloxane and the pH protective coating or layer can have a density which can be higher than the density of a pH protective coating or layer made from HMDSO as the organosilicon compound under the same PECVD reaction conditions.

The pH protective coating or layer optionally can prevent or reduce the precipitation of a compound or component of a composition in contact with the pH protective coating or layer, in particular can prevent or reduce insulin precipitation or blood clotting, in comparison to the uncoated surface and/or to a barrier coated surface using HMDSO as precursor.

The pH protective coating or layer optionally can have an RMS surface roughness value (measured by AFM) of from about 5 to about 9, optionally from about 6 to about 8, optionally from about 6.4 to about 7.8. The Rₐ surface roughness value of the pH protective coating or layer, measured by AFM, can be from about 4 to about 6, optionally from about 4.6 to about 5.8. The Rₘₐₓ surface roughness value of the pH protective coating or layer, measured by AFM, can be from about 70 to about 160, optionally from about 84 to about 142, optionally from about 90 to about 130.

The interior surface of the pH protective optionally can have a contact angle (with distilled water) of from 90° to 110°, optionally from 80° to 120°, optionally from 70° to 130°, as measured by Goniometer Angle measurement of a water droplet on the pH protective surface, per ASTM D7334—08 “Standard Practice for Surface Wettability of Coatings, Substrates and Pigments by Advancing Contact Angle Measurement.”

The passivation layer or pH protective coating or layer 286 optionally shows an O-Parameter measured with attenuated total reflection (ATR) of less than 0.4, measured as:

$$O - Parameter = \frac{Intensity at 1253 cm - 1}{Maximum intensity in the range 1000 to 1100 cm - 1}$$

The O-Parameter is defined in U.S. Pat. No. 8,067,070, which claims an O-parameter value of most broadly from 0.4 to 0.9. It can be measured from physical analysis of an FTIR amplitude versus wave number plot to find the numerator and denominator of the above expression, as shown in FIG. 6, which is the same as FIG. 5 of U.S. Pat. No. 8,067,070, except annotated to show interpolation of the wave number and absorbance scales to arrive at an absorbance at 1253 cm-1 of 0.0424 and a maximum absorbance at 1000 to 1100 cm-1 of 0.08, resulting in a calculated O-parameter of 0.53. The O-Parameter can also be measured from digital wave number versus absorbance data.

U.S. Pat. No. 8,067,070 asserts that the claimed O-parameter range provides a superior pH protective coating or layer, relying on experiments only with HMDSO and HMDSN, which are both non-cyclic siloxanes. Surprisingly, it has been found by the present inventors that if the PECVD precursor is a cyclic siloxane, for example OMCTS, O-parameters outside the ranges claimed in U.S. Pat. No. 8,067,070, using OMCTS, provide even better results than are obtained in U.S. Pat. No. 8,067,070 with HMDSO.

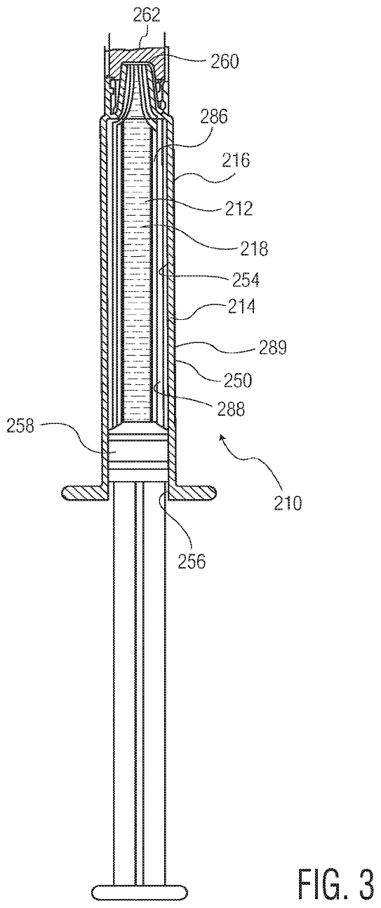

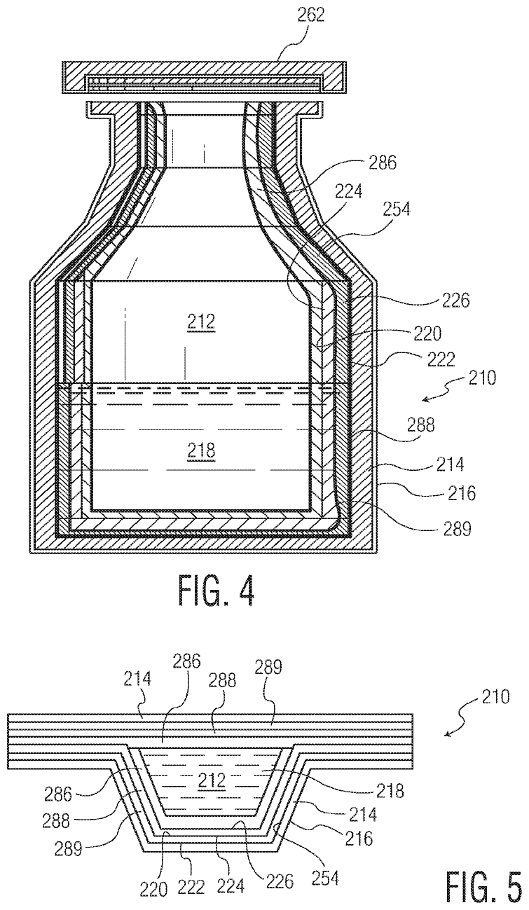

Alternatively in the embodiment of FIGS. 1-5, the O-parameter has a value of from 0.1 to 0.39, or from 0.15 to 0.37, or from 0.17 to 0.35.

Even another aspect of the invention is a composite material as just described, exemplified in FIGS. 1-5, wherein the passivation layer shows an N-Parameter measured with attenuated total reflection (ATR) of less than 0.7, measured as:

$$N - Parameter = \frac{Intensity at 840 cm - 1}{Intensity at 799 cm - 1} .$$

The N-Parameter is also described in U.S. Pat. No. 8,067,070, and is measured analogously to the O-Parameter except that intensities at two specific wave numbers are used—neither of these wave numbers is a range. U.S. Pat. No. 8,067,070 claims a passivation layer with an N-Parameter of 0.7 to 1.6. Again, the present inventors have made better coatings employing a pH protective coating or layer 286 having an N-Parameter lower than 0.7, as described above. Alternatively, the N-parameter has a value of at least 0.3, or from 0.4 to 0.6, or at least 0.53.

The rate of erosion, dissolution, or leaching (different names for related concepts) of the pH protective coating or layer 286, if directly contacted by the fluid 218, is less than the rate of erosion of the barrier coating or layer 288, if directly contacted by the fluid 218.

The thickness of the pH protective coating or layer is contemplated in any embodiment to be from 50-500 nm, with a preferred range of 100-200 nm.

The pH protective coating or layer 286 is effective to isolate the fluid 218 from the barrier coating or layer 288, at least for sufficient time to allow the barrier coating to act as a barrier during the shelf life of the pharmaceutical package or other vessel 210.

The inventors have further found that certain pH protective coatings or layers of SiOₓC_{y} or Si(NH)ₓC_{y} formed from polysiloxane precursors, which pH protective coatings or layers have a substantial organic component, do not erode quickly when exposed to fluids, and in fact erode or dissolve more slowly when the fluids have higher pHs within the range of 5 to 9. For example, at pH 8, the dissolution rate of a pH protective coating or layer made from the precursor octamethylcyclotetrasiloxane, or OMCTS, is quite slow. These pH protective coatings or layers of SiOₓC_{y} or Si(NH)ₓC_{y} can therefore be used to cover a barrier layer of SiOₓ, retaining the benefits of the barrier layer by protecting it from the fluid in the pharmaceutical package. The protective layer is applied over at least a portion of the SiOₓ layer to protect the SiOₓ layer from contents stored in a vessel, where the contents otherwise would be in contact with the SiOₓ layer.

Although the present invention does not depend upon the accuracy of the following theory, it is further believed that effective pH protective coatings or layers for avoiding erosion can be made from siloxanes and silazanes as described in this disclosure. SiOₓC_{y} or Si(NH)ₓCy coatings deposited from cyclic siloxane or linear silazane precursors, for example octamethylcyclotetrasiloxane (OMCTS), are believed to include intact cyclic siloxane rings and longer series of repeating units of the precursor structure. These coatings are believed to be nanoporous but structured and hydrophobic, and these properties are believed to contribute to their success as pH protective coatings or layers, and also protective coatings or layers. This is shown, for example, in U.S. Pat. No. 7,901,783.

SiOₓC_{y} or Si(NH)ₓC_{y} coatings also can be deposited from linear siloxane or linear silazane precursors, for example hexamethyldisiloxane (HMDSO) or tetramethyldisiloxane (TMDSO).

Optionally an FTIR absorbance spectrum of the pH protective coating or layer 286 of any embodiment has a ratio greater than 0.75 between the maximum amplitude of the Si—O—Si symmetrical stretch peak normally located between about 1000 and 1040 cm-1, and the maximum amplitude of the Si—O—Si as symmetric stretch peak normally located between about 1060 and about 1100 cm-1. Alternatively in any embodiment, this ratio can be at least 0.8, or at least 0.9, or at least 1.0, or at least 1.1, or at least 1.2. Alternatively in any embodiment, this ratio can be at most 1.7, or at most 1.6, or at most 1.5, or at most 1.4, or at most 1.3. Any minimum ratio stated here can be combined with any maximum ratio stated here, as an alternative embodiment of the invention of FIGS. 1-5.

Optionally, in any embodiment the pH protective coating or layer 286, in the absence of the medicament, has a non-oily appearance. This appearance has been observed in some instances to distinguish an effective pH protective coating or layer from a lubricity layer, which in some instances has been observed to have an oily (i.e. shiny) appearance.

Optionally, for the pH protective coating or layer 286 in any embodiment, the silicon dissolution rate by a 50 mM potassium phosphate buffer diluted in water for injection, adjusted to pH 8 with concentrated nitric acid, and containing 0.2 wt. % polysorbate-80 surfactant, (measured in the absence of the medicament, to avoid changing the dissolution reagent), at 40° C., is less than 170 ppb/day. (Polysorbate-80 is a common ingredient of pharmaceutical preparations, available for example as Tween8-80 from Uniqema Americas LLC, Wilmington Delaware.) Optionally, for the pH protective coating or layer 286 in any embodiment, the silicon dissolution rate is less than 160 ppb/day, or less than 140 ppb/day, or less than 120 ppb/day, or less than 100 ppb/day, or less than 90 ppb/day, or less than 80 ppb/day. Optionally, in any embodiment of FIGS. 24-26 the silicon dissolution rate is more than 10 ppb/day, or more than 20 ppb/day, or more than 30 ppb/day, or more than 40 ppb/day, or more than 50 ppb/day, or more than 60 ppb/day. Any minimum rate stated here can be combined with any maximum rate stated here for the pH protective coating or layer 286 in any embodiment.

Optionally, for the pH protective coating or layer 286 in any embodiment the total silicon content of the pH protective coating or layer and barrier coating, upon dissolution into a test composition with a pH of 8 from the vessel, is less than 66 ppm, or less than 60 ppm, or less than 50 ppm, or less than 40 ppm, or less than 30 ppm, or less than 20 ppm.

The inventors offer the following theory of operation of the pH protective coating or layer described here. The invention is not limited by the accuracy of this theory or to the embodiments predictable by use of this theory.

The dissolution rate of the SiOₓ barrier layer is believed to be dependent on SiO bonding within the layer. Oxygen bonding sites (silanols) are believed to increase the dissolution rate.

It is believed that the OMCTS-based pH protective coating or layer bonds with the silanol sites on the SiOₓ barrier layer to “heal” or passivate the SiOₓ surface and thus dramatically reduces the dissolution rate. In this hypothesis, the thickness of the OMCTS layer is not the primary means of protection—the primary means is passivation of the SiOₓ surface. It is contemplated in any embodiment that a pH protective coating or layer as described in this specification can be improved by increasing the crosslink density of the pH protective coating or layer.

### Hydrophobic Layer

The protective or lubricity coating or layer of Si_{w}OₓC_{y} or its equivalent SiOₓC_{y} also can have utility as a hydrophobic layer, independent of whether it also functions as a pH protective coating or layer Suitable hydrophobic coatings or layers and their application, properties, and use are described in U.S. Pat. No. 7,985,188. Dual functional protective/hydrophobic coatings or layers having the properties of both types of coatings or layers can be provided for any embodiment of the present invention.

An embodiment can be carried out under conditions effective to form a hydrophobic pH protective coating or layer on the substrate. Optionally, the hydrophobic characteristics of the pH protective coating or layer can be set by setting the ratio of the 02 to the organosilicon precursor in the gaseous reactant, and/or by setting the electric power used for generating the plasma. Optionally, the pH protective coating or layer can have a lower wetting tension than the uncoated surface, optionally a wetting tension of from 20 to 72 dyne/cm, optionally from 30 to 60 dynes/cm, optionally from 30 to 40 dynes/cm, optionally 34 dyne/cm. Optionally, the pH protective coating or layer can be more hydrophobic than the uncoated surface.

Use of a coating or layer according to any described embodiment is contemplated in any embodiment as (i) a lubricity coating having a lower frictional resistance than the uncoated surface; and/or (ii) a pH protective coating or layer preventing dissolution of the barrier coating in contact with a fluid, and/or (iii) a hydrophobic layer that is more hydrophobic than the uncoated surface.

### Lubricity Layer

A “lubricity layer” or any similar term is generally defined as a coating that reduces the frictional resistance of the coated surface, relative to the uncoated surface. If the coated object is a syringe (or syringe part, e.g. syringe barrel) or any other item generally containing a plunger or movable part in sliding contact with the coated surface, the frictional resistance has two main aspects—breakout force and plunger sliding force.

The plunger sliding force test is a specialized test of the coefficient of sliding friction of the plunger within a syringe, accounting for the fact that the normal force associated with a coefficient of sliding friction as usually measured on a flat surface is addressed by standardizing the fit between the plunger or other sliding element and the tube or other vessel within which it slides. The parallel force associated with a coefficient of sliding friction as usually measured is comparable to the plunger sliding force measured as described in this specification. Plunger sliding force can be measured, for example, as provided in the ISO 7886-1:1993 test.

The plunger sliding force test can also be adapted to measure other types of frictional resistance, for example the friction retaining a stopper within a tube, by suitable variations on the apparatus and procedure. In one embodiment, the plunger can be replaced by a closure and the withdrawing force to remove or insert the closure can be measured as the counterpart of plunger sliding force.

Also or instead of the plunger sliding force, the breakout force can be measured. The breakout force is the force required to start a stationary plunger moving within a syringe barrel, or the comparable force required to unseat a seated, stationary closure and begin its movement. The breakout force is measured by applying a force to the plunger that starts at zero or a low value and increases until the plunger begins moving. The breakout force tends to increase with storage of a syringe, after the prefilled syringe plunger has pushed away the intervening lubricant or adhered to the barrel due to decomposition of the lubricant between the plunger and the barrel. The breakout force is the force needed to overcome “sticktion,” an industry term for the adhesion between the plunger and barrel that needs to be overcome to break out the plunger and allow it to begin moving.

Some utilities of coating a vessel in whole or in part with a lubricity layer, such as selectively at surfaces contacted in sliding relation to other parts, is to ease the insertion or removal of a stopper or passage of a sliding element such as a piston in a syringe or a stopper in a sample tube. The vessel can be made of glass or a polymer material such as polyester, for example polyethylene terephthalate (PET), a cyclic olefin copolymer (COC), a cyclic block co-polymer (CBC), an olefin such as polypropylene, or other materials. Examples of cyclic block co-polymers include, for example, those in the VIVION™ family, such as VIVION™ 0510 or VIVION™ 0510HF, manufactured by USI Corporation (Taiwan). Applying a lubricity layer by PECVD can avoid or reduce the need to coat the vessel wall or closure with a sprayed, dipped, or otherwise applied organosilicon or other lubricant that commonly is applied in a far larger quantity than would be deposited by a PECVD process.

The power (in Watts) used for PECVD also has an influence on the coating properties. Typically, an increase of the power will increase the barrier properties of the coating, and a decrease of the power will increase the lubricity of the coating. E.g., for a coating on the inner wall of syringe barrel having a volume of about 3 ml, a power of less than 30 W will lead to a coating which is predominantly a barrier layer, while a power of more than 30 W will lead to a coating which is predominantly a lubricity layer.

A further parameter determining the coating properties is the ratio of O₂ (or another oxidizing agent) to the precursor (e.g. organosilicon precursor) in the gaseous reactant used for generating the plasma. Typically, an increase of the O₂ ratio in the gaseous reactant will increase the barrier properties of the coating, and a decrease of the O₂ ratio will increase the lubricity of the coating.

If a lubricity layer is desired, then 02 is optionally present in a volume-volume ratio to the gaseous reactant of from 0:1 to 5:1, optionally from 0:1 to 1:1, even optionally from 0:1 to 0.5:1 or even from 0:1 to 0.1:1. Most advantageously, essentially no oxygen is present in the gaseous reactant. Thus, the gaseous reactant will in some embodiments comprise less than 1 vol % O₂, for example less than 0.5 vol % O₂, and optionally is O₂-free.

A process is contemplated for applying a lubricity layer characterized as defined in the Definition Section on a substrate, for example the interior of the barrel of a syringe, comprising applying one of the described precursors on or in the vicinity of a substrate at a thickness of 1 to 5000 nm, optionally 10 to 1000 nm, optionally 10-200 nm, optionally 20 to 100 nm thick and crosslinking or polymerizing (or both) the coating, optionally in a PECVD process, to provide a lubricated surface.

A coating of SiwOxCy as defined in the Definition Section optionally can be very thin, having a thickness of at least 4 nm, or at least 7 nm, or at least 10 nm, or at least 20 nm, or at least 30 nm, or at least 40 nm, or at least 50 nm, or at least 100 nm, or at least 150 nm, or at least 200 nm, or at least 300 nm, or at least 400 nm, or at least 500 nm, or at least 600 nm, or at least 700 nm, or at least 800 nm, or at least 900 nm. The coating can be up to 1000 nm, or at most 900 nm, or at most 800 nm, or at most 700 nm, or at most 600 nm, or at most 500 nm, or at most 400 nm, or at most 300 nm, or at most 200 nm, or at most 100 nm, or at most 90 nm, or at most 80 nm, or at most 70 nm, or at most 60 nm, or at most 50 nm, or at most 40 nm, or at most 30 nm, or at most 20 nm, or at most 10 nm, or at most 5 nm thick. Specific thickness ranges composed of any one of the minimum thicknesses expressed above, plus any equal or greater one of the maximum thicknesses expressed above, are expressly contemplated.

A lubricity layer, characterized as defined in the Definition Section, can be applied as a subsequent coating after applying any combination of layers described herein to the interior surface 88 of the vessel 80 to provide a lubricity layer.

Optionally, after the lubricity layer is applied, it can be post-cured after the PECVD process. Radiation curing approaches, including UV-initiated (free radial or cationic), electron-beam (E-beam), and thermal as described in Development Of Novel Cycloaliphatic Siloxanes For Thermal And UV-Curable Applications (Ruby Chakraborty Dissertation, can 2008) be utilized.

A lubricity layer, characterized as defined in the Definition Section, is particularly contemplated for the internal surface of a syringe barrel as further described below. A lubricated internal surface of a syringe barrel can reduce the plunger sliding force needed to advance a plunger in the barrel during operation of a syringe, or the breakout force to start a plunger moving after the prefilled syringe plunger has pushed away the intervening lubricant or adhered to the barrel, for example due to decomposition of the lubricant between the plunger and the barrel.

Thus, the coating 90 can comprise a barrier layer of SiOx or a trilayer and a lubricity layer, characterized as defined in the Definition Section. The lubricity layer of Si_{w}OₓC_{y}H_{z} can be deposited between the layer of SiOₓ or the trilayer and the vessel lumen.

Another embodiment is a lubricity layer, characterized as defined in the Definition Section, on the inner wall of a syringe barrel. The coating is produced from a PECVD process using the following materials and conditions. A cyclic precursor is optionally employed, selected from a monocyclic siloxane, a polycyclic siloxane, or a combination of two or more of these, as defined elsewhere in this specification for lubricity layers. One example of a suitable cyclic precursor comprises octamethylcyclotetrasiloxane (OMCTS), optionally mixed with other precursor materials in any proportion. Optionally, the cyclic precursor consists essentially of octamethylcyclotetrasiloxane (OMCTS), meaning that other precursors can be present in amounts which do not change the basic and novel properties of the resulting lubricity layer, i.e. its reduction of the plunger sliding force or breakout force of the coated surface.

Optionally, at least essentially no oxygen, as defined in the Definition Section is added to the process.

A sufficient plasma generation power input, for example any power level successfully used in one or more working examples of this specification or described in the specification, is provided to induce coating formation.

The materials and conditions employed are effective to reduce the syringe plunger sliding force or breakout force moving through the syringe barrel at least 25 percent, alternatively at least 45 percent, alternatively at least 60 percent, alternatively greater than 60 percent, relative to an uncoated syringe barrel. Ranges of plunger sliding force or breakout force reduction of from 20 to 95 percent, alternatively from 30 to 80 percent, alternatively from 40 to 75 percent, alternatively from 60 to 70 percent, are contemplated.

Another embodiment is a syringe including a plunger, a syringe barrel, and a lubricity layer, characterized as defined in the Definition Section. The syringe barrel includes an interior surface receiving the plunger for sliding. The lubricity layer is disposed on the interior surface of the syringe barrel. The lubricity layer is less than 1000 nm thick and effective to reduce the breakout force or the plunger sliding force necessary to move the plunger within the barrel. Reducing the plunger sliding force is alternatively expressed as reducing the coefficient of sliding friction of the plunger within the barrel or reducing the plunger force; these terms are regarded as having the same meaning in this specification.

Additional details regarding embodiments of lubricity layers and the formation of embodiments of lubricity layers via PECVD can be found in U.S. patent application Ser. No. 16/504,636, which was published as US Patent Application Publication No. 2019/0328299 A1, the entirety of which is incorporated by reference herein.

### PECVD Process

Some conditions used for production of pH Protective Layers or Lubricity Layers are shown in Table 1.

TABLE 1
OMCTS-BASED pH PROTECTIVE OR LUBRICITY LAYER MADE WITH CARRIER GAS

| | | | | | | Carrier | |
|---|---|---|---|---|---|---|---|
| | | | pH | protective | | Gas | pH |
| | pH | | protective | OMCTS | protective | (Ar) | protective |
| | protective | PH | coating or | Flow | O2 Flow | Flow | coating or |
| | coating or | protective | layer Time | Rate | Rate | Rate | layer Power |
| Example | layer Type | Monomer | (sec) | (sccm) | (sccm) | (sccm) | (Watts) |
| 1 | Uncoated | n/a | n/a | n/a | n/a | n/a | n/a |
| (Control) | COC | | | | | | |
| 2 | Silicon oil | n/a | n/a | n/a | n/a | n/a | n/a |
| (Industry | on COC | | | | | | |
| Standard) | | | | | | | |
| 3 | L3 lubricity | OMCTS | 10 sec | 3 | 0 | 65 | 6 |
| (without | coating or | | | | | | |
| Oxygen) | layer over | | | | | | |
| | SiOₓ on | | | | | | |
| | COC | | | | | | |
| 4 | L2 pH | OMCTS | 10 sec | 3 | 1 | 65 | 6 |
| (with | protective | | | | | | |
| Oxygen) | coating or | | | | | | |
| | layer over | | | | | | |
| | SiOₓ on | | | | | | |
| | COC | | | | | | |

The PECVD trilayer coating described in this specification can be applied, for example, as follows for a 1 to 5 mL vessel. Two specific examples are 1 mL thermoplastic resin syringe and a 5 mL thermoplastic resin drug vial. Larger or smaller vessels will call for adjustments in parameters that a person of ordinary skill can carry out in view of the teaching of this specification.

The apparatus used is the PECVD apparatus with rotating quadrupole magnets as described generally in this specification.

The general coating parameter ranges, with preferred ranges in parentheses, for a trilayer coating for a 1 mL syringe barrel are shown in the PECVD Trilayer Process General Parameters Tables (1 mL syringe and 5 mL vial).

PECVD Trilayer Process General Parameters Table (1 mL syringe)

| Parameter | Units | Tie | Barrier | pH Protective |
|---|---|---|---|---|
| Power | W | 40-90 | 140 | 40-90 |
| | | (60-80) | | (60-80) |
| TMDSO Flow | sccm | 1-10 | None | 1-10 |
| | | (3-5) | | (3-5) |
| HMDSO Flow | sccm | None | 1.56 | None |
| O₂ Flow | sccm | 0.5-5 | 20 | 0.5-5 |
| | | (1.5-2.5) | | (1.5-2.5) |
| Argon Flow | sccm | 40-120 | 0 | 40-120 |
| | | (70-90) | | (70-90) |
| Ramp Time | seconds | None | None | None |
| Deposition | seconds | 0.1-10 | 20 | 0.1-40 |
| Time | | (1-3) | | (15-25) |
| Tube | Torr | 0.01-10 | 0.59 | 0.01-10 |
| Pressure | | (0.1-1.5) | | (0.1-1.5) |

PECVD Trilayer Process General Parameters Table (5 mL vial)

| Parameter | Units | Adhesion | Barrier | Protection |
|---|---|---|---|---|
| Power | W | 40-90 | 140 | 40-90 |
| | | (60-80) | | (60-80) |
| TMDSO Flow | sccm | 1-10 | None | 1-10 |
| | | (3-5) | | (3-5) |
| HMDSO Flow | sccm | None | 1.56 | None |
| O₂ Flow | sccm | 0.5-5 | 20 | 0.5-5 |
| | | (1.5-2.5) | | (1.5-2.5) |
| Argon Flow | sccm | 40-120 | 0 | 40-120 |
| | | (70-90) | | (70-90) |
| Ramp Time | seconds | None | None | None |
| Deposition | seconds | 0.1-10 | 20 | 0.1-40 |
| Time | | (1-3) | | (15-25) |
| Tube | Torr | 0.01-10 | 0.59 | 0.01-10 |
| Pressure | | (0.1-1.5) | | (0.1-1.5) |

Examples of specific coating parameters that have been used for a 1 mL syringe and 5 mL vial are shown in the PECVD Trilayer Process Specific Parameters Tables (1 mL syringe and 5 mL vial):

PECVD Trilayer Process Specific Parameters Table (1 mL syringe)

| Parameter | Units | Tie | Barrier | Protection |
|---|---|---|---|---|
| Power | W | 70 | 140 | 70 |
| TMDSO Flow | sccm | 4 | None | 4 |
| HMDSO Flow | sccm | None | 1.56 | None |
| O₂ Flow | sccm | 2 | 20 | 2 |
| Argon Flow | sccm | 80 | 0 | 80 |
| Ramp Time | seconds | None | None | None |
| Deposition | seconds | 2.5 | 20 | 10 |
| Time | | | | |
| Tube | Torr | 1 | 0.59 | 1 |
| Pressure | | | | |

PECVD Trilayer Process Specific Parameters Table (5 mL vial)

| Parameter | Units | Adhesion | Barrier | Protection |
|---|---|---|---|---|
| Power | W | 20 | 40 | 20 |
| TMDSO Flow | sccm | 2 | 0 | 2 |
| HMDSO Flow | sccm | 0 | 3 | 0 |
| O₂ Flow | sccm | 1 | 50 | 1 |
| Argon Flow | sccm | 20 | 0 | 20 |
| Ramp Time | seconds | 0 | 2 | 2 |
| Deposition | seconds | 2.5 | 10 | 10 |
| Time | | | | |
| Tube | Torr | 0.85 | 1.29 | 0.85 |
| Pressure | | | | |

The O-parameter and N-parameter values for the pH protective coating or layer applied to the 1 mL syringe as described above are 0.34 and 0.55, respectively. The O-parameter and N-parameter values for the pH protective coating or layer applied to the 5 mL vial are 0.24 and 0.63, respectively.

### Protocol for Total Silicon Measurement

This protocol is used to determine the total amount of silicon coatings present on the entire vessel wall. A supply of 0.1 N potassium hydroxide (KOH) aqueous solution is prepared, taking care to avoid contact between the solution or ingredients and glass. The water used is purified water, 18 Mn quality. A Perkin Elmer Optima Model 7300DV ICP-OES instrument is used for the measurement except as otherwise indicated.

Each device (vial, syringe, tube, or the like) to be tested and its cap and crimp (in the case of a vial) or other closure are weighed empty to 0.001 g, then filled completely with the KOH solution (with no headspace), capped, crimped, and reweighed to 0.001 g. In a digestion step, each vial is placed in an autoclave oven (liquid cycle) at 121° C. for 1 hour. The digestion step is carried out to quantitatively remove the silicon coatings from the vessel wall into the KOH solution. After this digestion step, the vials are removed from the autoclave oven and allowed to cool to room temperature. The contents of the vials are transferred into ICP tubes. The total Si concentration is run on each solution by ICP/OES following the operating procedure for the ICP/OES.

The total Si concentration is reported as parts per billion of Si in the KOH solution. This concentration represents the total amount of silicon coatings that were on the vessel wall before the digestion step was used to remove it.

The total Si concentration can also be determined for fewer than all the silicon layers on the vessel, as when an SiOx barrier layer is applied, an SiOxCy second layer (for example, a lubricity layer or a primer coating or layer) is then applied, and it is desired to know the total silicon concentration of just the SiOxCy layer. This determination is made by preparing two sets of vessels, one set to which only the SiOx layer is applied and the other set to which the same SiOx layer is applied, followed by the SiOxCy layer or other layers of interest. The total Si concentration for each set of vessels is determined in the same manner as described above. The difference between the two Si concentrations is the total Si concentration of the SiOₓC_{y} second layer.

### Protocol for Measuring Dissolved Silicon In a Vessel

In some of the working examples, the amount of silicon dissolved from the wall of the vessel by a test solution is determined, in parts per billion (ppb), for example to evaluate the dissolution rate of the test solution. This determination of dissolved silicon is made by storing the test solution in a vessel provided with an SiOₓ and/or SiOₓC_{y} coating or layer under test conditions, then removing a sample of the solution from the vessel and testing the Si concentration of the sample. The test is done in the same manner as the Protocol for Total Silicon Measurement, except that the digestion step of that protocol is replaced by storage of the test solution in the vessel as described in this protocol. The total Si concentration is reported as parts per billion of Si in the test solution

### Protocol for Determining Average Dissolution Rate

The average dissolution rates reported in the working examples are determined as follows. A series of test vessels having a known total silicon measurement are filled with the desired test solution analogous to the manner of filling the vials with the KOH solution in the Protocol for Total Silicon Measurement. (The test solution can be a physiologically inactive test solution as employed in the present working examples or a physiologically active pharmaceutical preparation intended to be stored in the vessels to form a pharmaceutical package). The test solution is stored in respective vessels for several different amounts of time, then analyzed for the Si concentration in parts per billion in the test solution for each storage time. The respective storage times and Si concentrations are then plotted. The plots are studied to find a series of substantially linear points having the steepest slope.

The plot of dissolution amount (ppb Si) versus days decreases in slope with time, even though it does not appear that the Si layer has been fully digested by the test solution.

For the PC194 test data in Table 10 below, linear plots of dissolution versus time data are prepared by using a least squares linear regression program to find a linear plot corresponding to the first five data points of each of the experimental plots. The slope of each linear plot is then determined and reported as representing the average dissolution rate applicable to the test, measured in parts per billion of Si dissolved in the test solution per unit of time.

### Protocol for Determining Calculated Shelf Life

The calculated shelf life values reported in the working examples are determined by extrapolation of the total silicon measurements and average dissolution rates, respectively determined as described in the Protocol for Total Silicon Measurement and the Protocol for Determining Average Dissolution Rate. The assumption is made that under the indicated storage conditions the SiOₓC_{y} primer coating or layer will be removed at the average dissolution rate until the coating is entirely removed. Thus, the total silicon measurement for the vessel, divided by the dissolution rate, gives the period of time required for the test solution to totally dissolve the SiOₓC_{y} coating. This period of time is reported as the calculated shelf life. Unlike commercial shelf life calculations, no safety factor is calculated. Instead, the calculated shelf life is the calculated time to failure.

It should be understood that because the plot of ppb Si versus hours decreases in slope with time, an extrapolation from relatively short measurement times to relatively long calculated shelf lives is believed to be a “worst case” test that tends to underestimate the calculated shelf life actually obtainable.

### Prophetic Example 1

The present inventors have developed a series of experiments to demonstrate the effect of metal ion exchange as a mechanism for polysorbate degradation. The experiment will focus on vials among the following:
1. Uncoated COP vials
2. Trilayer coated COP vials
3. Type 1 borosilicate glass vial
4. Type 1 borosilicate glass vial having silicon oil on the interior surface
5. COP vial having silicon oil on the interior surface

Each vial is to be filled with a simulated drug composed of a pH buffered aqueous solution containing polysorbate at a concentration about ten times higher of a typical concentration of polysorbate in aqueous drug solutions. These solutions may include:
1. A pH6 solution using a citrate or phosphate buffer and polysorbate 80 at 0.2 wt. %
2. A pH6 solution using a citrate or phosphate buffer and polysorbate 20 at 0.2 wt. %
3. A pH5 solution using a citrate or phosphate buffer and polysorbate 80 at 0.2 wt. %
4. A pH5 solution using a citrate or phosphate buffer and polysorbate 20 at 0.2 wt. %
5. A pH4 solution using a citrate or phosphate buffer and polysorbate 80 at 0.2 wt. %
6. A pH4 solution using a citrate or phosphate buffer and polysorbate 20 at 0.2 wt. %
7. A pH3 solution using a citrate or phosphate buffer and polysorbate 80 at 0.2 wt. %
8. A pH3 solution using a citrate or phosphate buffer and polysorbate 20 at 0.2 wt. %

The vials are filled with the solution and stored at ambient temperature (25° C.) and accelerated temperature (40° C.). The vials are stored for six months and tested at regular intervals:
1. T=0
2. T=1 month
3. T=3 months
4. T=6 months
   Ten replicates are tested per time interval. The vials are tested for:
1. Polysorbate degradation using liquid chromatography-mass spectrometry (LC-MS)
2. Metal ions in the simulated solution using inductively coupled plasma-optical emission spectrometry (ICP-OES)

The amount of polysorbate—here either PS80 or PS20—in the solution is tested using LC-MS. In particular, the concentration of polysorbate—here either PS80 or PS20—may be tested using an Agilent quadrupole time-of flight (Q-ToF) LC-MS system. Solutions are appropriately diluted, e.g. with a 40/60 water/acetonitrile diluent, and then analyzed using positive electroscopy ionization. For quantification of PS80 or PS20, a collection of representative ions, e.g. ten ions having representative mass-to-charge ratios (m/z), are extracted and integrated. Separation may be performed with a C18 column and a water/acetonitrile gradient with 0.1% formic acid and 0.04% ammonium formate. Representative calibration solutions are also prepared and analyzed.

The amount/concentration of metal ions in the simulated solution is tested using ICP-OES. In particular, a Perkin Elmer Optima Model 7300DV ICP-OES instrument is used for the measurement except as otherwise indicated. The contents of the vials are transferred into ICP tubes. The total metal ion concentration is run on each solution by ICP/OES following the operating procedure for the ICP/OES instrument. The total concentration of metal ions in the solution is reported as parts per billion. The respective storage times and metal ion concentrations are then plotted.

It is expected that the solutions stored in the trilayer coated COP vials will show significantly less degradation of polysorbate over time than the solutions stored in the borosilicate glass vials. It is also expected that the solutions stored in the trilayer coated COP vials will contain significantly fewer metal ions over time than the solutions stored in the borosilicate glass vial. Indeed, it is expected that the solutions stored in the trilayer coated COP vials will contain zero or substantially zero metal ions at all test intervals.

What is claimed:

1. A method of reducing the degradation of an aqueous drug product, comprising
providing a vessel having a lumen defined at least in part by a wall, the wall having an interior surface facing the lumen, the interior surface comprising a coating that is free or substantially free of metal ions; and
storing an aqueous drug product, optionally comprising a polysorbate excipient, in the lumen of the vessel, such that the aqueous drug product is in contact with the coating;
wherein the vessel is effective to reduce
(i) degradation of an active agent of the aqueous drug product;
(ii) degradation of an excipient of the aqueous drug product, optionally polysorbate;
(iii) the formation of protein aggregates in the aqueous drug product;
(iv) the formation of protein aggregate particles in the aqueous drug product;
(v) the presence of metal leachables; or
(vi) any combination of (i) through (v)
relative to the same aqueous drug product stored under identical conditions in an otherwise identical vessel but in which the interior surface of the vessel rather than the coating is in contact with the drug composition.

2. Use of a vessel having a lumen defined at least in part by a wall, the wall having an interior surface facing the lumen, and the interior surface comprising a coating that is free or substantially free of metal ions, to reduce
(i) degradation of an active agent;
(ii) degradation of an excipient, optionally polysorbate;
(iii) the formation of protein aggregates;
(iv) the formation of protein aggregate particles;
(v) the presence of metal leachables; or
(vi) any combination of (i) through (v)
in a drug-containing solution stored within the lumen and in contact with the coating.

3. A method of reducing the polysorbate concentration of a drug-containing solution within a drug primary package, comprising:
providing a vessel having a lumen defined at least in part by a wall, the wall having an interior surface facing the lumen, the interior surface comprising a coating that is free or substantially free of metal ions;
storing a drug-containing solution in the lumen of the vessel, such that the drug-containing solution is in contact with the coating, wherein the drug-containing solution comprises a polysorbate stabilizer and the coating is effective to reduce degradation of the polysorbate stabilizer in the solution during storage; and
reducing a concentration of polysorbate in a formulation of the drug-containing solution relative to that of the same drug-containing solution formulated for the same vessel but without the coating and/or that of the same drug-containing solution formulated for storage in a glass vessel.

4. Use of a vessel having a lumen defined at least in part by a wall, the wall having an interior surface facing the lumen, and the interior surface comprising a coating that is effective to reduce degradation of the polysorbate stabilizer in a solution within the lumen during storage, to reduce the concentration of polysorbate in a formulation of the drug-containing solution stored within the lumen of the vessel relative to that of the same drug-containing solution formulated for storage in the same vessel but without the coating and/or that of the same drug-containing solution formulated for storage in a glass vessel.

5. The method/use of any one preceding claim, wherein the coating is configured so that leaching of metal ions into the aqueous drug product or drug-containing solution from the vessel wall is avoided, optionally wherein the coating is configured so that leaching of metal ions into the aqueous drug product or drug-containing solution from the vessel wall is avoided for aqueous drug products or drug-containing solutions having pHs between 2 and 9.

6. The method/use of any one preceding claim, wherein the coating is configured to withstand dissolution by the aqueous drug product or drug-containing solution, optionally wherein the coating is configured to withstand dissolution by aqueous drug products or drug-containing solutions having pHs between 2 and 9.

7. The method/use of any one preceding claim, wherein the silicon dissolution rate of the coating by a 50 mM potassium phosphate buffer diluted in water, adjusted to pH 8 with concentrated nitric acid, and containing 0.2 wt. % polysorbate-80 surfactant, at 40° C., is less than 170 ppb/day, optionally less than 160 ppb/day, optionally less than 140 ppb/day, optionally less than 120 ppb/day, optionally less than 100 ppb/day, optionally less than 90 ppb/day, optionally less than 80 ppb/day.

8. The method/use of any one preceding claim, in which the aqueous drug product or drug-containing solution has a pH between 5 and 9, optionally between 5 and 8, optionally between 5 and 7, optionally wherein the aqueous drug product or drug-containing solution has a pH of 5, optionally wherein the aqueous drug product or drug-containing solution has a pH of 6, optionally wherein the aqueous drug product or drug-containing solution has a pH of 7, optionally wherein the aqueous drug product or drug-containing solution has a pH of 8, optionally wherein the aqueous drug product or drug-containing solution has a pH of 9.

9. The method/use of any one preceding claim, wherein the aqueous drug product or drug-containing solution removes the coating at a rate of 1 nm or less of coating thickness per 44 hours of contact, alternatively a rate of 1 nm or less of coating thickness per 88 hours of contact, alternatively a rate of 1 nm or less of coating thickness per 350 hours of contact.

10. The method/use of any one preceding claim, wherein the coating is further configured to serve as a lubricity coating.

11. The method/use of any one preceding claim, wherein the coating is an organosilicon, optionally an organosiloxane, optionally an organosiloxane having the chemical formula Si_{w}OₓC_{y}, optionally in which w is 1, x is from about 0.5 to 2.4, and y is from about 0.6 to about 3.

12. The method/use of any one preceding claim, wherein the Si_{w}OₓC_{y} coating is formed by PECVD using a feed gas that comprises siloxane precursor; optionally a linear siloxane precursor or a monocyclic siloxane precursor or a polycyclic siloxane precursor, optionally a linear siloxane precursor, optionally a monocyclic siloxane precursor.

13. The method/use of any one preceding claim, wherein the coating is an organo silazane, optionally an organosilazane having the chemical formula Si_{w}NₓC_{y}, optionally in which w is 1, x is from about 0.5 to 2.4, and y is from about 0.6 to about 3.

14. The method/use of any one preceding claim, wherein the coating comprises or consists essentially of SiOxCy or SiNxCy, wherein x is from about 0.5 to about 2.4 and y is from about 0.6 to about 3.

15. The method/use of any one preceding claim, wherein the coating is an amorphous carbon coating, optionally an amorphous carbon coating applied by PECVD.

16. The method/use of any one preceding claim, wherein the coating is a silicon carbide coating or silicon nitride coating, optionally a silicon carbide coating or silicon nitride coating applied by PECVD.

17. The method/use of any one preceding claim, in which the coating has a thickness of 10 to 1000 nm, optionally 10 to 500 nm, optionally 10 to 200 nm, optionally 20 to 100 nm.

18. The method/use of any one preceding claim, further comprising an oxygen barrier layer, the oxygen barrier layer being effective to reduce the ingress of atmospheric gas into the lumen compared to the same vessel without an oxygen barrier layer.

19. The method/use of any one preceding claim, wherein the oxygen barrier layer comprises SiOx, wherein x is from 1.5 to 2.9.

20. The method/use of any one preceding claim, in which the oxygen barrier layer comprises or consists essentially of a plurality of atomic monolayers, optionally wherein the oxygen barrier layer is applied by atomic layer deposition, optionally plasma-assisted atomic layer deposition.

21. The method/use of any one preceding claim, in which the oxygen barrier layer is applied by PECVD.

22. The method/use of any one preceding claim, further comprising a pH protective coating or layer, the pH protective coating or layer being effective to increase the calculated shelf life of the vessel.

23. The method/use of any one preceding claim, wherein the pH protective coating or layer is positioned between the oxygen barrier coating or layer and the lumen.

24. The method/use of any one preceding claim, wherein the pH protective coating or layer comprises SiOxCy or SiNxCy, wherein x is from about 0.5 to about 2.4 and y is from about 0.6 to about 3.

25. The method/use of any one preceding claim, further comprising an adhesion coating or layer.

26. The method/use of any one preceding claim, wherein the adhesion coating or layer is positioned between the oxygen barrier coating or layer and the interior surface of the wall.

27. The method/use of any one preceding claim, wherein the adhesion coating or layer comprises SiOxCy or SiNxCy, wherein x is from about 0.5 to about 2.4 and y is from about 0.6 to about 3.

28. The method/use of any one preceding claim, further comprising a water vapor barrier coating or layer, the water vapor barrier coating or layer being effective to reduce the ingress of water vapor into the lumen compared to the same vessel without the water vapor barrier coating or layer.

29. The method/use of any one preceding claim, wherein the water vapor barrier coating or layer comprises or consists essentially of a metal oxide, optionally Al₂O₃.

30. The method/use of any one preceding claim, wherein the coating comprises:
a tie coating or layer comprising SiOₓC_{y} or SiNₓC_{y} wherein x is from about 0.5 to about 2.4 and y is from about 0.6 to about 3, the tie coating or layer having an interior surface facing the lumen and an outer surface facing the wall interior surface;
a barrier coating or layer comprising SiOₓ, wherein x is from 1.5 to 2.9, the barrier coating or layer of SiOₓ having an interior surface facing the lumen and an outer surface facing the interior surface of the tie coating or layer, the barrier coating or layer being effective to reduce the ingress of atmospheric gas into the lumen compared to a vessel without a barrier coating or layer;
a drug-contacting coating or layer comprising SiOxCy or SiNxCy wherein x is from about 0.5 to about 2.4 and y is from about 0.6 to about 3, the drug-contacting coating or layer having an interior surface facing the lumen and an outer surface facing the interior surface of the barrier coating or layer.

31. The method/use of any one preceding claim, wherein the drug-contacting layer also serves as a pH protective layer.

32. The method/use of any one preceding claim, wherein the drug-contacting layer also serves as a lubricity layer.

33. The method/use of any one preceding claim, wherein the drug solution has a pH between about 3 and about 14.

34. The method/use of any one preceding claim, wherein the drug product has a pH between 5 and 9 and the calculated shelf life of the container is more than six months at a storage temperature of 4° C.

35. The method/use of any one preceding claim, in which the vessel wall is a thermoplastic material, optionally a thermoplastic material having residual catalyst material.

36. The method/use of any one preceding claim, in which the vessel wall is a plastic selected from the group consisting of COP, COC, PET, PETG, polypropylene, a polyamide, polystyrene, polycarbonate, TRITAN™, a cyclic block copolymer (CBC) resin, polyethylene, HDPE, LDPE, a thermoplastic olefinic polymer, or any combination thereof.

37. The method/use of any one preceding claim, in which the vessel wall is a cyclic olefin polymer (COP) resin, a cyclic olefin copolymer (COC) resin, or a cyclic block co-polymer (CBC) resin.

38. The method/use of any one preceding claim, in which the vessel wall is selected from the group consisting of VIVION™ 0510, VIVION™ 0510HF, and VIVION™ 1325; optionally the group consisting of VIVION™ 0510 and VIVION™ 0510HF; optionally VIVION™ 0510; optionally VIVION™ 0510HF.

39. The method/use of any one preceding claim, wherein the vessel wall comprises borosilicate glass.

40. The method/use of any one preceding claim, wherein the metal ions include one or more of the following: Na, K, Al, Mg, Ca, Ba, Fe, Mn, Zn, and Li.

41. The method/use of any one preceding claim, wherein the vessel is a syringe or a vial, optionally a syringe, optionally a vial.

42. The method/use of any one preceding claim, wherein the vessel lumen has a volume of 10 mL or less, optionally a volume of 5 mL or less, optionally a volume of 2 mL or less.

43. The method/use of any one preceding claim, wherein the storing takes place for at least 1 month, optionally at least two months, optionally at least 3 months, optionally at least 4 months, optionally at least 5 months, optionally at least 6 months, optionally between 1 month and 12 months, optionally between 1 month and 6 months, optionally 1 month, optionally 3 months, optionally 6 months.

44. The method/use of any one preceding claim, wherein the polysorbate is selected from the group consisting of polysorbate 80 and polysorbate 20, optionally wherein the polysorbate is polysorbate 80, optionally wherein the polysorbate is polysorbate 20.

45. The method/use of any preceding claim, in which the protein aggregates and/or particles are a product of polysorbate degradation.

46. The method/use of any one preceding claim, wherein the active agent comprises a protein-based drug; optionally a monoclonal antibody; optionally a monoclonal antibody selected from the group consisting of mAb, mAb-A, and mAb-B; optionally a drug selected from the group consisting of IgG1 mAb, IgG4 mAb, Fab, scFv, nanobodies, nanobody monomers, nanobody dimers, nanobody trimers, nanobody tetramers, nanobody pentamers, nanobody dimer-PEG, nanobody dimer-HSA, Bispecific T-cell engagers (BiTE), and Dual-Affinity ReTargeting antibodies (DART).

47. The method/use of any one preceding claim, wherein the aqueous drug product or drug-containing solution comprises a vaccine, optionally a vaccine comprising a mineral adjuvant.

48. The method/use of any one preceding claim, wherein the aqueous drug product or drug-containing solution comprises a radioisotope.

49. The method/use of any one preceding claim, wherein the aqueous drug product or drug-containing solution is a biologic drug product comprising a peptide active agent.

50. The method/use of any one preceding claim, wherein the aqueous drug product or drug-containing solution comprises polysorbate, and wherein the reduction in polysorbate degradation achieved by the coating allows for a reduction in an initial concentration of polysorbate in the aqueous drug product or drug-containing solution relative to a formulation of the same aqueous drug product or drug-containing solution for storage in the same vessel but without the coating.

51. The method/use of any one preceding claim, in which the aqueous drug product or drug-containing solution comprises a material or formulation selected from the group consisting of:
BIOLOGIC DRUGS
abatacept; abciximab; abobotulinumtoxinA; adalimumab; adalimumab-adaz; adalimumab-adbm; adalimumab-afzb; adalimumab-atto; adalimumab-bwwd; ado-trastuzumab emtansine; aflibercept; agalsidase beta; albiglutide; albumin chromated CR-51 serum; aldesleukin; alefacept; alemtuzumab; alglucosidase alfa; alirocumab; alteplase; anakinra; aprotinin; asfotas alfa; asparaginase; asparaginase Erwinia chrysanthemi; atezolizumab; avelumab; basiliximab; becaplermin; belatacept; belimumab; benralizumab; beractant; bevacizumab; bevacizumab-awwb; bevacizumab-bvzr; beziotoxumab; blinatumomab; brentuximab vedotin; brodalumab; brolucizumab-dbll; burosumab-twza; calaspargase pegol-mknl; calfactant; canakinumab; caplacizumab-yhdp; capromab pendetide; cemiplimab-rwlc; cenegermin-bkbj; cerliponase alfa; certolizumab pegol; cetuximab; choriogonadotropin alfa; chorionic gonadotropin; chymopapain; collagenase; collagenase clostridium histolyticum; corticorelin ovine triflutate; crizanlizumab-tmca; daclizumab; daratumumab; daratumumab and hyaluronidase-fihj; darbepoetin alpha; denileukin diftitox; denosumab; desirudin; dinutuximab; domase alfa; drotrecogin alfa; dulaglutide; dupilumab; durvalumab; ecallantide; eculizumab; efalizumab; elapegademase-lvlr; elosulfase alfa; elotuzumab; emapalumab-lzsg; emicizumab-kxwh; enfortumab vedotin-ejfv; epoetin alfa; epoetin alfa-epbx; erenumab-aooe; etanercept; etanercept-szzs; etanercept-ykro; evolocumab; fam-trastuzumab deruxetecan-nxki; fibrinolysin and desoxyribonuclease combined [bovine], with chioramphenicol; filgrastim; filgrastim-aafi; filgrastim-sndz; follitropin alfa; follitropin beta; fremanezumab-vfrm; galcanezumab-gnlm; galsulfase; gemtuzumab ozogamicin; glucarpidase; golimumab; guselkumab; hyaluronidase; hyaluronidase human; ibalizumab-uiyk; ibritumomab tiuxetan; idarucizumab; idursulfase; imiglucerase; incobotulinumtoxinA; inebilizumab-cdon; infliximab; infliximab-abda; infliximab-axxq; infliximab-dyyb; infliximab-qbtx; inotuzumab ozogamicin; insulin aspart; insulin aspart protamine and insulin aspart; insulin degludec; insulin degludec and insulin aspart; insulin degludec and liraglutide; insulin detemir; insulin glargine; insulin glargine and lixisenatide; insulin glulisine; insulin human; insulin isophane human; insulin isophane human and insulin human; insulin lispro; insulin lispro protamine and insulin lispro; insulin lispro-aabc; interferon alfa-2a; interferon alfa-2b; interferon alfacon-1; interferon alfa-n3 (human leukocyte derived); interferon beta-1a; interferon beta-1b; interferon gamma-1b; ipilimumab; isatuximab-irfc; ixekizumab; lanadelumab-flyo; laronidase; lixisenatide; luspatercept-aamt; mecasermin; mecasermin rinfabate; menotropins; mepolizumab; methoxy polyethylene glycol-epoetin beta; metreleptin; mogamulizumab-kpkc; moxetumomab pasudotox-tdfk; muromanab-CD3; natalizumab; necitumumab; nivolumab; nofetumomab; obiltoxaximab; obinutuzumab; ocrelizumab; ocriplasmin; ofatumumab; olaratumab; omalizumab; onabotulinumtoxinA; oprelvekin; palifermin; palivizumab; pancrelipase; panitumumab; parathyroid hormone; pegademase bovine; pegaspargase; pegfilgrastim; pegfilgrastim-apgf; pegfilgrastim-bmez; pegfilgrastim-cbqv; pegfilgrastim-jmdb; peginterferon alfa-2a; peginterferon alfa-2a and ribavirin; peginterferon alfa-2b; peginterferon alfa-2b and ribavirin; peginterferon beta-1a; pegloticase; pegvaliase-pqpz; pegvisomant; pembrolizumab; pertuzumab; polatuzumab vedotin-piiq; poractant alfa; prabotulinumtoxinA-xvfs; radiolabeled albumin technetium Tc-99m albumin colloid kit; ramucirumab; ranibizumab; rasburicase; ravulizumab-cwvz; raxibacumab; reslizumab; reteplase; rilonacept; rimabotulinumtoxinB; risankizumab-rzaa; rituximab; rituximab and hyaluronidase human; rituximab-abbs; rituximab-pvvr; romiplostim; romosozumab-aqqg; sacituzumab govitecan-hziy; sacrosidase; sargramostim; sarilumab; sebelipase alfa; secukinumab; siltuximab; somatropin; tagraxofusp-erzs; taliglucerase alfa; tbo-filgrastim; technetium 99m tc fanolesomab; tenecteplase; teprotumumab-trbw; tesamorelin acetate; thyrotropin alfa; tildrakizumab-asmn; tocilizumab; tositumomab and iodine I-131 tositumomab; trastuzumab; trastuzumab and hyaluronidase-oysk; trastuzumab-anns; trastuzumab-dkst; trastuzumab-dttb; trastuzumab-pkrb; trastuzumab-qyyp; urofollitropin; urokinase; ustekinumab; vedolizumab; velaglucerase alfa; vestronidase alfa-vjbk; Ziv-Aflibercept; Amjevita (adalimumab-atto); Dupixent (dupilumab); Fulphila (pegfilgrastim-jmdb); Ilaris (canakinumab); Ixifi (infliximab-qbtx); Lyumjev (insulin lispro-aabc); Nyvepria (pegfilgrastim-apgf); Ogivri (trastuzumab-dkst); Semglee (insulin glargine); Uplizna (inebilizumab-cdon); A.P.L. (chorionic gonadotropin); Abrilada (adalimumab-afzb); Accretropin (somatropin); Actemra (tocilizumab); Acthrel (corticorelin ovine triflutate); Actimmune (interferon gamma-1b); Activase (alteplase); Adagen (pegademase bovine); Adakveo (crizanlizumab-tmca); Adcetris (brentuximab vedotin); Adlyxin (lixisenatide); Admelog (insulin lispro); Afrezza (insulin human); Aimovig (erenumab-aooe); Ajovy (fremanezumab-vfrm); Aldurazyme (laronidase); Alferon N Injection (interferon alfa-n3 (human leukocyte derived)); Amevive (alefacept); Amphadase (hyaluronidase); Anthim (obiltoxaximab); Apidra (insulin glulisine); Aranesp (darbepoetin alpha); Arcalyst (rilonacept); Arzerra (ofatumumab); Asparlas (calaspargase pegol-mknl); Avastin (bevacizumab); Avonex (interferon beta-1a); Avsola (infliximab-axxq); Basaglar (insulin glargine); Bavencio (avelumab); Benlysta (belimumab); Beovu (brolucizumab-dbll); Besponsa (inotuzumab ozogamicin); Betaseron (interferon beta-1b); Bexxar (tositumomab and iodine I-131 tositumomab); Blincyto (blinatumomab); Botox (onabotulinumtoxinA); Botox Cosmetic (onabotulinumtoxinA); Bravelle (urofollitropin); Brineura (cerliponase alfa); Cablivi (caplacizumab-yhdp); Campath (alemtuzumab); Cathflo Activase (alteplase); Cerezyme (imiglucerase); Chorionic Gonadotropin (chorionic gonadotropin); Chromalbin (albumin chromated CR-51 serum); Chymodiactin (chymopapain); Cimzia (certolizumab pegol); Cinqair (reslizumab); Cosentyx (secukinumab); Cotazym (pancrelipase); Creon (pancrelipase); Crysvita (burosumab-twza); Curosurf (poractant alfa); Cyltezo (adalimumab-adbm); Cyramza (ramucirumab); Darzalex (daratumumab); Darzalex Faspro (daratumumab and hyaluronidase-fihj); Draximage MAA (kit for the preparation of technetium Tc-99m albumin aggregated); Dysport (abobotulinumtoxinA); Egrifta (tesamorelin acetate); Egrifta SV (tesamorelin acetate); Elaprase (idursulfase); Elase-chloromycetin (fibrinolysin and desoxyribonuclease combined [bovine], with chloramphenicol); Elelyso (taliglucerase alfa); Elitek (rasburicase); Elspar (asparaginase); Elzonris (tagraxofusp-erzs); Emgality (galcanezumab-gnlm); Empliciti (elotuzumab); Enbrel (etanercept); Enbrel Mini (etanercept); Enhertu (fam-trastuzumab deruxetecan-nxki); Entyvio (vedolizumab); Epogen/Procrit (epoetin alfa); Erbitux (cetuximab); Erelzi (etanercept-szzs); Erelzi Sensoready (etanercept-szzs); Erwinaze (asparaginase Erwinia chrysanthemi); Eticovo (etanercept-ykro); Evenity (romosozumab-aqqg); Extavia (interferon beta-1b); Eylea (aflibercept); Fabrazyme (agalsidase beta); Fasenra (benralizumab); Fiasp (insulin aspart); Follistim (follitropin beta); Follistim AQ (follitropin beta); Follistim AQ Cartridge (follitropin beta); Gamifant (emapalumab-lzsg); Gazyva (obinutuzumab); Genotropin (somatropin); Gonal-f (follitropin alfa); Gonal-f RFF (follitropin alfa); Gonal-f RFF RediJect (follitropin alfa); Granix (tbo-filgrastim); Hadlima (adalimumab-bwwd); Hemlibra (emicizumab-kxwh); Herceptin (trastuzumab); Herceptin Hylecta (trastuzumab and hyaluronidase-oysk); Herzuma (trastuzumab-pkrb); Humalog (insulin lispro); Humalog Mix 50/50 (insulin lispro protamine and insulin lispro); Humalog Mix 75/25 (insulin lispro protamine and insulin lispro); Humatrope (somatropin); Humegon (menotropins); Humira (adalimumab); Humulin 70/30 (insulin isophane human and insulin human); Humulin N (insulin isophane human); Humulin R U-100 (insulin human); Humulin R U-500 (insulin human); Hydase (hyaluronidase); Hylenex recombinant (hyaluronidase human); Hyrimoz (adalimumab-adaz); Ilumya (tildrakizumab-asmn); Imfinzi (durvalumab); Increlex (mecasermin); Infasurf (calfactant); Infergen (interferon alfacon-1); Inflectra (infliximab-dyyb); Intron A (interferon alfa-2b); Iplex (mecasermin rinfabate); lprivask (desirudin); Jeanatope (kit for iodinated 1-125 albumin); Jetrea (ocriplasmin); Jeuveau (prabotulinumtoxinA-xvfs); Kadcyla (ado-trastuzumab emtansine); Kalbitor (ecallantide); Kanjinti (trastuzumab-anns); Kanuma (sebelipase alfa); Kepivance (palifermin); Kevzara (sarilumab); Keytruda (pembrolizumab); Kineret (anakinra); Kinlytic (urokinase); Krystexxa (pegloticase); Lantus (insulin glargine); Lartruvo (olaratumab); Lemtrada (alemtuzumab); Leukine (sargramostim); Levemir (insulin detemir); Libtayo (cemiplimab-rwlc); Lucentis (ranibizumab); Lumizyme (alglucosidase alfa); Lumoxiti (moxetumomab pasudotox-tdfk); Macrotec (kit for the preparation of technetium Tc-99m albumin aggregated); Megatope (kit for iodinated 1-131 albumin); Menopur (menotropins); Mepsevii (vestronidase alfa-vjbk); Microlite (radiolabeled albumin technetium Tc-99m albumin colloid kit); Mircera (methoxy polyethylene glycol-epoetin beta); Mvasi (bevacizumab-awwb); Myalept (metreleptin); Mylotarg (gemtuzumab ozogamicin); Myobloc (rimabotulinumtoxinB); Myozyme (alglucosidase alfa); Myxredlin (insulin human); N/A (raxibacumab); Naglazyme (galsulfase); Natpara (parathyroid hormone); Neulasta (pegfilgrastim); Neulasta Onpro (pegfilgrastim); Neumega (oprelvekin); Neupogen (filgrastim); NeutroSpec (technetium 99m tc fanolesomab); Nivestym (filgrastim-aafi); Norditropin (somatropin); Novarel (chorionic gonadotropin); Novolin 70/30 (insulin isophane human and insulin human); Novolin N (insulin isophane human); Novolin R (insulin human); Novolog (insulin aspart); Novolog Mix 50/50 (insulin aspart protamine and insulin aspart); Novolog Mix 70/30 (insulin aspart protamine and insulin aspart); Nplate (romiplostim); Nucala (mepolizumab); Nulojix (belatacept); Nutropin (somatropin); Nutropin AQ (somatropin); Ocrevus (ocrelizumab); Omnitrope (somatropin); Oncaspar (pegaspargase); Ontak (denileukin diftitox); Ontruzant (trastuzumab-dttb); Opdivo (nivolumab); Orencia (abatacept); Orthoclone OKT3 (muromanab-CD3); Ovidrel (choriogonadotropin alfa); Oxervate (cenegermin-bkbj); Padcev (enfortumab vedotin-ejfv); Palynziq (pegvaliase-pqpz); Pancreaze (pancrelipase); Pegasys (peginterferon alfa-2a); Pegasys Copegus Combination Pack (peginterferon alfa-2a and ribavirin); Pegintron (peginterferon alfa-2b); Pegintron/Rebetol Combo Pack (peginterferon alfa-2b and ribavirin); Pergonal (menotropins); Perjeta (pertuzumab); Pertzye (pancrelipase); Plegridy (peginterferon beta-1a); Polivy (polatuzumab vedotin-piiq); Portrazza (necitumumab); Poteligeo (mogamulizumab-kpkc); Praluent (alirocumab); Praxbind (idarucizumab); Pregnyl (chorionic gonadotropin); Procrit (epoetin alfa); Proleukin (aldesleukin); Prolia (denosumab); ProstaScint (capromab pendetide); Pulmolite (kit for the preparation of technetium Tc-99m albumin aggregated); Pulmotech MAA (kit for the preparation of technetium Tc-99m albumin aggregated); Pulmozyme (dornase alfa); Raptiva (efalizumab); Rebif (interferon beta-1a); Reblozyl (luspatercept-aamt); Regranex (becaplermin); Remicade (infliximab); Renflexis (infliximab-abda); Reopro (abciximab); Repatha (evolocumab); Repronex (menotropins); Retacrit (epoetin alfa-epbx); Retavase (reteplase); Revcovi (elapegademase-lvlr); Rituxan (rituximab); Rituxan Hycela (rituximab and hyaluronidase human); Roferon-A (interferon alfa-2a); Ruxience (rituximab-pvvr); Ryzodeg 70/30 (insulin degludec and insulin aspart); Saizen (somatropin); Santyl (collagenase); Sarclisa (isatuximab-irfc); Serostim (somatropin); Siliq (brodalumab); Simponi (golimumab); Simponi Aria (golimumab); Simulect (basiliximab); Skyrizi (risankizumab-rzaa); Soliqua 100/33 (insulin glargine and lixisenatide); Soliris (eculizumab); Somavert (pegvisomant); Stelara (ustekinumab); Strensiq (asfotas alfa); Sucraid (sacrosidase); Survanta (beractant); Sylvant (siltuximab); Synagis (palivizumab); Takhzyro (lanadelumab-flyo); Taltz (ixekizumab); Tanzeum (albiglutide); Tecentriq (atezolizumab); Tepezza (teprotumumab-trbw); Thyrogen (thyrotropin alfa); TNKase (tenecteplase); Toujeo (insulin glargine); Trasylol (aprotinin); Trazimera (trastuzumab-qyyp); Tremfya (guselkumab); Tresiba (insulin degludec); Trodelvy (sacituzumab govitecan-hziy); Trogarzo (ibalizumab-uiyk); Trulicity (dulaglutide); Truxima (rituximab-abbs); Tysabri (natalizumab); Udenyca (pegfilgrastim-cbqv); Ultomiris (ravulizumab-cwvz); Unituxin (dinutuximab); Vectibix (panitumumab); Verluma (nofetumomab); Vimizim (elosulfase alfa); Viokace (pancrelipase); Vitrase (hyaluronidase); Voraxaze (glucarpidase); VPRIV (velaglucerase alfa); Xeomin (incobotulinumtoxinA); Xgeva (denosumab); Xiaflex (collagenase clostridium histolyticum); Xigris (drotrecogin alfa); Xolair (omalizumab); Xultophy 100/3.6 (insulin degludec and liraglutide); Yervoy (ipilimumab); Zaltrap (Ziv-Aflibercept); Zarxio (filgrastim-sndz); Zenapax (daclizumab); Zenpep (pancrelipase); Zevalin (ibritumomab tiuxetan); Ziextenzo (pegfilgrastim-bmez); Zinbryta (daclizumab); Zinplava (bezlotoxumab); Zirabev (bevacizumab-bvzr); Zomacton (somatropin); Zorbtive/Serostim (somatropin);
INHALATION ANESTHETICS
Aliflurane; Chloroform; Cyclopropane; Desflurane (Suprane); Diethyl Ether; Enflurane (Ethrane); Ethyl Chloride; Ethylene; Halothane (Fluothane); Isoflurane (Forane, Isoflo); Isopropenyl vinyl ether; Methoxyflurane; methoxyflurane; Methoxypropane; Nitrous Oxide; Roflurane; Sevoflurane (Sevorane, Ultane, Sevoflo); Teflurane; Trichloroethylene; Vinyl Ether; Xenon
INJECTABLE DRUGS
Ablavar (Gadofosveset Trisodium Injection); Abarelix Depot; Abobotulinumtoxin A Injection (Dysport); ABT-263; ABT-869; ABX-EFG; Accretropin (Somatropin Injection); Acetadote (Acetylcysteine Injection); Acetazolamide Injection (Acetazolamide Injection); Acetylcysteine Injection (Acetadote); Actemra (Tocilizumab Injection); Acthrel (Corticorelin Ovine Triflutate for Injection); Actummune; Activase; Acyclovir for Injection (Zovirax Injection); Adacel; Adalimumab; Adenoscan (Adenosine Injection); Adenosine Injection (Adenoscan); Adrenaclick; AdreView (lobenguane I 123 Injection for Intravenous Use); Afluria; Ak-Fluor (Fluorescein Injection); Aldurazyme (Laronidase); Alglucerase Injection (Ceredase); Alkeran Injection (Melphalan Hcl Injection); Allopurinol Sodium for Injection (Aloprim); Aloprim (Allopurinol Sodium for Injection); Alprostadil; Alsuma (Sumatriptan Injection); ALTU-238; Amino Acid Injections; Aminosyn; Apidra; Apremilast; Alprostadil Dual Chamber System for Injection (Caverject Impulse); AMG 009; AMG 076; AMG 102; AMG 108; AMG 114; AMG 162; AMG 220; AMG 221; AMG 222; AMG 223; AMG 317; AMG 379; AMG 386; AMG 403; AMG 477; AMG 479; AMG 517; AMG 531; AMG 557; AMG 623; AMG 655; AMG 706; AMG 714; AMG 745; AMG 785; AMG 811; AMG 827; AMG 837; AMG 853; AMG 951; Amiodarone HCl Injection (Amiodarone HCl Injection); Amobarbital Sodium Injection (Amytal Sodium); Amytal Sodium (Amobarbital Sodium Injection); Anakinra; Anti-Abeta; Anti-Beta7; Anti-Beta20; Anti-CD4; Anti-CD20; Anti-CD40; Anti-IFNalpha; Anti-IL13; Anti-OX40L; Anti-oxLDS; Anti-NGF; Anti-NRP1; Arixtra; Amphadase (Hyaluronidase Inj); Ammonul (Sodium Phenylacetate and Sodium Benzoate Injection); Anaprox; Anzemet Injection (Dolasetron Mesylate Injection); Apidra (Insulin Glulisine [rDNA origin] Inj); Apomab; Aranesp (darbepoetin alfa); Argatroban (Argatroban Injection); Arginine Hydrochloride Injection (R-Gene 10); Aristocort; Aristospan; Arsenic Trioxide Injection (Trisenox); Articane HCl and Epinephrine Injection (Septocaine); Arzerra (Ofatumumab Injection); Asclera (Polidocanol Injection); Ataluren; Ataluren-DMD; Atenolol Inj (Tenormin I.V. Injection); Atracurium Besylate Injection (Atracurium Besylate Injection); Avastin; Azactam Injection (Aztreonam Injection); Azithromycin (Zithromax Injection); Aztreonam Injection (Azactam Injection); Baclofen Injection (Lioresal Intrathecal); Bacteriostatic Water (Bacteriostatic Water for Injection); Baclofen Injection (Lioresal Intrathecal); Bal in Oil Ampules (Dimercarprol Injection); BayHepB; BayTet; Benadryl; Bendamustine Hydrochloride Injection (Treanda); Benztropine Mesylate Injection (Cogentin); Betamethasone Injectable Suspension (Celestone Soluspan); Bexxar; Bicillin C-R 900/300 (Penicillin G Benzathine and Penicillin G Procaine Injection); Blenoxane (Bleomycin Sulfate Injection); Bleomycin Sulfate Injection (Blenoxane); Boniva Injection (Ibandronate Sodium Injection); Botox Cosmetic (OnabotulinumtoxinA for Injection); BR3-FC; Bravelle (Urofollitropin Injection); Bretylium (Bretylium Tosylate Injection); Brevital Sodium (Methohexital Sodium for Injection); Brethine; Briobacept; BTT-1023; Bupivacaine HCl; Byetta; Ca-DTPA (Pentetate Calcium Trisodium Inj); Cabazitaxel Injection (Jevtana); Caffeine Alkaloid (Caffeine and Sodium Benzoate Injection); Calcijex Injection (Calcitrol); Calcitrol (Calcijex Injection); Calcium Chloride (Calcium Chloride Injection 10%); Calcium Disodium Versenate (Edetate Calcium Disodium Injection); Campath (Altemtuzumab); Camptosar Injection (Irinotecan Hydrochloride); Canakinumab Injection (Ilaris); Capastat Sulfate (Capreomycin for Injection); Capreomycin for Injection (Capastat Sulfate); Cardiolite (Prep kit for Technetium Tc99 Sestamibi for Injection); Carticel; Cathflo; Cefazolin and Dextrose for Injection (Cefazolin Injection); Cefepime Hydrochloride; Cefotaxime; Ceftriaxone; Cerezyme; Camitor Injection; Caverject; Celestone Soluspan; Celsior; Cerebyx (Fosphenytoin Sodium Injection); Ceredase (Alglucerase Injection); Ceretec (Technetium Tc99m Exametazime Injection); Certolizumab; CF-101; Chloramphenicol Sodium Succinate (Chloramphenicol Sodium Succinate Injection); Chloramphenicol Sodium Succinate Injection (Chloramphenicol Sodium Succinate); Cholestagel (Colesevelam HCL); Choriogonadotropin Alfa Injection (Ovidrel); Cimzia; Cisplatin (Cisplatin Injection); Clolar (Clofarabine Injection); Clomiphine Citrate; Clonidine Injection (Duraclon); Cogentin (Benztropine Mesylate Injection); Colistimethate Injection (Coly-Mycin M); Coly-Mycin M (Colistimethate Injection); Compath; Conivaptan Hcl Injection (Vaprisol); Conjugated Estrogens for Injection (Premarin Injection); Copaxone; Corticorelin Ovine Triflutate for Injection (Acthrel); Corvert (lbutilide Fumarate Injection); Cubicin (Daptomycin Injection); CF-101; Cyanokit (Hydroxocobalamin for Injection); Cytarabine Liposome Injection (DepoCyt); Cyanocobalamin; Cytovene (ganciclovir); D.H.E. 45; Dacetuzumab; Dacogen (Decitabine Injection); Dalteparin; Dantrium IV (Dantrolene Sodium for Injection); Dantrolene Sodium for Injection (Dantrium IV); Daptomycin Injection (Cubicin); Darbepoietin Alfa; DDAVP Injection (Desmopressin Acetate Injection); Decavax; Decitabine Injection (Dacogen); Dehydrated Alcohol (Dehydrated Alcohol Injection); Denosumab Injection (Prolia); Delatestryl; Delestrogen; Delteparin Sodium; Depacon (Valproate Sodium Injection); Depo Medrol (Methylprednisolone Acetate Injectable Suspension); DepoCyt (Cytarabine Liposome Injection); DepoDur (Morphine Sulfate XR Liposome Injection); Desmopressin Acetate Injection (DDAVP Injection); Depo-Estradiol; Depo-Provera 104 mg/ml; Depo-Provera 150 mg/ml; Depo-Testosterone; Dexrazoxane for Injection, Intravenous Infusion Only (Totect); Dextrose/Electrolytes; Dextrose and Sodium Chloride Inj (Dextrose 5% in 0.9% Sodium Chloride); Dextrose; Diazepam Injection (Diazepam Injection); Digoxin Injection (Lanoxin Injection); Dilaudid-HP (Hydromorphone Hydrochloride Injection); Dimercarprol Injection (Bal in Oil Ampules); Diphenhydramine Injection (Benadryl Injection); Dipyridamole Injection (Dipyridamole Injection); DMOAD; Docetaxel for Injection (Taxotere); Dolasetron Mesylate Injection (Anzemet Injection); Doribax (Doripenem for Injection); Doripenem for Injection (Doribax); Doxercalciferol Injection (Hectorol Injection); Doxil (Doxorubicin Hcl Liposome Injection); Doxorubicin Hcl Liposome Injection (Doxil); Duraclon (Clonidine Injection); Duramorph (Morphine Injection); Dysport (Abobotulinumtoxin A Injection); Ecallantide Injection (Kalbitor); EC-Naprosyn (naproxen); Edetate Calcium Disodium Injection (Calcium Disodium Versenate); Edex (Alprostadil for Injection); Engerix; Edrophonium Injection (Enlon); Eliglustat Tartate; Eloxatin (Oxaliplatin Injection); Emend Injection (Fosaprepitant Dimeglumine Injection); Enalaprilat Injection (Enalaprilat Injection); Enlon (Edrophonium Injection); Enoxaparin Sodium Injection (Lovenox); Eovist (Gadoxetate Disodium Injection); Enbrel (etanercept); Enoxaparin; Epicel; Epinepherine; Epipen; Epipen Jr.; Epratuzumab; Erbitux; Ertapenem Injection (Invanz); Erythropoieten; Essential Amino Acid Injection (Nephramine); Estradiol Cypionate; Estradiol Valerate; Etanercept; Exenatide Injection (Byetta); Evlotra; Fabrazyme (Adalsidase beta); Famotidine Injection; FDG (Fludeoxyglucose F 18 Injection); Feraheme (Ferumoxytol Injection); Feridex I.V. (Ferumoxides Injectable Solution); Fertinex; Ferumoxides Injectable Solution (Feridex I.V.); Ferumoxytol Injection (Feraheme); Flagyl Injection (Metronidazole Injection); Fluarix; Fludara (Fludarabine Phosphate); Fludeoxyglucose F 18 Injection (FDG); Fluorescein Injection (Ak-Fluor); Follistim AQ Cartridge (Follitropin Beta Injection); Follitropin Alfa Injection (Gonal-f RFF); Follitropin Beta Injection (Follistim AQ Cartridge); Folotyn (Pralatrexate Solution for Intravenous Injection); Fondaparinux; Forteo (Teriparatide (rDNA origin) Injection); Fostamatinib; Fosaprepitant Dimeglumine Injection (Emend Injection); Foscarnet Sodium Injection (Foscavir); Foscavir (Foscarnet Sodium Injection); Fosphenytoin Sodium Injection (Cerebyx); Fospropofol Disodium Injection (Lusedra); Fragmin; Fuzeon (enfuvirtide); GA101; Gadobenate Dimeglumine Injection (Multihance); Gadofosveset Trisodium Injection (Ablavar); Gadoteridol Injection Solution (ProHance); Gadoversetamide Injection (OptiMARK); Gadoxetate Disodium Injection (Eovist); Ganirelix (Ganirelix Acetate Injection); Gardasil; GC1008; GDFD; Gemtuzumab Ozogamicin for Injection (Mylotarg); Genotropin; Gentamicin Injection; GENZ-112638; Golimumab Injection (Simponi Injection); Gonal-f RFF (Follitropin Alfa Injection); Granisetron Hydrochloride (Kytril Injection); Gentamicin Sulfate; Glatiramer Acetate; Glucagen; Glucagon; HAE1; Haldol (Haloperidol Injection); Havrix; Hectorol Injection (Doxercalciferol Injection); Hedgehog Pathway Inhibitor; Heparin; Herceptin; hG-CSF; Humalog; Human Growth Hormone; Humatrope; HuMax; Humegon; Humira; Humulin; Ibandronate Sodium Injection (Boniva Injection); Ibuprofen Lysine Injection (NeoProfen); Ibutilide Fumarate Injection (Corvert); Idamycin PFS (Idarubicin Hydrochloride Injection); Idarubicin Hydrochloride Injection (Idamycin PFS); Ilaris (Canakinumab Injection); Imipenem and Cilastatin for Injection (Primaxin I.V.); Imitrex; Incobotulinumtoxin A for Injection (Xeomin); Increlex (Mecasermin [rDNA origin] Injection); Indocin IV (Indomethacin Inj); Indomethacin Inj (Indocin IV); Infanrix; Innohep; Insulin; Insulin Aspart [rDNA origin] Inj (NovoLog); Insulin Glargine [rDNA origin] Injection (Lantus); Insulin Glulisine [rDNA origin] Inj (Apidra); Interferon alfa-2b, Recombinant for Injection (Intron A); Intron A (Interferon alfa-2b, Recombinant for Injection); Invanz (Ertapenem Injection); Invega Sustenna (Paliperidone Palmitate Extended-Release Injectable Suspension); Invirase (saquinavir mesylate); lobenguane I 123 Injection for Intravenous Use (AdreView); lopromide Injection (Ultravist); loversol Injection (Optiray Injection); Iplex (Mecasermin Rinfabate [rDNA origin] Injection); lprivask; Irinotecan Hydrochloride (Camptosar Injection); Iron Sucrose Injection (Venofer); Istodax (Romidepsin for Injection); Itraconazole Injection (Sporanox Injection); Jevtana (Cabazitaxel Injection); Jonexa; Kalbitor (Ecallantide Injection); KCL in D5NS (Potassium Chloride in 5% Dextrose and Sodium Chloride Injection); KCL in D5W; KCL in NS; Kenalog 10 Injection (Triamcinolone Acetonide Injectable Suspension); Kepivance (Palifermin); Keppra Injection (Levetiracetam); Keratinocyte; KFG; Kinase Inhibitor; Kineret (Anakinra); Kinlytic (Urokinase Injection); Kinrix; Klonopin (clonazepam); Kytril Injection (Granisetron Hydrochloride); lacosamide Tablet and Injection (Vimpat); Lactated Ringers; Lanoxin Injection (Digoxin Injection); Lansoprazole for Injection (Prevacid I.V.); Lantus; Leucovorin Calcium (Leucovorin Calcium Injection); Lente (L); Leptin; Levemir; Leukine Sargramostim; Leuprolide Acetate; Levothyroxine; Levetiracetam (Keppra Injection); Lovenox; Levocamitine Injection (Carnitor Injection); Lexiscan (Regadenoson Injection); Lioresal Intrathecal (Baclofen Injection); Liraglutide [rDNA] Injection (Victoza); Lovenox (Enoxaparin Sodium Injection); Lucentis (Ranibizumab Injection); Lumizyme; Lupron (Leuprolide Acetate Injection); Lusedra (Fospropofol Disodium Injection); Maci; Magnesium Sulfate (Magnesium Sulfate Injection); Mannitol Injection (Mannitol IV); Marcaine (Bupivacaine Hydrochloride and Epinephrine Injection); Maxipime (Cefepime Hydrochloride for Injection); MDP Multidose Kit of Technetium Injection (Technetium Tc99m Medronate Injection); Mecasermin [rDNA origin] Injection (Increlex); Mecasermin Rinfabate [rDNA origin] Injection (Iplex); Melphalan Hcl Injection (Alkeran Injection); Methotrexate; Menactra; Menopur
(Menotropins Injection); Menotropins for Injection (Repronex); Methohexital Sodium for Injection (Brevital Sodium); Methyldopate Hydrochloride Injection, Solution (Methyldopate Hcl); Methylene Blue (Methylene Blue Injection); Methylprednisolone Acetate Injectable Suspension (Depo Medrol); MetMab; Metoclopramide Injection (Reglan Injection); Metrodin (Urofollitropin for Injection); Metronidazole Injection (Flagyl Injection); Miacalcin; Midazolam (Midazolam Injection); Mimpara (Cinacalet); Minocin Injection (Minocycline Inj); Minocycline Inj (Minocin Injection); Mipomersen; Mitoxantrone for Injection Concentrate (Novantrone); Morphine Injection (Duramorph); Morphine Sulfate XR Liposome Injection (DepoDur); Morrhuate Sodium (Morrhuate Sodium Injection); Motesanib; Mozobil (Plerixafor Injection); Multihance (Gadobenate Dimeglumine Injection); Multiple Electrolytes and Dextrose Injection; Multiple Electrolytes Injection; Mylotarg (Gemtuzumab Ozogamicin for Injection); Myozyme (Alglucosidase alfa); Nafcillin Injection (Nafcillin Sodium); Nafcillin Sodium (Nafcillin Injection); Naltrexone XR Inj (Vivitrol); Naprosyn (naproxen); NeoProfen (lbuprofen Lysine Injection); Nandrol Decanoate; Neostigmine Methylsulfate (Neostigmine Methylsulfate Injection); NEO-GAA; NeoTect (Technetium Tc 99m Depreotide Injection); Nephramine (Essential Amino Acid Injection); Neulasta (pegfilgrastim); Neupogen (Filgrastim); Novolin; Novolog; NeoRecormon; Neutrexin (Trimetrexate Glucuronate Inj); NPH (N); Nexterone (Amiodarone HCl Injection); Norditropin (Somatropin Injection); Normal Saline (Sodium Chloride Injection); Novantrone (Mitoxantrone for Injection Concentrate); Novolin 70/30 Innolet (70% NPH, Human Insulin Isophane Suspension and 30% Regular, Human Insulin Injection); NovoLog (Insulin Aspart [rDNA origin] Inj); Nplate (romiplostim); Nutropin (Somatropin (rDNA origin) for Inj); Nutropin AQ; Nutropin Depot (Somatropin (rDNA origin) for Inj); Octreotide Acetate Injection (Sandostatin LAR); Ocrelizumab; Ofatumumab Injection (Arzerra); Olanzapine Extended Release Injectable Suspension (Zyprexa Relprevv); Omnitarg; Omnitrope (Somatropin [rDNA origin] Injection); Ondansetron Hydrochloride Injection (Zofran Injection); OptiMARK (Gadoversetamide Injection); Optiray Injection (loversol Injection); Orencia; Osmitrol Injection in Aviva (Mannitol Injection in Aviva Plastic Vessel); Osmitrol Injection in Viaflex (Mannitol Injection in Viaflex Plastic Vessel); Osteoprotegrin; Ovidrel (Choriogonadotropin Alfa Injection); Oxacillin (Oxacillin for Injection); Oxaliplatin Injection (Eloxatin); Oxytocin Injection (Pitocin); Paliperidone Palmitate Extended-Release Injectable Suspension (Invega Sustenna); Pamidronate Disodium Injection (Pamidronate Disodium Injection); Panitumumab Injection for Intravenous Use (Vectibix); Papaverine Hydrochloride Injection (Papaverine Injection); Papaverine Injection (Papaverine Hydrochloride Injection); Parathyroid Hormone; Paricalcitol Injection Fliptop Vial (Zemplar Injection); PARP Inhibitor; Pediarix; PEGlntron; Peginterferon; Pegfilgrastim; Penicillin G Benzathine and Penicillin G Procaine; Pentetate Calcium Trisodium Inj (Ca-DTPA); Pentetate Zinc Trisodium Injection (Zn-DTPA); Pepcid Injection (Famotidine Injection); Pergonal; Pertuzumab; Phentolamine Mesylate (Phentolamine Mesylate for Injection); Physostigmine Salicylate (Physostigmine Salicylate (injection)); Physostigmine Salicylate (injection) (Physostigmine Salicylate); Piperacillin and Tazobactam Injection (Zosyn); Pitocin (Oxytocin Injection); Plasma-Lyte 148 (Multiple Electrolytes Inj); Plasma-Lyte 56 and Dextrose (Multiple Electrolytes and Dextrose Injection in Viaflex Plastic Vessel); PlasmaLyte; Plerixafor Injection (Mozobil); Polidocanol Injection (Asclera); Potassium Chloride; Pralatrexate Solution for Intravenous Injection (Folotyn); Pramlintide Acetate Injection (Symlin); Premarin Injection (Conjugated Estrogens for Injection); Prep kit for Technetium Tc99 Sestamibi for Injection (Cardiolite); Prevacid I.V. (Lansoprazole for Injection); Primaxin I.V. (Imipenem and Cilastatin for Injection); Prochymal; Procrit; Progesterone; ProHance (Gadoteridol Injection Solution); Prolia (Denosumab Injection); Promethazine HCl Injection (Promethazine Hydrochloride Injection); Propranolol Hydrochloride Injection (Propranolol Hydrochloride Injection); Quinidine Gluconate Injection (Quinidine Injection); Quinidine Injection (Quinidine Gluconate Injection); R-Gene 10 (Arginine Hydrochloride Injection); Ranibizumab Injection (Lucentis); Ranitidine Hydrochloride Injection (Zantac Injection); Raptiva; Reclast (Zoledronic Acid Injection); Recombivarix HB; Regadenoson Injection (Lexiscan); Reglan Injection (Metoclopramide Injection); Remicade; Renagel; Renvela (Sevelamer Carbonate); Repronex (Menotropins for Injection); Retrovir IV (Zidovudine Injection); rhApo2L/TRAIL; Ringers and 5% Dextrose Injection (Ringers in Dextrose); Ringers Injection (Ringers Injection);
Rituxan; Rituximab; Rocephin (ceftriaxone); Rocuronium Bromide Injection (Zemuron); Roferon-A (interferon alfa-2a); Romazicon (flumazenil); Romidepsin for Injection (Istodax); Saizen (Somatropin Injection); Sandostatin LAR (Octreotide Acetate Injection); Scerostin Ab; Sensipar (cinacalcet); Sensorcaine (Bupivacaine HCl Injections); Septocaine (Articane HCl and Epinephrine Injection); Serostim LQ (Somatropin (rDNA origin) Injection); Simponi Injection (Golimumab Injection); Sodium Acetate (Sodium Acetate Injection); Sodium Bicarbonate (Sodium Bicarbonate 5% Injection); Sodium Lactate (Sodium Lactate Injection in AVIVA); Sodium Phenylacetate and Sodium Benzoate Injection (Ammonul); Somatropin (rDNA origin) for Inj (Nutropin); Sporanox Injection (Itraconazole Injection); Stelara Injection (Ustekinumab); Stemgen; Sufenta (Sufentanil Citrate Injection); Sufentanil Citrate Injection (Sufenta); Sumavel; Sumatriptan Injection (Alsuma); Symlin; Symlin Pen; Systemic Hedgehog Antagonist; Synvisc-One (Hylan G-F 20 Single Intra-articular Injection); Tarceva; Taxotere (Docetaxel for Injection); Technetium Tc 99m; Telavancin for Injection (Vibativ); Temsirolimus Injection (Torisel); Tenormin I.V. Injection (Atenolol Inj); Teriparatide (rDNA origin) Injection (Forteo); Testosterone Cypionate; Testosterone Enanthate; Testosterone Propionate; Tev-Tropin (Somatropin, rDNA Origin, for Injection); tgAAC94; Thallous Chloride; Theophylline; Thiotepa (Thiotepa Injection); Thymoglobulin (Anti-Thymocyte Globulin (Rabbit); Thyrogen (Thyrotropin Alfa for Injection); Ticarcillin Disodium and Clavulanate Potassium Galaxy (Timentin Injection); Tigan Injection (Trimethobenzamide Hydrochloride Injectable); Timentin Injection (Ticarcillin Disodium and Clavulanate Potassium Galaxy); TNKase; Tobramycin Injection (Tobramycin Injection); Tocilizumab Injection (Actemra); Torisel (Temsirolimus Injection); Totect (Dexrazoxane for Injection, Intravenous Infusion Only); Trastuzumab-DM1; Travasol (Amino Acids (Injection)); Treanda (Bendamustine Hydrochloride Injection); Trelstar (Triptorelin Pamoate for Injectable Suspension); Triamcinolone Acetonide; Triamcinolone Diacetate; Triamcinolone Hexacetonide Injectable Suspension (Aristospan Injection 20 mg); Triesence (Triamcinolone Acetonide Injectable Suspension); Trimethobenzamide Hydrochloride Injectable (Tigan Injection); Trimetrexate Glucuronate Inj (Neutrexin); Triptorelin Pamoate for Injectable Suspension
(Trelstar); Twinject; Trivaris (Triamcinolone Acetonide Injectable Suspension); Trisenox (Arsenic Trioxide Injection); Twinrix; Typhoid Vi; Ultravist (lopromide Injection); Urofollitropin for Injection (Metrodin); Urokinase Injection (Kinlytic); Ustekinumab (Stelara Injection); Ultralente (U); Valium (diazepam); Valproate Sodium Injection (Depacon); Valtropin (Somatropin Injection); Vancomycin Hydrochloride (Vancomycin Hydrochloride Injection); Vancomycin Hydrochloride Injection (Vancomycin Hydrochloride); Vaprisol (Conivaptan Hcl Injection); VAQTA; Vasovist (Gadofosveset Trisodium Injection for Intravenous Use); Vectibix (Panitumumab Injection for Intravenous Use); Venofer (Iron Sucrose Injection); Verteporfin Inj (Visudyne); Vibativ (Telavancin for Injection); Victoza (Liraglutide [rDNA] Injection); Vimpat (lacosamide Tablet and Injection); Vinblastine Sulfate (Vinblastine Sulfate Injection); Vincasar PFS (Vincristine Sulfate Injection); Victoza; Vincristine Sulfate (Vincristine Sulfate Injection); Visudyne (Verteporfin Inj); Vitamin B-12; Vivitrol (Naltrexone XR Inj); Voluven (Hydroxyethyl Starch in Sodium Chloride Injection); Xeloda; Xenical (orlistat); Xeomin (Incobotulinumtoxin A for Injection); Xolair; Zantac Injection (Ranitidine Hydrochloride Injection); Zemplar Injection (Paricalcitol Injection Fliptop Vial); Zemuron (Rocuronium Bromide Injection); Zenapax (daclizumab); Zevalin; Zidovudine Injection (Retrovir IV); Zithromax Injection (Azithromycin); Zn-DTPA (Pentetate Zinc Trisodium Injection); Zofran Injection (Ondansetron Hydrochloride Injection); Zingo; Zoledronic Acid for Inj (Zometa); Zoledronic Acid Injection (Reclast); Zometa (Zoledronic Acid for Inj); Zosyn (Piperacillin and Tazobactam Injection); Zyprexa Relprevv (Olanzapine Extended Release Injectable Suspension)
LIQUID DRUGS (NON-INJECTABLE)
Abilify; AccuNeb (Albuterol Sulfate Inhalation Solution); Actidose Aqua (Activated Charcoal Suspension); Activated Charcoal Suspension (Actidose Aqua); Advair; Agenerase Oral Solution (Amprenavir Oral Solution); Akten (Lidocaine Hydrochloride Ophthalmic Gel); Alamast (Pemirolast Potassium Ophthalmic Solution); Albumin (Human) 5% Solution (Buminate 5%); Albuterol Sulfate Inhalation Solution; Alinia; Alocril; Alphagan; Alrex; Alvesco; Amprenavir Oral Solution; Analpram-HC; Arformoterol Tartrate Inhalation Solution (Brovana); Aristospan Injection 20 mg (Triamcinolone Hexacetonide Injectable Suspension); Asacol; Asmanex; Astepro; Astepro (Azelastine Hydrochloride Nasal Spray); Atrovent Nasal Spray (Ipratropium Bromide Nasal Spray); Atrovent Nasal Spray 0.06; Augmentin ES-600; Azasite (Azithromycin Ophthalmic Solution); Azelaic Acid (Finacea Gel); Azelastine Hydrochloride Nasal Spray (Astepro); Azelex (Azelaic Acid Cream); Azopt (Brinzolamide Ophthalmic Suspension); Bacteriostatic Saline; Balanced Salt; Bepotastine; Bactroban Nasal; Bactroban; Beclovent; Benzac W; Betimol; Betoptic S; Bepreve; Bimatoprost Ophthalmic Solution; Bleph 10 (Sulfacetamide Sodium Ophthalmic Solution 10%); Brinzolamide Ophthalmic Suspension (Azopt); Bromfenac Ophthalmic Solution (Xibrom); Bromhist; Brovana (Arformoterol Tartrate Inhalation Solution); Budesonide Inhalation Suspension (Pulmicort Respules); Cambia (Diclofenac Potassium for Oral Solution); Capex; Carac; Carboxine-PSE; Camitor; Cayston (Aztreonam for Inhalation Solution); Cellcept; Centany; Cerumenex; Ciloxan Ophthalmic Solution (Ciprofloxacin HCL Ophthalmic Solution); Ciprodex; Ciprofloxacin HCL Ophthalmic Solution (Ciloxan Ophthalmic Solution); Clemastine Fumarate Syrup (Clemastine Fumarate Syrup); CoLyte (PEG Electrolytes Solution); Combiven; Comtan; Condylox; Cordran; Cortisporin Ophthalmic Suspension; Cortisporin Otic Suspension; Cromolyn Sodium Inhalation Solution (Intal Nebulizer Solution); Cromolyn Sodium Ophthalmic Solution (Opticrom); Crystalline Amino Acid Solution with Electrolytes (Aminosyn Electrolytes); Cutivate; Cuvposa (Glycopyrrolate Oral Solution); Cyanocobalamin (CaloMist Nasal Spray); Cyclosporine Oral Solution (Gengraf Oral Solution); Cyclogyl; Cysview (Hexaminolevulinate Hydrochloride Intravesical Solution); DermOtic Oil (Fluocinolone Acetonide Oil Ear Drops); Desmopressin Acetate Nasal Spray; DDAVP; Derma-Smoothe/FS; Dexamethasone Intensol; Dianeal Low Calcium; Dianeal PD; Diclofenac Potassium for Oral Solution (Cambia); Didanosine Pediatric Powder for Oral Solution (Videx); Differin; Dilantin 125 (Phenytoin Oral Suspension); Ditropan; Dorzolamide Hydrochloride Ophthalmic Solution (Trusopt); Dorzolamide Hydrochloride-Timolol Maleate Ophthalmic Solution (Cosopt); Dovonex Scalp (Calcipotriene Solution); Doxycycline Calcium Oral Suspension (Vibramycin Oral); Efudex; Elaprase (Idursulfase Solution); Elestat (Epinastine HCl Ophthalmic Solution); Elocon; Epinastine HCl Ophthalmic Solution
(Elestat); Epivir HBV; Epogen (Epoetin alfa); Erythromycin Topical Solution 1.5% (Staticin); Ethiodol (Ethiodized Oil); Ethosuximide Oral Solution (Zarontin Oral Solution); Eurax; Extraneal (Icodextrin Peritoneal Dialysis Solution); Felbatol; Feridex I.V. (Ferumoxides Injectable Solution); Flovent; Floxin Otic (Ofloxacin Otic Solution); Flo-Pred (Prednisolone Acetate Oral Suspension); Fluoroplex; Flunisolide Nasal Solution (Flunisolide Nasal Spray 0.025%); Fluorometholone Ophthalmic Suspension (FML); Flurbiprofen Sodium Ophthalmic Solution (Ocufen); FML; Foradil; Formoterol Fumarate Inhalation Solution (Perforomist); Fosamax; Furadantin (Nitrofurantoin Oral Suspension); Furoxone; Gammagard Liquid (Immune Globulin Intravenous (Human) 10%); Gantrisin (Acetyl Sulfisoxazole Pediatric Suspension); Gatifloxacin Ophthalmic Solution (Zymar); Gengraf Oral Solution (Cyclosporine Oral Solution); Glycopyrrolate Oral Solution (Cuvposa); Halcinonide Topical Solution (Halog Solution); Halog Solution (Halcinonide Topical Solution); HEP-LOCK U/P (Preservative-Free Heparin Lock Flush Solution); Heparin Lock Flush Solution (Hepflush 10); Hexaminolevulinate Hydrochloride Intravesical Solution (Cysview); Hydrocodone Bitartrate and Acetaminophen Oral Solution (Lortab Elixir); Hydroquinone 3% Topical Solution (Melquin-3 Topical Solution); IAP Antagonist; Isopto; Ipratropium Bromide Nasal Spray (Atrovent Nasal Spray); Itraconazole Oral Solution (Sporanox Oral Solution); Ketorolac Tromethamine Ophthalmic Solution (Acular LS); Kaletra; Lanoxin; Lexiva; Leuprolide Acetate for Depot Suspension (Lupron Depot 11.25 mg); Levobetaxolol Hydrochloride Ophthalmic Suspension (Betaxon); Levocarnitine Tablets, Oral Solution, Sugar-Free (Carnitor); Levofloxacin Ophthalmic Solution 0.5% (Quixin); Lidocaine HCl Sterile Solution (Xylocaine MPF Sterile Solution); Lok Pak (Heparin Lock Flush Solution); Lorazepam Intensol; Lortab Elixir (Hydrocodone Bitartrate and Acetaminophen Oral Solution); Lotemax (Loteprednol Etabonate Ophthalmic Suspension); Loteprednol Etabonate Ophthalmic Suspension (Alrex); Low Calcium Peritoneal Dialysis Solutions (Dianeal Low Calcium); Lumigan (Bimatoprost Ophthalmic Solution 0.03% for Glaucoma); Lupron Depot 11.25 mg (Leuprolide Acetate for Depot Suspension); Megestrol Acetate Oral Suspension (Megestrol Acetate Oral Suspension); MEK Inhibitor; Mepron; Mesnex; Mestinon; Mesalamine Rectal Suspension Enema
(Rowasa); Melquin-3 Topical Solution (Hydroquinone 3% Topical Solution); MetMab; Methyldopate Hcl (Methyldopate Hydrochloride Injection, Solution); Methylin Oral Solution (Methylphenidate HCl Oral Solution 5 mg/5 mL and 10 mg/5 mL); Methylprednisolone Acetate Injectable Suspension (Depo Medrol); Methylphenidate HCl Oral Solution 5 mg/5 mL and 10 mg/5 mL (Methylin Oral Solution); Methylprednisolone sodium succinate (Solu Medrol); Metipranolol Ophthalmic Solution (Optipranolol); Migranal; Miochol-E (Acetylcholine Chloride Intraocular Solution); Micro-K for Liquid Suspension (Potassium Chloride Extended Release Formulation for Liquid Suspension); Minocin (Minocycline Hydrochloride Oral Suspension); Nasacort; Neomycin and Polymyxin B Sulfates and Hydrocortisone; Nepafenac Ophthalmic Suspension (Nevanac); Nevanac (Nepafenac Ophthalmic Suspension); Nitrofurantoin Oral Suspension (Furadantin); Noxafil (Posaconazole Oral Suspension); Nystatin (oral) (Nystatin Oral Suspension); Nystatin Oral Suspension (Nystatin (oral)); Ocufen (Flurbiprofen Sodium Ophthalmic Solution); Ofloxacin Ophthalmic Solution (Ofloxacin Ophthalmic Solution); Ofloxacin Otic Solution (Floxin Otic); Olopatadine Hydrochloride Ophthalmic Solution (Pataday); Opticrom (Cromolyn Sodium Ophthalmic Solution); Optipranolol (Metipranolol Ophthalmic Solution); Patanol; Pediapred; PerioGard; Phenytoin Oral Suspension (Dilantin 125); Phisohex; Posaconazole Oral Suspension (Noxafil); Potassium Chloride Extended Release Formulation for Liquid Suspension (Micro-K for Liquid Suspension); Pataday (Olopatadine Hydrochloride Ophthalmic Solution); Patanase Nasal Spray (Olopatadine Hydrochloride Nasal Spray); PEG Electrolytes Solution (CoLyte); Pemirolast Potassium Ophthalmic Solution (Alamast); Penlac (Ciclopirox Topical Solution); PENNSAID (Diclofenac Sodium Topical Solution); Perforomist (Formoterol Fumarate Inhalation Solution); Peritoneal Dialysis Solution; Phenylephrine Hydrochloride Ophthalmic Solution (Neo-Synephrine); Phospholine Iodide (Echothiophate Iodide for Ophthalmic Solution); Podofilox (Podofilox Topical Solution); Pred Forte (Prednisolone Acetate Ophthalmic Suspension); Pralatrexate Solution for Intravenous Injection (Folotyn); Pred Mild; Prednisone Intensol; Prednisolone Acetate Ophthalmic Suspension (Pred Forte); Prevacid; PrismaSol Solution (Sterile Hemofiltration Hemodiafiltration Solution); ProAir; Proglycem;
ProHance (Gadoteridol Injection Solution); Proparacaine Hydrochloride Ophthalmic Solution (Alcaine); Propine; Pulmicort; Pulmozyme; Quixin (Levofloxacin Ophthalmic Solution 0.5%); QVAR; Rapamune; Rebetol; Relacon-HC; Rotarix (Rotavirus Vaccine, Live, Oral Suspension); Rotavirus Vaccine, Live, Oral Suspension (Rotarix); Rowasa (Mesalamine Rectal Suspension Enema); Sabril (Vigabatrin Oral Solution); Sacrosidase Oral Solution (Sucraid); Sandimmune; Sepra; Serevent Diskus; Solu Cortef (Hydrocortisone Sodium Succinate); Solu Medrol (Methylprednisolone sodium succinate); Spiriva; Sporanox Oral Solution (Itraconazole Oral Solution); Staticin (Erythromycin Topical Solution 1.5%); Stalevo; Starlix; Sterile Hemofiltration Hemodiafiltration Solution (PrismaSol Solution); Stimate; Sucralfate (Carafate Suspension); Sulfacetamide Sodium Ophthalmic Solution 10% (Bleph 10); Synarel Nasal Solution (Nafarelin Acetate Nasal Solution for Endometriosis); Taclonex Scalp (Calcipotriene and Betamethasone Dipropionate Topical Suspension); Tamiflu; Tobi; TobraDex; Tobradex ST (Tobramycin/Dexamethasone Ophthalmic Suspension 0.3%/0.05%); Tobramycin/Dexamethasone Ophthalmic Suspension 0.3%/0.05% (Tobradex ST); Timolol; Timoptic; Travatan Z; Treprostinil Inhalation Solution (Tyvaso); Trusopt (Dorzolamide Hydrochloride Ophthalmic Solution); Tyvaso (Treprostinil Inhalation Solution); Ventolin; Vfend; Vibramycin Oral (Doxycycline Calcium Oral Suspension); Videx (Didanosine Pediatric Powder for Oral Solution); Vigabatrin Oral Solution (Sabril); Viokase; Viracept; Viramune; Vitamin K1 (Fluid Colloidal Solution of Vitamin K1); Voltaren Ophthalmic (Diclofenac Sodium Ophthalmic Solution); Zarontin Oral Solution (Ethosuximide Oral Solution); Ziagen; Zyvox; Zymar (Gatifloxacin Ophthalmic Solution); Zymaxid (Gatifloxacin Ophthalmic Solution)
DRUG CLASSES
5-alpha-reductase inhibitors; 5-aminosalicylates; 5HT3 receptor antagonists; adamantane antivirals; adrenal cortical steroids; adrenal corticosteroid inhibitors; adrenergic bronchodilators; agents for hypertensive emergencies; agents for pulmonary hypertension; aldosterone receptor antagonists; alkylating agents; alpha-adrenoreceptor antagonists; alpha-glucosidase inhibitors; alternative medicines; amebicides; aminoglycosides; aminopenicillins; aminosalicylates; amylin analogs; Analgesic Combinations; Analgesics; androgens and anabolic steroids; angiotensin converting enzyme inhibitors; angiotensin II inhibitors; anorectal preparations; anorexiants; antacids; anthelmintics; anti-angiogenic ophthalmic agents; anti-CTLA-4 monoclonal antibodies; anti-infectives; antiadrenergic agents, centrally acting; antiadrenergic agents, peripherally acting; antiandrogens; antianginal agents; antiarrhythmic agents; antiasthmatic combinations; antibiotics/antineoplastics; anticholinergic antiemetics; anticholinergic antiparkinson agents; anticholinergic bronchodilators; anticholinergic chronotropic agents; anticholinergics/antispasmodics; anticoagulants; anticonvulsants; antidepressants; antidiabetic agents; antidiabetic combinations; antidiarrheals; antidiuretic hormones; antidotes; antiemetic/antivertigo agents; antifungals; antigonadotropic agents; antigout agents; antihistamines; antihyperlipidemic agents; antihyperlipidemic combinations; antihypertensive combinations; antihyperuricemic agents; antimalarial agents; antimalarial combinations; antimalarial quinolines; antimetabolites; antimigraine agents; antineoplastic detoxifying agents; antineoplastic interferons; antineoplastic monoclonal antibodies; antineoplastics; antiparkinson agents; antiplatelet agents; antipseudomonal penicillins; antipsoriatics; antipsychotics; antirheumatics; antiseptic and germicides; antithyroid agents; antitoxins and antivenins; antituberculosis agents; antituberculosis combinations; antitussives; antiviral agents; antiviral combinations; antiviral interferons; anxiolytics, sedatives, and hypnotics; aromatase inhibitors; atypical antipsychotics; azole antifungals; bacterial vaccines; barbiturate anticonvulsants; barbiturates; BCR-ABL tyrosine kinase inhibitors; benzodiazepine anticonvulsants; benzodiazepines; beta-adrenergic blocking agents; beta-lactamase inhibitors; bile acid sequestrants; biologicals; bisphosphonates; bone resorption inhibitors; bronchodilator combinations; bronchodilators; calcitonin; calcium channel blocking agents; carbamate anticonvulsants; carbapenems; carbonic anhydrase inhibitor anticonvulsants; carbonic anhydrase inhibitors; cardiac stressing agents; cardioselective beta blockers; cardiovascular agents; catecholamines; CD20 monoclonal antibodies; CD33 monoclonal antibodies; CD52 monoclonal antibodies; central nervous system agents; cephalosporins; cerumenolytics; chelating agents; chemokine receptor antagonist; chloride channel activators; cholesterol absorption
inhibitors; cholinergic agonists; cholinergic muscle stimulants; cholinesterase inhibitors; CNS stimulants; coagulation modifiers; colony stimulating factors; contraceptives; corticotropin; coumarins and indandiones; cox-2 inhibitors; decongestants; dermatological agents; diagnostic radiopharmaceuticals; dibenzazepine anticonvulsants; digestive enzymes; dipeptidyl peptidase 4 inhibitors; diuretics; dopaminergic antiparkinsonism agents; drugs used in alcohol dependence; echinocandins; EGFR inhibitors; estrogen receptor antagonists; estrogens; expectorants; factor Xa inhibitors; fatty acid derivative anticonvulsants; fibric acid derivatives; first generation cephalosporins; fourth generation cephalosporins; functional bowel disorder agents; gallstone solubilizing agents; gamma-aminobutyric acid analogs; gamma-aminobutyric acid reuptake inhibitors; gamma-aminobutyric acid transaminase inhibitors; gastrointestinal agents; general anesthetics; genitourinary tract agents; GI stimulants; glucocorticoids; glucose elevating agents; glycopeptide antibiotics; glycoprotein platelet inhibitors; glycylcyclines; gonadotropin releasing hormones; gonadotropin-releasing hormone antagonists; gonadotropins; group I antiarrhythmics; group II antiarrhythmics; group Ill antiarrhythmics; group IV antiarrhythmics; group V antiarrhythmics; growth hormone receptor blockers; growth hormones; H. pylori eradication agents; H2 antagonists; hematopoietic stem cell mobilizer; heparin antagonists; heparins; HER2 inhibitors; herbal products; histone deacetylase inhibitors; hormone replacement therapy; hormones; hormones/antineoplastics; hydantoin anticonvulsants; illicit (street) drugs; immune globulins; immunologic agents; immunosuppressive agents; impotence agents; in vivo diagnostic biologicals; incretin mimetics; inhaled anti-infectives; inhaled corticosteroids; inotropic agents; insulin; insulin-like growth factor; integrase strand transfer inhibitor; interferons; intravenous nutritional products; iodinated contrast media; ionic iodinated contrast media; iron products; ketolides; laxatives; leprostatics; leukotriene modifiers; lincomycin derivatives; lipoglycopeptides; local injectable anesthetics; loop diuretics; lung surfactants; lymphatic staining agents; lysosomal enzymes; macrolide derivatives; macrolides; magnetic resonance imaging contrast media; mast cell stabilizers; medical gas; meglitinides; metabolic agents; methylxanthines; mineralocorticoids; minerals and electrolytes;
miscellaneous agents; miscellaneous analgesics; miscellaneous antibiotics; miscellaneous anticonvulsants; miscellaneous antidepressants; miscellaneous antidiabetic agents; miscellaneous antiemetics; miscellaneous antifungals; miscellaneous antihyperlipidemic agents; miscellaneous antimalarials; miscellaneous antineoplastics; miscellaneous antiparkinson agents; miscellaneous antipsychotic agents; miscellaneous antituberculosis agents; miscellaneous antivirals; miscellaneous anxiolytics, sedatives and hypnotics; miscellaneous biologicals; miscellaneous bone resorption inhibitors; miscellaneous cardiovascular agents; miscellaneous central nervous system agents; miscellaneous coagulation modifiers; miscellaneous diuretics; miscellaneous genitourinary tract agents; miscellaneous GI agents; miscellaneous hormones; miscellaneous metabolic agents; miscellaneous ophthalmic agents; miscellaneous otic agents; miscellaneous respiratory agents; miscellaneous sex hormones; miscellaneous topical agents; miscellaneous uncategorized agents; miscellaneous vaginal agents; mitotic inhibitors; monoamine oxidase inhibitors; monoclonal antibodies; mouth and throat products; mTOR inhibitors; mTOR kinase inhibitors; mucolytics; multikinase inhibitors; muscle relaxants; mydriatics; narcotic analgesic combinations; narcotic analgesics; nasal anti-infectives; nasal antihistamines and decongestants; nasal lubricants and irrigations; nasal preparations; nasal steroids; natural penicillins; neuraminidase inhibitors; neuromuscular blocking agents; next generation cephalosporins; nicotinic acid derivatives; nitrates; NNRTIs; non-cardioselective beta blockers; non-iodinated contrast media; non-ionic iodinated contrast media; non-sulfonylureas; nonsteroidal anti-inflammatory agents; norepinephrine reuptake inhibitors; norepinephrine-dopamine reuptake inhibitors; nucleoside reverse transcriptase inhibitors (NRTIs); nutraceutical products; nutritional products; ophthalmic anesthetics; ophthalmic anti-infectives; ophthalmic anti-inflammatory agents; ophthalmic antihistamines and decongestants; ophthalmic diagnostic agents; ophthalmic glaucoma agents; ophthalmic lubricants and irrigations; ophthalmic preparations; ophthalmic steroids; ophthalmic steroids with anti-infectives; ophthalmic surgical agents; oral nutritional supplements; otic anesthetics; otic anti-infectives; otic preparations; otic steroids; otic steroids with anti-infectives;
oxazolidinedione anticonvulsants; parathyroid hormone and analogs; penicillinase resistant penicillins; penicillins; peripheral opioid receptor antagonists; peripheral vasodilators; peripherally acting antiobesity agents; phenothiazine antiemetics; phenothiazine antipsychotics; phenylpiperazine antidepressants; plasma expanders; platelet aggregation inhibitors; platelet-stimulating agents; polyenes; potassium-sparing diuretics; probiotics; progesterone receptor modulators; progestins; prolactin inhibitors; prostaglandin D2 antagonists; protease inhibitors; proton pump inhibitors; psoralens; psychotherapeutic agents; psychotherapeutic combinations; purine nucleosides; pyrrolidine anticonvulsants; quinolones; radiocontrast agents; radiologic adjuncts; radiologic agents; radiologic conjugating agents; radiopharmaceuticals; RANK ligand inhibitors; recombinant human erythropoietins; renin inhibitors; respiratory agents; respiratory inhalant products; rifamycin derivatives; salicylates; sclerosing agents; second generation cephalosporins; selective estrogen receptor modulators; selective serotonin reuptake inhibitors; serotonin-norepinephrine reuptake inhibitors; serotoninergic neuroenteric modulators; sex hormone combinations; sex hormones; skeletal muscle relaxant combinations; skeletal muscle relaxants; smoking cessation agents; somatostatin and somatostatin analogs; spermicides; statins; sterile irrigating solutions; streptomyces derivatives; succinimide anticonvulsants; sulfonamides; sulfonylureas; synthetic ovulation stimulants; tetracyclic antidepressants; tetracyclines; therapeutic radiopharmaceuticals; thiazide diuretics; thiazolidinediones; thioxanthenes; third generation cephalosporins; thrombin inhibitors; thrombolytics; thyroid drugs; tocolytic agents; topical acne agents; topical agents; topical anesthetics; topical anti-infectives; topical antibiotics; topical antifungals; topical antihistamines; topical antipsoriatics; topical antivirals; topical astringents; topical debriding agents; topical depigmenting agents; topical emollients; topical keratolytics; topical steroids; topical steroids with anti-infectives; toxoids; triazine anticonvulsants; tricyclic antidepressants; trifunctional monoclonal antibodies; tumor necrosis factor (TNF) inhibitors; tyrosine kinase inhibitors; ultrasound contrast media; upper respiratory combinations; urea anticonvulsants; urinary anti-infectives; urinary antispasmodics; urinary pH modifiers; uterotonic agents; vaccine; vaccine combinations; vaginal anti-infectives; vaginal
preparations; vasodilators; vasopressin antagonists; vasopressors; VEGFNEGFR inhibitors; viral vaccines; viscosupplementation agents; vitamin and mineral combinations; vitamins
DIAGNOSTIC TESTS
17-Hydroxyprogesterone; ACE (Angiotensin I converting enzyme); Acetaminophen; Acid phosphatase; ACTH; Activated clotting time; Activated protein C resistance; Adrenocorticotropic hormone (ACTH); Alanine aminotransferase (ALT); Albumin; Aldolase; Aldosterone; Alkaline phosphatase; Alkaline phosphatase (ALP); Alpha1-antitrypsin; Alpha-fetoprotein; Alpha-fetoprotien; Ammonia levels; Amylase; ANA (antinuclear antibodies); ANA (antinuclear antibodies); Angiotensin-converting enzyme (ACE); Anion gap; Anticardiolipin antibody; Anticardiolipin antibodies (ACA); Anti-centromere antibody; Antidiuretic hormone; Anti-DNA; Anti-Dnase-B; Anti-Gliadin antibody; Anti-glomerular basement membrane antibody; Anti-HBc (Hepatitis B core antibodies; Anti-HBs (Hepatitis B surface antibody; Antiphospholipid antibody; Anti-RNA polymerase; Anti-Smith (Sm) antibodies; Anti-Smooth Muscle antibody; Antistreptolysin 0 (ASO); Antithrombin Ill; Anti-Xa activity; Anti-Xa assay; Apolipoproteins; Arsenic; Aspartate aminotransferase (AST); B12; Basophil; Beta-2-Microglobulin; Beta-hydroxybutyrate; B-HCG; Bilirubin; Bilirubin, direct; Bilirubin, indirect; Bilirubin, total; Bleeding time; Blood gases (arterial); Blood urea nitrogen (BUN); BUN; BUN (blood urea nitrogen); CA 125; CA 15-3; CA 19-9; Calcitonin; Calcium; Calcium (ionized); Carbon monoxide (CO); Carcinoembryonic antigen (CEA); CBC; CEA; CEA (carcinoembryonic antigen); Ceruloplasmin; CH50Chloride; Cholesterol; Cholesterol, HDL; Clot lysis time; Clot retraction time; CMP; CO2; Cold agglutinins; Complement C3; Copper; Corticotrophin releasing hormone (CRH) stimulation test; Cortisol; Cortrosyn stimulation test; C-peptide; CPK (Total); CPK-MB; C-reactive protein; Creatinine; Creatinine kinase (CK); Cryoglobulins; DAT (Direct antiglobulin test); D-Dimer; Dexamethasone suppression test; DHEA-S; Dilute Russell viper venom; Elliptocytes; Eosinophil; Erythrocyte sedimentation rate (ESR); Estradiol; Estriol; Ethanol; Ethylene glycol; Euglobulin lysis; Factor V Leiden; Factor VIII inhibitor; Factor VIII level; Ferritin; Fibrin split products; Fibrinogen; Folate; Folate (serum; Fractional excretion of sodium (FENA); FSH (follicle stimulating factor); FTA-ABS; Gamma glutamyl transferase (GGT); Gastrin; GGTP (Gamma glutamyl transferase); Glucose; Growth hormone; Haptoglobin; HBeAg (Hepatitis Be antigen); HBs-Ag (Hepatitis B surface antigen); Helicobacter pylori; Hematocrit; Hematocrit (HCT); Hemoglobin; Hemoglobin AlC; Hemoglobin electrophoresis; Hepatitis A antibodies; Hepatitis C antibodies; IAT (Indirect antiglobulin test); Immunofixation (IFE); Iron; Lactate dehydrogenase (LDH); Lactic acid (lactate); LDH; LH (Leutinizing hormone; Lipase; Lupus anticoagulant; Lymphocyte; Magnesium; MCH (mean corpuscular hemoglobin; MCHC (mean corpuscular hemoglobin concentration); MCV (mean corpuscular volume); Methylmalonate; Monocyte; MPV (mean platelet volume); Myoglobin; Neutrophil; Parathyroid hormone (PTH); Phosphorus; Platelets (pit); Potassium; Prealbumin; Prolactin; Prostate specific antigen (PSA); Protein C; Protein S; PSA (prostate specific antigen); PT (Prothrombin time); PTT (Partial thromboplastin time); RDW (red cell distribution width); Renin; Rennin; Reticulocyte count; reticulocytes; Rheumatoid factor (RF); Sed Rate; Serum glutamic-pyruvic transaminase (SGPT; Serum protein electrophoresis (SPEP); Sodium; T3-resin uptake (T3RU); T4, Free; Thrombin time; Thyroid stimulating hormone (TSH); Thyroxine (T4); Total iron binding capacity (TIBC); Total protein; Transferrin; Transferrin saturation; Triglyceride (TG); Troponin; Uric acid; Vitamin B12; White blood cells (WBC); Widal test.

52. A drug primary packaging comprising the vessel and aqueous drug product or drug-containing solution according to any one of the preceding claims.
